SEQUENCE LISTING (898 sequences)

SEQ ID NO: 1 - RNA, 102 nt, Artificial Sequence
  Spy Cas9 sgRNA sequence
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 2 - RNA, 82 nt, Artificial Sequence
  Spy Cas9 sgRNA sequence
guuuuagagcuagaaauagcaaguuaaaauaaggcuaguccguuaucaacuugaaaaaguggcaccgagucggugcuuuuuu

SEQ ID NO: 3 - DNA, 3974 bp, Streptococcus pyogenes
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaa

SEQ ID NO: 4 - protein, 1423 aa, Streptococcus pyogenes
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 5 - RNA, 105 nt, Staphylococcus aureus
  misc_feature (2)..(22): n is a, c, g, or u
gnnnnnnnnnnnnnnnnnnnnnguuuuaguacucuggaaacagaaucuacuaaaacaaggcaaaugccguguuuaucucgucaacuuguuggcgaagauuuuuuu

SEQ ID NO: 6 - RNA, 83 nt, Staphylococcus aureus
guuuuaguacucuggaaacagaaucuacuaaaacaaggcaaaugccguguuuaucucgucaacuuguuggcgaagauuuuuuu

SEQ ID NO: 7 - DNA, 3345 bp, Staphylococcus aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaa

SEQ ID NO: 8 - protein, 1114 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYAYPYDVPDYAYPYDVPDYA

SEQ ID NO: 9 - DNA, 20 bp, Artificial Sequence
  20nt guide using novel PAM
aatgatagattagcttccta

SEQ ID NO: 10 - DNA, 20 bp, Artificial Sequence
  20nt guide using novel PAM
taggaagctaatctatcatt

SEQ ID NO: 11 - DNA, 25 bp, Artificial Sequence
  20nt guide using novel PAM
caccgtaggaagctaatctatcatt

SEQ ID NO: 12 - DNA, 25 bp, Artificial Sequence
  20nt guide using novel PAM
caaaaatgatagattagcttcctac

SEQ ID NO: 13 - DNA, 22 bp, Artificial Sequence
  22nt guide using novel PAM
taatgatagattagcttcctac

SEQ ID NO: 14 - DNA, 22 bp, Artificial Sequence
  22nt guide using novel PAM
gtaggaagctaatctatcatta

SEQ ID NO: 15 - DNA, 27 bp, Artificial Sequence
  22nt guide using novel PAM
caccggtaggaagctaatctatcatta

SEQ ID NO: 16 - DNA, 27 bp, Artificial Sequence
  22nt guide using novel PAM
caaataatgatagattagcttcctacc

SEQ ID NO: 17 - DNA, 21 bp, Artificial Sequence
  21nt guide using novel PAM
taatgatagattagcttccta

SEQ ID NO: 18 - DNA, 21 bp, Artificial Sequence
  21nt guide using novel PAM
taggaagctaatctatcatta

SEQ ID NO: 19 - DNA, 26 bp, Artificial Sequence
  21nt guide using novel PAM
caccgtaggaagctaatctatcatta

SEQ ID NO: 20 - DNA, 26 bp, Artificial Sequence
  21nt guide using novel PAM
caaataatgatagattagcttcctac

SEQ ID NO: 21 - DNA, 20 bp, Artificial Sequence
  20nt guide using novel PAM
taatgatagattagcttcct

SEQ ID NO: 22 - DNA, 20 bp, Artificial Sequence
  20nt guide using novel PAM
aggaagctaatctatcatta

SEQ ID NO: 23 - DNA, 25 bp, Artificial Sequence
  20nt guide using novel PAM
caccgaggaagctaatctatcatta

SEQ ID NO: 24 - DNA, 25 bp, Artificial Sequence
  20nt guide using novel PAM
caaataatgatagattagcttcctc

SEQ ID NO: 25 - DNA, 19 bp, Artificial Sequence
  19nt guide using novel PAM
aatgatagattagcttcct

SEQ ID NO: 26 - DNA, 19 bp, Artificial Sequence
  19nt guide using novel PAM
aggaagctaatctatcatt

SEQ ID NO: 27 - DNA, 24 bp, Artificial Sequence
  19nt guide using novel PAM
caccgaggaagctaatctatcatt

SEQ ID NO: 28 - DNA, 24 bp, Artificial Sequence
  19nt guide using novel PAM
caaaaatgatagattagcttcctc

SEQ ID NO: 29 - DNA, 18 bp, Artificial Sequence
  18nt guide using novel PAM
atgatagattagcttcct

SEQ ID NO: 30 - DNA, 18 bp, Artificial Sequence
  18nt guide using novel PAM
aggaagctaatctatcat

SEQ ID NO: 31 - DNA, 23 bp, Artificial Sequence
  18nt guide using novel PAM
caccgaggaagctaatctatcat

SEQ ID NO: 32 - DNA, 23 bp, Artificial Sequence
  18nt guide using novel PAM
caaaatgatagattagcttcctc

SEQ ID NO: 33 - DNA, 17 bp, Artificial Sequence
  17nt guide using novel PAM
tgatagattagcttcct

SEQ ID NO: 34 - DNA, 17 bp, Artificial Sequence
  17nt guide using novel PAM
aggaagctaatctatca

SEQ ID NO: 35 - DNA, 22 bp, Artificial Sequence
  17nt guide using novel PAM
caccgaggaagctaatctatca

SEQ ID NO: 36 - DNA, 22 bp, Artificial Sequence
  17nt guide using novel PAM
caaatgatagattagcttcctc

SEQ ID NO: 37 - DNA, 16 bp, Artificial Sequence
  16nt guide using novel PAM
gatagattagcttcct

SEQ ID NO: 38 - DNA, 16 bp, Artificial Sequence
  16nt guide using novel PAM
aggaagctaatctatc

SEQ ID NO: 39 - DNA, 21 bp, Artificial Sequence
  16nt guide using novel PAM
caccgaggaagctaatctatc

SEQ ID NO: 40 - DNA, 21 bp, Artificial Sequence
  16nt guide using novel PAM
caaagatagattagcttcctc

SEQ ID NO: 41 - DNA, 22 bp, Artificial Sequence
  22nt guide, mutation at position 4 in seed region
actcagctgtacacggactgca

SEQ ID NO: 42 - DNA, 22 bp, Artificial Sequence
  22nt guide, mutation at position 4 in seed region
actcagctgtacacggactgca

SEQ ID NO: 43 - DNA, 27 bp, Artificial Sequence
  22nt guide, mutation at position 4 in seed region
caccgactcagctgtacacggactgca

SEQ ID NO: 44 - DNA, 27 bp, Artificial Sequence
  22nt guide, mutation at position 4 in seed region
caaatgcagtccgtgtacagctgagtc

SEQ ID NO: 45 - DNA, 21 bp, Artificial Sequence
  21nt guide, mutation at position 4 in seed region
ctcagctgtacacggactgca

SEQ ID NO: 46 - DNA, 21 bp, Artificial Sequence
  21nt guide, mutation at position 4 in seed region
ctcagctgtacacggactgca

SEQ ID NO: 47 - DNA, 26 bp, Artificial Sequence
  21nt guide, mutation at position 4 in seed region
caccgctcagctgtacacggactgca

SEQ ID NO: 48 - DNA, 26 bp, Artificial Sequence
  21nt guide, mutation at position 4 in seed region
caaatgcagtccgtgtacagctgagc

SEQ ID NO: 49 - DNA, 20 bp, Artificial Sequence
  20nt guide, mutation at position 4 in seed region22nt guide, mutation at position 4 in seed region
tcagctgtacacggactgca

SEQ ID NO: 50 - DNA, 20 bp, Artificial Sequence
  20nt guide, mutation at position 4 in seed region22nt guide, mutation at position 4 in seed region
tcagctgtacacggactgca

SEQ ID NO: 51 - DNA, 25 bp, Artificial Sequence
  20nt guide, mutation at position 4 in seed region22nt guide, mutation at position 4 in seed region
caccgtcagctgtacacggactgca

SEQ ID NO: 52 - DNA, 25 bp, Artificial Sequence
  20nt guide, mutation at position 4 in seed region22nt guide, mutation at position 4 in seed region
caaatgcagtccgtgtacagctgac

SEQ ID NO: 53 - DNA, 19 bp, Artificial Sequence
  19nt guide, mutation at position 4 in seed region
cagctgtacacggactgca

SEQ ID NO: 54 - DNA, 19 bp, Artificial Sequence
  19nt guide, mutation at position 4 in seed region
cagctgtacacggactgca

SEQ ID NO: 55 - DNA, 24 bp, Artificial Sequence
  19nt guide, mutation at position 4 in seed region
caccgcagctgtacacggactgca

SEQ ID NO: 56 - DNA, 24 bp, Artificial Sequence
  19nt guide, mutation at position 4 in seed region
caaatgcagtccgtgtacagctgc

SEQ ID NO: 57 - DNA, 18 bp, Artificial Sequence
  18nt guide, mutation at position 4 in seed region
agctgtacacggactgca

SEQ ID NO: 58 - DNA, 18 bp, Artificial Sequence
  18nt guide, mutation at position 4 in seed region
agctgtacacggactgca

SEQ ID NO: 59 - DNA, 23 bp, Artificial Sequence
  18nt guide, mutation at position 4 in seed region
caccgagctgtacacggactgca

SEQ ID NO: 60 - DNA, 23 bp, Artificial Sequence
  18nt guide, mutation at position 4 in seed region
caaatgcagtccgtgtacagctc

SEQ ID NO: 61 - DNA, 17 bp, Artificial Sequence
  17nt guide, mutation at position 4 in seed region
gctgtacacggactgca

SEQ ID NO: 62 - DNA, 17 bp, Artificial Sequence
  17nt guide, mutation at position 4 in seed region
gctgtacacggactgca

SEQ ID NO: 63 - DNA, 22 bp, Artificial Sequence
  17nt guide, mutation at position 4 in seed region
caccggctgtacacggactgca

SEQ ID NO: 64 - DNA, 22 bp, Artificial Sequence
  17nt guide, mutation at position 4 in seed region
caaatgcagtccgtgtacagcc

SEQ ID NO: 65 - DNA, 16 bp, Artificial Sequence
  16nt guide, mutation at position 4 in seed region
ctgtacacggactgca

SEQ ID NO: 66 - DNA, 16 bp, Artificial Sequence
  16nt guide, mutation at position 4 in seed region
ctgtacacggactgca

SEQ ID NO: 67 - DNA, 21 bp, Artificial Sequence
  16nt guide, mutation at position 4 in seed region
caccgctgtacacggactgca

SEQ ID NO: 68 - DNA, 21 bp, Artificial Sequence
  16nt guide, mutation at position 4 in seed region
caaatgcagtccgtgtacagc

SEQ ID NO: 69 - DNA, 24 bp, Artificial Sequence
  24nt guide, mutation at position 3 in seed region
ccactcagctgtacacggaccaca

SEQ ID NO: 70 - DNA, 24 bp, Artificial Sequence
  24nt guide, mutation at position 3 in seed region
ccactcagctgtacacggaccaca

SEQ ID NO: 71 - DNA, 29 bp, Artificial Sequence
  24nt guide, mutation at position 3 in seed region
caccgccactcagctgtacacggaccaca

SEQ ID NO: 72 - DNA, 29 bp, Artificial Sequence
  24nt guide, mutation at position 3 in seed region
caaatgtggtccgtgtacagctgagtggc

SEQ ID NO: 73 - DNA, 23 bp, Artificial Sequence
  23nt guide, mutation at position 3 in seed region
cactcagctgtacacggaccaca

SEQ ID NO: 74 - DNA, 23 bp, Artificial Sequence
  23nt guide, mutation at position 3 in seed region
cactcagctgtacacggaccaca

SEQ ID NO: 75 - DNA, 28 bp, Artificial Sequence
  23nt guide, mutation at position 3 in seed region
caccgcactcagctgtacacggaccaca

SEQ ID NO: 76 - DNA, 28 bp, Artificial Sequence
  23nt guide, mutation at position 3 in seed region
caaatgtggtccgtgtacagctgagtgc

SEQ ID NO: 77 - DNA, 22 bp, Artificial Sequence
  22nt guide, mutation at position 3 in seed region
actcagctgtacacggaccaca

SEQ ID NO: 78 - DNA, 22 bp, Artificial Sequence
  22nt guide, mutation at position 3 in seed region
actcagctgtacacggaccaca

SEQ ID NO: 79 - DNA, 27 bp, Artificial Sequence
  22nt guide, mutation at position 3 in seed region
caccgactcagctgtacacggaccaca

SEQ ID NO: 80 - DNA, 27 bp, Artificial Sequence
  22nt guide, mutation at position 3 in seed region
caaatgtggtccgtgtacagctgagtc

SEQ ID NO: 81 - DNA, 21 bp, Artificial Sequence
  21nt guide, mutation at position 3 in seed region
ctcagctgtacacggaccaca

SEQ ID NO: 82 - DNA, 21 bp, Artificial Sequence
  21nt guide, mutation at position 3 in seed region
ctcagctgtacacggaccaca

SEQ ID NO: 83 - DNA, 26 bp, Artificial Sequence
  21nt guide, mutation at position 3 in seed region
caccgctcagctgtacacggaccaca

SEQ ID NO: 84 - DNA, 26 bp, Artificial Sequence
  21nt guide, mutation at position 3 in seed region
caaatgtggtccgtgtacagctgagc

SEQ ID NO: 85 - DNA, 20 bp, Artificial Sequence
  20nt guide, mutation at position 3 in seed region
tcagctgtacacggaccaca

SEQ ID NO: 86 - DNA, 20 bp, Artificial Sequence
  20nt guide, mutation at position 3 in seed region
tcagctgtacacggaccaca

SEQ ID NO: 87 - DNA, 25 bp, Artificial Sequence
  20nt guide, mutation at position 3 in seed region
caccgtcagctgtacacggaccaca

SEQ ID NO: 88 - DNA, 25 bp, Artificial Sequence
  20nt guide, mutation at position 3 in seed region
caaatgtggtccgtgtacagctgac

SEQ ID NO: 89 - DNA, 19 bp, Artificial Sequence
  19nt guide, mutation at position 3 in seed region
cagctgtacacggaccaca

SEQ ID NO: 90 - DNA, 19 bp, Artificial Sequence
  19nt guide, mutation at position 3 in seed region
cagctgtacacggaccaca

SEQ ID NO: 91 - DNA, 24 bp, Artificial Sequence
  19nt guide, mutation at position 3 in seed region
caccgcagctgtacacggaccaca

SEQ ID NO: 92 - DNA, 24 bp, Artificial Sequence
  19nt guide, mutation at position 3 in seed region
caaatgtggtccgtgtacagctgc

SEQ ID NO: 93 - DNA, 18 bp, Artificial Sequence
  18nt guide, mutation at position 3 in seed region
agctgtacacggaccaca

SEQ ID NO: 94 - DNA, 18 bp, Artificial Sequence
  18nt guide, mutation at position 3 in seed region
agctgtacacggaccaca

SEQ ID NO: 95 - DNA, 23 bp, Artificial Sequence
  18nt guide, mutation at position 3 in seed region
caccgagctgtacacggaccaca

SEQ ID NO: 96 - DNA, 23 bp, Artificial sequence
  18nt guide, mutation at position 3 in seed region
caaatgtggtccgtgtacagctc

SEQ ID NO: 97 - DNA, 22 bp, Artificial Sequence
  22nt guide, mutation at position 3 of seed region
actcagctgtacacggacctca

SEQ ID NO: 98 - DNA, 22 bp, Artificial Sequence
  22nt guide, mutation at position 3 of seed region
actcagctgtacacggacctca

SEQ ID NO: 99 - DNA, 27 bp, Artificial Sequence
  22nt guide, mutation at position 3 of seed region
caccgactcagctgtacacggacctca

SEQ ID NO: 100 - DNA, 27 bp, Artificial Sequence
  22nt guide, mutation at position 3 of seed region
caaatgaggtccgtgtacagctgagtc

SEQ ID NO: 101 - DNA, 21 bp, Artificial Sequence
  21nt guide, mutation at position 3 of seed region
ctcagctgtacacggacctca

SEQ ID NO: 102 - DNA, 21 bp, Artificial Sequence
  21nt guide, mutation at position 3 of seed region
ctcagctgtacacggacctca

SEQ ID NO: 103 - DNA, 26 bp, Artificial Sequence
  21nt guide, mutation at position 3 of seed region
caccgctcagctgtacacggacctca

SEQ ID NO: 104 - DNA, 26 bp, Artificial Sequence
  21nt guide, mutation at position 3 of seed region
caaatgaggtccgtgtacagctgagc

SEQ ID NO: 105 - DNA, 20 bp, Artificial Sequence
  20nt guide, mutation at position 3 of seed region
tcagctgtacacggacctca

SEQ ID NO: 106 - DNA, 20 bp, Artificial Sequence
  20nt guide, mutation at position 3 of seed region
tcagctgtacacggacctca

SEQ ID NO: 107 - DNA, 25 bp, Artificial Sequence
  20nt guide, mutation at position 3 of seed region
caccgtcagctgtacacggacctca

SEQ ID NO: 108 - DNA, 25 bp, Artificial Sequence
  20nt guide, mutation at position 3 of seed region
caaatgaggtccgtgtacagctgac

SEQ ID NO: 109 - DNA, 19 bp, Artificial Sequence
  19nt guide, mutation at position 3 of seed region
cagctgtacacggacctca

SEQ ID NO: 110 - DNA, 19 bp, Artificial Sequence
  19nt guide, mutation at position 3 of seed region
cagctgtacacggacctca

SEQ ID NO: 111 - DNA, 24 bp, Artificial Sequence
  19nt guide, mutation at position 3 of seed region
caccgcagctgtacacggacctca

SEQ ID NO: 112 - DNA, 24 bp, Artificial Sequence
  19nt guide, mutation at position 3 of seed region
caaatgaggtccgtgtacagctgc

SEQ ID NO: 113 - DNA, 18 bp, Artificial Sequence
  18nt guide, mutation at position 3 of seed region
agctgtacacggacctca

SEQ ID NO: 114 - DNA, 18 bp, Artificial Sequence
  18nt guide, mutation at position 3 of seed region
agctgtacacggacctca

SEQ ID NO: 115 - DNA, 23 bp, Artificial Sequence
  18nt guide, mutation at position 3 of seed region
caccgagctgtacacggacctca

SEQ ID NO: 116 - DNA, 23 bp, Artificial Sequence
  18nt guide, mutation at position 3 of seed region
caaatgaggtccgtgtacagctc

SEQ ID NO: 117 - DNA, 17 bp, Artificial Sequence
  17nt guide, mutation at position 3 of seed region
gctgtacacggacctca

SEQ ID NO: 118 - DNA, 17 bp, Artificial Sequence
  17nt guide, mutation at position 3 of seed region
gctgtacacggacctca

SEQ ID NO: 119 - DNA, 22 bp, Artificial Sequence
  17nt guide, mutation at position 3 of seed region
caccggctgtacacggacctca

SEQ ID NO: 120 - DNA, 22 bp, Artificial Sequence
  17nt guide, mutation at position 3 of seed region
caaatgaggtccgtgtacagcc

SEQ ID NO: 121 - DNA, 16 bp, Artificial Sequence
  16nt guide, mutation at position 3 of seed region
ctgtacacggacctca

SEQ ID NO: 122 - DNA, 16 bp, Artificial Sequence
  16nt guide, mutation at position 3 of seed region
ctgtacacggacctca

SEQ ID NO: 123 - DNA, 21 bp, Artificial Sequence
  16nt guide, mutation at position 3 of seed region
caccgctgtacacggacctca

SEQ ID NO: 124 - DNA, 21 bp, Artificial Sequence
  16nt guide, mutation at position 3 of seed region
caaatgaggtccgtgtacagc

SEQ ID NO: 125 - DNA, 22 bp, Artificial Sequence
  22nt guide, mutation at position 7 in seed region
agagaatggagcagactcttgg

SEQ ID NO: 126 - DNA, 22 bp, Artificial Sequence
  22nt guide, mutation at position 7 in seed region
ccaagagtctgctccattctct

SEQ ID NO: 127 - DNA, 27 bp, Artificial Sequence
  22nt guide, mutation at position 7 in seed region
caccgccaagagtctgctccattctct

SEQ ID NO: 128 - DNA, 27 bp, Artificial Sequence
  22nt guide, mutation at position 7 in seed region
caaaagagaatggagcagactcttggc

SEQ ID NO: 129 - DNA, 21 bp, Artificial Sequence
  21nt guide, mutation at position 7 in seed region
agagaatggagcagactcttg

SEQ ID NO: 130 - DNA, 21 bp, Artificial Sequence
  21nt guide, mutation at position 7 in seed region
caagagtctgctccattctct

SEQ ID NO: 131 - DNA, 26 bp, Artificial Sequence
  21nt guide, mutation at position 7 in seed region
caccgcaagagtctgctccattctct

SEQ ID NO: 132 - DNA, 26 bp, Artificial Sequence
  21nt guide, mutation at position 7 in seed region
caaaagagaatggagcagactcttgc

SEQ ID NO: 133 - DNA, 20 bp, Artificial Sequence
  20nt guide, mutation at position 7 in seed region
agagaatggagcagactctt

SEQ ID NO: 134 - DNA, 20 bp, Artificial Sequence
  20nt guide, mutation at position 7 in seed region
aagagtctgctccattctct

SEQ ID NO: 135 - DNA, 25 bp, Artificial Sequence
  20nt guide, mutation at position 7 in seed region
caccgaagagtctgctccattctct

SEQ ID NO: 136 - DNA, 25 bp, Artificial Sequence
  20nt guide, mutation at position 7 in seed region
caaaagagaatggagcagactcttc

SEQ ID NO: 137 - DNA, 19 bp, Artificial Sequence
  19nt guide, mutation at position 7 in seed region
agagaatggagcagactct

SEQ ID NO: 138 - DNA, 19 bp, Artificial Sequence
  19nt guide, mutation at position 7 in seed region
agagtctgctccattctct

SEQ ID NO: 139 - DNA, 24 bp, Artificial Sequence
  19nt guide, mutation at position 7 in seed region
caccgagagtctgctccattctct

SEQ ID NO: 140 - DNA, 24 bp, Artificial Sequence
  19nt guide, mutation at position 7 in seed region
caaaagagaatggagcagactctc

SEQ ID NO: 141 - DNA, 18 bp, Artificial Sequence
  18nt guide, mutation at position 7 in seed region
agagaatggagcagactc

SEQ ID NO: 142 - DNA, 18 bp, Artificial Sequence
  18nt guide, mutation at position 7 in seed region
gagtctgctccattctct

SEQ ID NO: 143 - DNA, 23 bp, Artificial Sequence
  18nt guide, mutation at position 7 in seed region
caccggagtctgctccattctct

SEQ ID NO: 144 - DNA, 23 bp, Artificial Sequence
  18nt guide, mutation at position 7 in seed region
caaaagagaatggagcagactcc

SEQ ID NO: 145 - DNA, 17 bp, Artificial Sequence
  17nt guide, mutation at position 7 in seed region
agagaatggagcagact

SEQ ID NO: 146 - DNA, 17 bp, Artificial Sequence
  17nt guide, mutation at position 7 in seed region
agtctgctccattctct

SEQ ID NO: 147 - DNA, 22 bp, Artificial Sequence
  17nt guide, mutation at position 7 in seed region
caccgagtctgctccattctct

SEQ ID NO: 148 - DNA, 22 bp, Artificial Sequence
  17nt guide, mutation at position 7 in seed region
caaaagagaatggagcagactc

SEQ ID NO: 149 - DNA, 16 bp, Artificial Sequence
  16nt guide, mutation at position 7 in seed region
agagaatggagcagac

SEQ ID NO: 150 - DNA, 16 bp, Artificial Sequence
  16nt guide, mutation at position 7 in seed region
gtctgctccattctct

SEQ ID NO: 151 - DNA, 21 bp, Artificial Sequence
  16nt guide, mutation at position 7 in seed region
caccggtctgctccattctct

SEQ ID NO: 152 - DNA, 21 bp, Artificial Sequence
  16nt guide, mutation at position 7 in seed region
caaaagagaatggagcagacc

SEQ ID NO: 153 - DNA, 22 bp, Artificial Sequence
  22nt guide, mutation at position 8 of seed region
agagaacagagcagactcttgg

SEQ ID NO: 154 - DNA, 22 bp, Artificial Sequence
  22nt guide, mutation at position 8 of seed region
ccaagagtctgctctgttctct

SEQ ID NO: 155 - DNA, 27 bp, Artificial Sequence
  22nt guide, mutation at position 8 of seed region
caccgccaagagtctgctctgttctct

SEQ ID NO: 156 - DNA, 27 bp, Artificial Sequence
  22nt guide, mutation at position 8 of seed region
caaaagagaacagagcagactcttggc

SEQ ID NO: 157 - DNA, 21 bp, Artificial Sequence
  21nt guide, mutation at position 8 of seed region
agagaacagagcagactcttg

SEQ ID NO: 158 - DNA, 21 bp, Artificial Sequence
  21nt guide, mutation at position 8 of seed region
caagagtctgctctgttctct

SEQ ID NO: 159 - DNA, 26 bp, Artificial Sequence
  21nt guide, mutation at position 8 of seed region
caccgcaagagtctgctctgttctct

SEQ ID NO: 160 - DNA, 26 bp, Artificial Sequence
  21nt guide, mutation at position 8 of seed region
caaaagagaacagagcagactcttgc

SEQ ID NO: 161 - DNA, 20 bp, Artificial Sequence
  20nt guide, mutation at position 8 of seed region
agagaacagagcagactctt

SEQ ID NO: 162 - DNA, 20 bp, Artificial Sequence
  20nt guide, mutation at position 8 of seed region
aagagtctgctctgttctct

SEQ ID NO: 163 - DNA, 25 bp, Artificial Sequence
  20nt guide, mutation at position 8 of seed region
caccgaagagtctgctctgttctct

SEQ ID NO: 164 - DNA, 25 bp, Artificial Sequence
  20nt guide, mutation at position 8 of seed region
caaaagagaacagagcagactcttc

SEQ ID NO: 165 - DNA, 19 bp, Artificial Sequence
  19nt guide, mutation at position 8 of seed region
agagaacagagcagactct

SEQ ID NO: 166 - DNA, 19 bp, Artificial Sequence
  19nt guide, mutation at position 8 of seed region
agagtctgctctgttctct

SEQ ID NO: 167 - DNA, 24 bp, Artificial Sequence
  19nt guide, mutation at position 8 of seed region
caccgagagtctgctctgttctct

SEQ ID NO: 168 - DNA, 24 bp, Artificial Sequence
  19nt guide, mutation at position 8 of seed region
caaaagagaacagagcagactctc

SEQ ID NO: 169 - DNA, 18 bp, Artificial Sequence
  18nt guide, mutation at position 8 of seed region
agagaacagagcagactc

SEQ ID NO: 170 - DNA, 18 bp, Artificial Sequence
  18nt guide, mutation at position 8 of seed region
gagtctgctctgttctct

SEQ ID NO: 171 - DNA, 23 bp, Artificial Sequence
  18nt guide, mutation at position 8 of seed region
caccggagtctgctctgttctct

SEQ ID NO: 172 - DNA, 23 bp, Artificial Sequence
  18nt guide, mutation at position 8 of seed region
caaaagagaacagagcagactcc

SEQ ID NO: 173 - DNA, 17 bp, Artificial Sequence
  17nt guide, mutation at position 8 of seed region
agagaacagagcagact

SEQ ID NO: 174 - DNA, 17 bp, Artificial Sequence
  17nt guide, mutation at position 8 of seed region
agtctgctctgttctct

SEQ ID NO: 175 - DNA, 22 bp, Artificial Sequence
  17nt guide, mutation at position 8 of seed region
caccgagtctgctctgttctct

SEQ ID NO: 176 - DNA, 22 bp, Artificial Sequence
  17nt guide, mutation at position 8 of seed region
caaaagagaacagagcagactc

SEQ ID NO: 177 - DNA, 16 bp, Artificial Sequence
  16nt guide, mutation at position 8 of seed region
agagaacagagcagac

SEQ ID NO: 178 - DNA, 16 bp, Artificial Sequence
  16nt guide, mutation at position 8 of seed region
gtctgctctgttctct

SEQ ID NO: 179 - DNA, 21 bp, Artificial Sequence
  16nt guide, mutation at position 8 of seed region
caccggtctgctctgttctct

SEQ ID NO: 180 - DNA, 21 bp, Artificial Sequence
  16nt guide, mutation at position 8 of seed region
caaaagagaacagagcagacc

SEQ ID NO: 181 - DNA, 20 bp, Artificial Sequence
  Leu509Arg
gactgtcatggatgtccgga

SEQ ID NO: 182 - DNA, 20 bp, Artificial Sequence
  Leu509Arg
gactgtcatggatgtccgga

SEQ ID NO: 183 - DNA, 25 bp, Artificial Sequence
  Leu509Arg
caccggactgtcatggatgtccgga

SEQ ID NO: 184 - DNA, 25 bp, Artificial Sequence
  Leu509Arg
caaatccggacatccatgacagtcc

SEQ ID NO: 185 - DNA, 20 bp, Artificial Sequence
  Leu509Arg
tggggactgtcatggatgtc

SEQ ID NO: 186 - DNA, 20 bp, Artificial Sequence
  Leu509Arg
tggggactgtcatggatgtc

SEQ ID NO: 187 - DNA, 25 bp, Artificial Sequence
  Leu509Arg
caccgtggggactgtcatggatgtc

SEQ ID NO: 188 - DNA, 25 bp, Artificial Sequence
  Leu509Arg
caaagacatccatgacagtccccac

SEQ ID NO: 189 - DNA, 20 bp, Artificial Sequence
  Arg666Ser
gagctctgtgcgactaggtg

SEQ ID NO: 190 - DNA, 20 bp, Artificial Sequence
  Arg666Ser
cacctagtcgcacagagctc

SEQ ID NO: 191 - DNA, 25 bp, Artificial Sequence
  Arg666Ser
caccgcacctagtcgcacagagctc

SEQ ID NO: 192 - DNA, 25 bp, Artificial Sequence
  Arg666Ser
caaagagctctgtgcgactaggtgc

SEQ ID NO: 193 - DNA, 20 bp, Artificial Sequence
  Arg666Ser
ctagtcgcacagagctctgg

SEQ ID NO: 194 - DNA, 20 bp, Artificial Sequence
  Arg666Ser
ccagagctctgtgcgactag

SEQ ID NO: 195 - DNA, 25 bp, Artificial Sequence
  Arg666Ser
caccgccagagctctgtgcgactag

SEQ ID NO: 196 - DNA, 25 bp, Artificial Sequence
  Arg666Ser
caaactagtcgcacagagctctggc

SEQ ID NO: 197 - DNA, 20 bp, Artificial Sequence
  Gly623Asp
cctgacatcatgaccacaaa

SEQ ID NO: 198 - DNA, 20 bp, Artificial Sequence
  Gly623Asp
cctgacatcatgaccacaaa

SEQ ID NO: 199 - DNA, 25 bp, Artificial Sequence
  Gly623Asp
caccgcctgacatcatgaccacaaa

SEQ ID NO: 200 - DNA, 25 bp, Artificial Sequence
  Gly623Asp
caaatttgtggtcatgatgtcaggc

SEQ ID NO: 201 - DNA, 20 bp, Artificial Sequence
  Glu498Val
ggacgtggtgatcgccacct

SEQ ID NO: 202 - DNA, 20 bp, Artificial Sequence
  Glu498Val
aggtggcgatcaccacgtcc

SEQ ID NO: 203 - DNA, 25 bp, Artificial Sequence
  Glu498Val
caccgaggtggcgatcaccacgtcc

SEQ ID NO: 204 - DNA, 26 bp, Artificial Sequence
  Glu498Val
caaaggacgtggtgatcgccaccctc

SEQ ID NO: 205 - DNA, 20 bp, Artificial Sequence
  Arg503Pro
agctgctggagggcgaggag

SEQ ID NO: 206 - DNA, 20 bp, Artificial Sequence
  Arg503Pro
ctcctcgccctccagcagct

SEQ ID NO: 207 - DNA, 25 bp, Artificial Sequence
  Arg503Pro
caccgctcctcgccctccagcagct

SEQ ID NO: 208 - DNA, 25 bp, Artificial Sequence
  Arg503Pro
caaaagctgctggagggcgaggagc

SEQ ID NO: 209 - DNA, 20 bp, Artificial Sequence
  Arg503Pro
agctgctggagggcgaggag

SEQ ID NO: 210 - DNA, 20 bp, Artificial Sequence
  Arg503Pro
ctcctcgccctccagcagct

SEQ ID NO: 211 - DNA, 25 bp, Artificial Sequence
  Arg503Pro
caccgctcctcgccctccagcagct

SEQ ID NO: 212 - DNA, 25 bp, Artificial Sequence
  Arg503Pro
caaaagctgctggagggcgaggagc

SEQ ID NO: 213 - DNA, 20 bp, Artificial Sequence
  Arg503Pro
accccaagctgctggagggc

SEQ ID NO: 214 - DNA, 20 bp, Artificial Sequence
  Arg503Pro
gccctccagcagcttggggt

SEQ ID NO: 215 - DNA, 25 bp, Artificial Sequence
  Arg503Pro
caccggccctccagcagcttggggt

SEQ ID NO: 216 - DNA, 25 bp, Artificial Sequence
  Arg503Pro
caaaaccccaagctgctggagggcc

SEQ ID NO: 217 - DNA, 21 bp, Artificial Sequence
  Arg503Pro
taccccaagctgctggagggc

SEQ ID NO: 218 - DNA, 21 bp, Artificial Sequence
  Arg503Pro
taccccaagctgctggagggc

SEQ ID NO: 219 - DNA, 26 bp, Artificial Sequence
  Arg503Pro
caccgtaccccaagctgctggagggc

SEQ ID NO: 220 - DNA, 26 bp, Artificial Sequence
  Arg503Pro
caaagccctccagcagcttggggtac

SEQ ID NO: 221 - DNA, 20 bp, Artificial Sequence
  Glu509Lys
ccgcaagctgctggagggca

SEQ ID NO: 222 - DNA, 20 bp, Artificial Sequence
  Glu509Lys
ccgcaagctgctggagggcc

SEQ ID NO: 223 - DNA, 25 bp, Artificial Sequence
  Glu509Lys
caccgccgcaagctgctggagggcc

SEQ ID NO: 224 - DNA, 25 bp, Artificial Sequence
  Glu509Lys
caaaggccctccagcagcttgcggc

SEQ ID NO: 225 - DNA, 20 bp, Artificial Sequence
  Glu509Lys
ggccctccagcagcttgcgg

SEQ ID NO: 226 - DNA, 20 bp, Artificial Sequence
  Glu509Lys
ccgcaagctgctggagggcc

SEQ ID NO: 227 - DNA, 25 bp, Artificial Sequence
  Glu509Lys
caccgccgcaagctgctggagggcc

SEQ ID NO: 228 - DNA, 25 bp, Artificial Sequence
  Glu509Lys
caaaggccctccagcagcttgcggc

SEQ ID NO: 229 - DNA, 20 bp, Artificial Sequence
  Gln130Pro
aatcttaatgatagattagc

SEQ ID NO: 230 - DNA, 20 bp, Artificial Sequence
  Gln130Pro
gctaatctatcattaagatt

SEQ ID NO: 231 - DNA, 25 bp, Artificial Sequence
  Gln130Pro
caccggctaatctatcattaagatt

SEQ ID NO: 232 - DNA, 25 bp, Artificial Sequence
  Gln130Pro
caaaaatcttaatgatagattagcc

SEQ ID NO: 233 - DNA, 20 bp, Artificial Sequence
  Leu132Pro
atgatagattagcttcctac

SEQ ID NO: 234 - DNA, 20 bp, Artificial Sequence
  Leu132Pro
gtaggaagctaatctatcat

SEQ ID NO: 235 - DNA, 25 bp, Artificial Sequence
  Leu132Pro
caccggtaggaagctaatctatcat

SEQ ID NO: 236 - DNA, 25 bp, Artificial Sequence
  Leu132Pro
caaaatgatagattagcttcctacc

SEQ ID NO: 237 - DNA, 20 bp, Artificial Sequence
  Leu132Pro
atgatagattagcttcctac

SEQ ID NO: 238 - DNA, 20 bp, Artificial Sequence
  Leu132Pro
gtaggaagctaatctatcat

SEQ ID NO: 239 - DNA, 25 bp, Artificial Sequence
  Leu132Pro
caccggtaggaagctaatctatcat

SEQ ID NO: 240 - DNA, 25 bp, Artificial Sequence
  Leu132Pro
caaaatgatagattagcttcctacc

SEQ ID NO: 241 - DNA, 20 bp, Artificial Sequence
  Leu132Pro
aatgatagattagcttccta

SEQ ID NO: 242 - DNA, 20 bp, Artificial Sequence
  Leu132Pro
taggaagctaatctatcatt

SEQ ID NO: 243 - DNA, 25 bp, Artificial Sequence
  Leu132Pro
caccgtaggaagctaatctatcatt

SEQ ID NO: 244 - DNA, 25 bp, Artificial Sequence
  Leu132Pro
caaaaatgatagattagcttcctac

SEQ ID NO: 245 - DNA, 20 bp, Artificial Sequence
  Asn133Lys
aagaaactatgcaaaatctt

SEQ ID NO: 246 - DNA, 20 bp, Artificial Sequence
  Asn133Lys
aagaaactatgcaaaatctt

SEQ ID NO: 247 - DNA, 25 bp, Artificial Sequence
  Asn133Lys
caccgaagaaactatgcaaaatctt

SEQ ID NO: 248 - DNA, 25 bp, Artificial Sequence
  Asn133Lys
caaaaagattttgcatagtttcttc

SEQ ID NO: 249 - DNA, 20 bp, Artificial Sequence
  Asn133Lys
aagaaactatgcaaaatctt

SEQ ID NO: 250 - DNA, 20 bp, Artificial Sequence
  Asn133Lys
aagaaactatgcaaaatctt

SEQ ID NO: 251 - DNA, 25 bp, Artificial Sequence
  Asn133Lys
caccgaagaaactatgcaaaatctt

SEQ ID NO: 252 - DNA, 25 bp, Artificial Sequence
  Asn133Lys
caaaaagattttgcatagtttcttc

SEQ ID NO: 253 - DNA, 20 bp, Artificial Sequence
  Asn133Lys
agaaactatgcaaaatctta

SEQ ID NO: 254 - DNA, 20 bp, Artificial Sequence
  Asn133Lys
agaaactatgcaaaatctta

SEQ ID NO: 255 - DNA, 25 bp, Artificial Sequence
  Asn133Lys
caccgagaaactatgcaaaatctta

SEQ ID NO: 256 - DNA, 25 bp, Artificial Sequence
  Asn133Lys
caaataagattttgcatagtttctc

SEQ ID NO: 257 - DNA, 20 bp, Artificial Sequence
  Asn133Lys
ggatagattagcttcctacc

SEQ ID NO: 258 - DNA, 20 bp, Artificial Sequence
  Asn133Lys
ggatagattagcttcctacc

SEQ ID NO: 259 - DNA, 25 bp, Artificial Sequence
  Asn133Lys
caccgggatagattagcttcctacc

SEQ ID NO: 260 - DNA, 25 bp, Artificial Sequence
  Asn133Lys
caaaggtaggaagctaatctatccc

SEQ ID NO: 261 - DNA, 21 bp, Artificial Sequence
  Asn133Lys
aaggatagattagcttcctac

SEQ ID NO: 262 - DNA, 21 bp, Artificial Sequence
  Asn133Lys
aaggatagattagcttcctac

SEQ ID NO: 263 - DNA, 26 bp, Artificial Sequence
  Asn133Lys
caccgaaggatagattagcttcctac

SEQ ID NO: 264 - DNA, 26 bp, Artificial Sequence
  Asn133Lys
caaagtaggaagctaatctatccttc

SEQ ID NO: 265 - DNA, 20 bp, Artificial Sequence
  Arg135Gly
aactatgcaaaatcttaatg

SEQ ID NO: 266 - DNA, 20 bp, Artificial Sequence
  Arg135Gly
aactatgcaaaatcttaatg

SEQ ID NO: 267 - DNA, 25 bp, Artificial Sequence
  Arg135Gly
caccgaactatgcaaaatcttaatg

SEQ ID NO: 268 - DNA, 25 bp, Artificial Sequence
  Arg135Gly
caaacattaagattttgcatagttc

SEQ ID NO: 269 - DNA, 20 bp, Artificial Sequence
  Arg135Gly
actatgcaaaatcttaatga

SEQ ID NO: 270 - DNA, 20 bp, Artificial Sequence
  Arg135Gly
actatgcaaaatcttaatga

SEQ ID NO: 271 - DNA, 25 bp, Artificial Sequence
  Arg135Gly
caccgactatgcaaaatcttaatga

SEQ ID NO: 272 - DNA, 25 bp, Artificial Sequence
  Arg135Gly
caaatcattaagattttgcatagtc

SEQ ID NO: 273 - DNA, 20 bp, Artificial Sequence
  Arg135Gly
ggtaggaagctaatccatca

SEQ ID NO: 274 - DNA, 20 bp, Artificial Sequence
  Arg135Gly
tgatggattagcttcctacc

SEQ ID NO: 275 - DNA, 25 bp, Artificial Sequence
  Arg135Gly
caccgtgatggattagcttcctacc

SEQ ID NO: 276 - DNA, 25 bp, Artificial Sequence
  Arg135Gly
caaaggtaggaagctaatccatcac

SEQ ID NO: 277 - DNA, 21 bp, Artificial Sequence
  Arg135Gly
aatgatggattagcttcctac

SEQ ID NO: 278 - DNA, 21 bp, Artificial Sequence
  Arg135Gly
aatgatggattagcttcctac

SEQ ID NO: 279 - DNA, 26 bp, Artificial Sequence
  Arg135Gly
caccgaatgatggattagcttcctac

SEQ ID NO: 280 - DNA, 26 bp, Artificial Sequence
  Arg135Gly
caaagtaggaagctaatccatcattc

SEQ ID NO: 281 - DNA, 20 bp, Artificial Sequence
  Arg135Ile
tgatatattagcttcctacc

SEQ ID NO: 282 - DNA, 20 bp, Artificial Sequence
  Arg135Ile
tgatatattagcttcctacc

SEQ ID NO: 283 - DNA, 25 bp, Artificial Sequence
  Arg135Ile
caccgtgatatattagcttcctacc

SEQ ID NO: 284 - DNA, 25 bp, Artificial Sequence
  Arg135Ile
caaaggtaggaagctaatatatcac

SEQ ID NO: 285 - DNA, 21 bp, Artificial Sequence
  Arg135Ile
aatgatatattagcttcctac

SEQ ID NO: 286 - DNA, 21 bp, Artificial Sequence
  Arg135Ile
aatgatatattagcttcctac

SEQ ID NO: 287 - DNA, 26 bp, Artificial Sequence
  Arg135Ile
caccgaatgatatattagcttcctac

SEQ ID NO: 288 - DNA, 26 bp, Artificial Sequence
  Arg135Ile
caaagtaggaagctaatatatcattc

SEQ ID NO: 289 - DNA, 20 bp, Artificial Sequence
  Arg135Thr
tgatacattagcttcctacc

SEQ ID NO: 290 - DNA, 20 bp, Artificial Sequence
  Arg135Thr
tgatacattagcttcctacc

SEQ ID NO: 291 - DNA, 25 bp, Artificial Sequence
  Arg135Thr
caccgtgatacattagcttcctacc

SEQ ID NO: 292 - DNA, 25 bp, Artificial Sequence
  Arg135Thr
caaaggtaggaagctaatgtatcac

SEQ ID NO: 293 - DNA, 21 bp, Artificial Sequence
  Arg135Thr
aatgatacattagcttcctac

SEQ ID NO: 294 - DNA, 21 bp, Artificial Sequence
  Arg135Thr
aatgatacattagcttcctac

SEQ ID NO: 295 - DNA, 26 bp, Artificial Sequence
  Arg135Thr
caccgaatgatacattagcttcctac

SEQ ID NO: 296 - DNA, 26 bp, Artificial Sequence
  Arg135Thr
caaagtaggaagctaatgtatcattc

SEQ ID NO: 297 - DNA, 20 bp, Artificial Sequence
  Arg135Ser
tgatagcttagcttcctacc

SEQ ID NO: 298 - DNA, 20 bp, Artificial Sequence
  Arg135Ser
tgatagcttagcttcctacc

SEQ ID NO: 299 - DNA, 25 bp, Artificial Sequence
  Arg135Ser
caccgtgatagcttagcttcctacc

SEQ ID NO: 300 - DNA, 25 bp, Artificial Sequence
  Arg135Ser
caaaggtaggaagctaagctatcac

SEQ ID NO: 301 - DNA, 20 bp, Artificial Sequence
  Arg135Ser
atgatagcttagcttcctac

SEQ ID NO: 302 - DNA, 20 bp, Artificial Sequence
  Arg135Ser
atgatagcttagcttcctac

SEQ ID NO: 303 - DNA, 25 bp, Artificial Sequence
  Arg135Ser
caccgatgatagcttagcttcctac

SEQ ID NO: 304 - DNA, 25 bp, Artificial Sequence
  Arg135Ser
caaagtaggaagctaagctatcatc

SEQ ID NO: 305 - DNA, 20 bp, Artificial Sequence
  Ala137Pro
tcctacctggataaggtgcg

SEQ ID NO: 306 - DNA, 20 bp, Artificial Sequence
  Ala137Pro
cgcaccttatccaggtagga

SEQ ID NO: 307 - DNA, 25 bp, Artificial Sequence
  Ala137Pro
caccgcgcaccttatccaggtagga

SEQ ID NO: 308 - DNA, 25 bp, Artificial Sequence
  Ala137Pro
caaatcctacctggataaggtgcgc

SEQ ID NO: 309 - DNA, 20 bp, Artificial Sequence
  Ala137Pro
tgatagattaccttcctacc

SEQ ID NO: 310 - DNA, 20 bp, Artificial Sequence
  Ala137Pro
tgatagattaccttcctacc

SEQ ID NO: 311 - DNA, 25 bp, Artificial Sequence
  Ala137Pro
caccgtgatagattaccttcctacc

SEQ ID NO: 312 - DNA, 25 bp, Artificial Sequence
  Ala137Pro
caaaggtaggaaggtaatctatcac

SEQ ID NO: 313 - DNA, 21 bp, Artificial Sequence
  Ala137Pro
aatgatagattaccttcctac

SEQ ID NO: 314 - DNA, 21 bp, Artificial Sequence
  Ala137Pro
aatgatagattaccttcctac

SEQ ID NO: 315 - DNA, 26 bp, Artificial Sequence
  Ala137Pro
caccgaatgatagattaccttcctac

SEQ ID NO: 316 - DNA, 26 bp, Artificial Sequence
  Ala137Pro
caaagtaggaaggtaatctatcattc

SEQ ID NO: 317 - DNA, 20 bp, Artificial Sequence
  Leu140Arg
atgatagattagcttcctac

SEQ ID NO: 318 - DNA, 20 bp, Artificial Sequence
  Leu140Arg
atgatagattagcttcctac

SEQ ID NO: 319 - DNA, 25 bp, Artificial Sequence
  Leu140Arg
caccgatgatagattagcttcctac

SEQ ID NO: 320 - DNA, 25 bp, Artificial Sequence
  Leu140Arg
caaaatgatagattagcttcctacc

SEQ ID NO: 321 - DNA, 20 bp, Artificial Sequence
  Leu140Arg
agctcgcaccttatcccggt

SEQ ID NO: 322 - DNA, 20 bp, Artificial Sequence
  Leu140Arg
agctcgcaccttatcccggt

SEQ ID NO: 323 - DNA, 25 bp, Artificial Sequence
  Leu140Arg
caccgagctcgcaccttatcccggt

SEQ ID NO: 324 - DNA, 25 bp, Artificial Sequence
  Leu140Arg
caaaaccgggataaggtgcgagctc

SEQ ID NO: 325 - DNA, 20 bp, Artificial Sequence
  Val143Leu
ggataagttgcgagctctag

SEQ ID NO: 326 - DNA, 20 bp, Artificial Sequence
  Val143Leu
ctagagctcgcaacttatcc

SEQ ID NO: 327 - DNA, 25 bp, Artificial Sequence
  Val143Leu
caccgctagagctcgcaacttatcc

SEQ ID NO: 328 - DNA, 25 bp, Artificial Sequence
  Val143Leu
caaaggataagttgcgagctctagc

SEQ ID NO: 329 - DNA, 20 bp, Artificial Sequence
  Val143Leu
ggataagctgcgagctctag

SEQ ID NO: 330 - DNA, 20 bp, Artificial Sequence
  Val143Leu
ctagagctcgcagcttatcc

SEQ ID NO: 331 - DNA, 25 bp, Artificial Sequence
  Val143Leu
caccgctagagctcgcagcttatcc

SEQ ID NO: 332 - DNA, 25 bp, Artificial Sequence
  Val143Leu
caaaggataagctgcgagctctagc

SEQ ID NO: 333 - DNA, 20 bp, Artificial Sequence
  Lle391_Leu399dup
gcacagctgcatcagcaacc

SEQ ID NO: 334 - DNA, 20 bp, Artificial Sequence
  Lle391_Leu399dup
gcacagctgcatcagcaacc

SEQ ID NO: 335 - DNA, 25 bp, Artificial Sequence
  Lle391_Leu399dup
caccggcacagctgcatcagcaacc

SEQ ID NO: 336 - DNA, 25 bp, Artificial Sequence
  Lle391_Leu399dup
caaaggttgctgatgcagctgtgcc

SEQ ID NO: 337 - DNA, 20 bp, Artificial Sequence
  Ile 426Val
ggagctggagagtgagacct

SEQ ID NO: 338 - DNA, 20 bp, Artificial Sequence
  Ile 426Val
aggtctcactctccagctcc

SEQ ID NO: 339 - DNA, 25 bp, Artificial Sequence
  Ile 426Val
caccgaggtctcactctccagctcc

SEQ ID NO: 340 - DNA, 25 bp, Artificial Sequence
  Ile 426Val
caaaggagctggagagtgagacctc

SEQ ID NO: 341 - DNA, 20 bp, Artificial Sequence
  Ile 426Val
tcaaggcccgcctggagctg

SEQ ID NO: 342 - DNA, 20 bp, Artificial Sequence
  Ile 426Val
tcaaggcccgcctggagctg

SEQ ID NO: 343 - DNA, 25 bp, Artificial Sequence
  Ile 426Val
caccgtcaaggcccgcctggagctg

SEQ ID NO: 344 - DNA, 25 bp, Artificial Sequence
  Ile 426Val
caaacagctccaggcgggccttgac

SEQ ID NO: 345 - DNA, 20 bp, Artificial Sequence
  Ile 426Ser
caaggcccgcctggagctgg

SEQ ID NO: 346 - DNA, 20 bp, Artificial Sequence
  Ile 426Ser
caaggcccgcctggagctgg

SEQ ID NO: 347 - DNA, 25 bp, Artificial Sequence
  Ile 426Ser
caccgcaaggcccgcctggagctgg

SEQ ID NO: 348 - DNA, 25 bp, Artificial Sequence
  Ile 426Ser
caaaccagctccaggcgggccttgc

SEQ ID NO: 349 - DNA, 20 bp, Artificial Sequence
  Ile 426Ser
ggagctggaggttgagacct

SEQ ID NO: 350 - DNA, 20 bp, Artificial Sequence
  Ile 426Ser
aggtctcaacctccagctcc

SEQ ID NO: 351 - DNA, 25 bp, Artificial Sequence
  Ile 426Ser
caccgaggtctcaacctccagctcc

SEQ ID NO: 352 - DNA, 25 bp, Artificial Sequence
  Ile 426Ser
caaaggagctggaggttgagacctc

SEQ ID NO: 353 - DNA, 20 bp, Artificial Sequence
  Tyr429Asp
ggagctggagattgagaccg

SEQ ID NO: 354 - DNA, 20 bp, Artificial Sequence
  Tyr429Asp
cggtctcaatctccagctcc

SEQ ID NO: 355 - DNA, 25 bp, Artificial Sequence
  Tyr429Asp
caccgcggtctcaatctccagctcc

SEQ ID NO: 356 - DNA, 25 bp, Artificial Sequence
  Tyr429Asp
caaaggagctggagattgagaccgc

SEQ ID NO: 357 - DNA, 20 bp, Artificial Sequence
  Tyr429Cys
gccgccgcctgctggacggg

SEQ ID NO: 358 - DNA, 20 bp, Artificial Sequence
  Tyr429Cys
cccgtccagcaggcggcggc

SEQ ID NO: 359 - DNA, 25 bp, Artificial Sequence
  Tyr429Cys
caccgcccgtccagcaggcggcggc

SEQ ID NO: 360 - DNA, 25 bp, Artificial Sequence
  Tyr429Cys
caaagccgccgcctgctggacgggc

SEQ ID NO: 361 - DNA, 20 bp, Artificial Sequence
  Arg430Pro
gcctgctggacggggaggcc

SEQ ID NO: 362 - DNA, 20 bp, Artificial Sequence
  Arg430Pro
ggcctccccgtccagcaggc

SEQ ID NO: 363 - DNA, 25 bp, Artificial Sequence
  Arg430Pro
caccgggcctccccgtccagcaggc

SEQ ID NO: 364 - DNA, 25 bp, Artificial Sequence
  Arg430Pro
caaagcctgctggacggggaggccc

SEQ ID NO: 365 - DNA, 20 bp, Artificial Sequence
  Arg430Pro
gcctgctggacggggaggcc

SEQ ID NO: 366 - DNA, 20 bp, Artificial Sequence
  Arg430Pro
ggcctccccgtccagcaggc

SEQ ID NO: 367 - DNA, 25 bp, Artificial Sequence
  Arg430Pro
caccgggcctccccgtccagcaggc

SEQ ID NO: 368 - DNA, 25 bp, Artificial Sequence
  Arg430Pro
caaagcctgctggacggggaggccc

SEQ ID NO: 369 - DNA, 18 bp, Artificial Sequence
  Arg430Pro
cgcctgctggacggggag

SEQ ID NO: 370 - DNA, 18 bp, Artificial Sequence
  Arg430Pro
ctccccgtccagcaggcg

SEQ ID NO: 371 - DNA, 23 bp, Artificial Sequence
  Arg430Pro
caccgctccccgtccagcaggcg

SEQ ID NO: 372 - DNA, 23 bp, Artificial Sequence
  Arg430Pro
caaacgcctgctggacggggagc

SEQ ID NO: 373 - DNA, 20 bp, Artificial Sequence
  Arg430Pro
acccccgcctgctggacggg

SEQ ID NO: 374 - DNA, 20 bp, Artificial Sequence
  Arg430Pro
cccgtccagcaggcgggggt

SEQ ID NO: 375 - DNA, 25 bp, Artificial Sequence
  Arg430Pro
caccgcccgtccagcaggcgggggt

SEQ ID NO: 376 - DNA, 25 bp, Artificial Sequence
  Arg430Pro
caaaacccccgcctgctggacgggc

SEQ ID NO: 377 - DNA, 20 bp, Artificial Sequence
  Leu433Arg
ttgagacctaccgccgcctg

SEQ ID NO: 378 - DNA, 20 bp, Artificial Sequence
  Leu433Arg
ttgagacctaccgccgcctg

SEQ ID NO: 379 - DNA, 25 bp, Artificial Sequence
  Leu433Arg
caccgttgagacctaccgccgcctg

SEQ ID NO: 380 - DNA, 25 bp, Artificial Sequence
  Leu433Arg
caaacaggcggcggtaggtctcaac

SEQ ID NO: 381 - DNA, 20 bp, Artificial Sequence
  Leu433Arg
gcgggacggggaggcccaag

SEQ ID NO: 382 - DNA, 20 bp, Artificial Sequence
  Leu433Arg
cttgggcctccccgtcccgc

SEQ ID NO: 383 - DNA, 25 bp, Artificial Sequence
  Leu433Arg
caccgcttgggcctccccgtcccgc

SEQ ID NO: 384 - DNA, 25 bp, Artificial Sequence
  Leu433Arg
caaagcgggacggggaggcccaagc

SEQ ID NO: 385 - DNA, 21 bp, Artificial Sequence
  Leu433Arg
gcctgcgggacggggaggccc

SEQ ID NO: 386 - DNA, 21 bp, Artificial Sequence
  Leu433Arg
gcctgcgggacggggaggccc

SEQ ID NO: 387 - DNA, 26 bp, Artificial Sequence
  Leu433Arg
caccggcctgcgggacggggaggccc

SEQ ID NO: 388 - DNA, 26 bp, Artificial Sequence
  Leu433Arg
caaagggcctccccgtcccgcaggcc

SEQ ID NO: 389 - DNA, 20 bp, Artificial Sequence
  Arg555Gln
agagaacagagcagactctt

SEQ ID NO: 390 - DNA, 20 bp, Artificial Sequence
  Arg555Gln
aagagtctgctctgttctct

SEQ ID NO: 391 - DNA, 25 bp, Artificial Sequence
  Arg555Gln
caccgaagagtctgctctgttctct

SEQ ID NO: 392 - DNA, 25 bp, Artificial Sequence
  Arg555Gln
caaaagagaacagagcagactcttc

SEQ ID NO: 393 - DNA, 21 bp, Artificial Sequence
  Arg555Gln
ccaagagaacagagcagactc

SEQ ID NO: 394 - DNA, 21 bp, Artificial Sequence
  Arg555Gln
ccaagagaacagagcagactc

SEQ ID NO: 395 - DNA, 26 bp, Artificial Sequence
  Arg555Gln
caccgccaagagaacagagcagactc

SEQ ID NO: 396 - DNA, 26 bp, Artificial Sequence
  Arg555Gln
caaagagtctgctctgttctcttggc

SEQ ID NO: 397 - DNA, 20 bp, Artificial Sequence
  Arg124Cys
tcagctgtacacggactgca

SEQ ID NO: 398 - DNA, 20 bp, Artificial Sequence
  Arg124Cys
tcagctgtacacggactgca

SEQ ID NO: 399 - DNA, 25 bp, Artificial Sequence
  Arg124Cys
caccgtcagctgtacacggactgca

SEQ ID NO: 400 - DNA, 25 bp, Artificial Sequence
  Arg124Cys
caaatgcagtccgtgtacagctgac

SEQ ID NO: 401 - DNA, 22 bp, Artificial Sequence
  Arg124Cys
ctgtacacggactgcacggaga

SEQ ID NO: 402 - DNA, 22 bp, Artificial Sequence
  Arg124Cys
tctccgtgcagtccgtgtacag

SEQ ID NO: 403 - DNA, 27 bp, Artificial Sequence
  Arg124Cys
caccgtctccgtgcagtccgtgtacag

SEQ ID NO: 404 - DNA, 27 bp, Artificial Sequence
  Arg124Cys
caaactgtacacggactgcacggagac

SEQ ID NO: 405 - DNA, 20 bp, Artificial Sequence
  Val505Asp
ccccccaatggggactgaca

SEQ ID NO: 406 - DNA, 20 bp, Artificial Sequence
  Val505Asp
ccccccaatggggactgaca

SEQ ID NO: 407 - DNA, 25 bp, Artificial Sequence
  Val505Asp
caccgccccccaatggggactgaca

SEQ ID NO: 408 - DNA, 25 bp, Artificial Sequence
  Val505Asp
caaatgtcagtccccattggggggc

SEQ ID NO: 409 - DNA, 20 bp, Artificial Sequence
  Val505Asp
cccccccaatggggactgac

SEQ ID NO: 410 - DNA, 20 bp, Artificial Sequence
  Val505Asp
cccccccaatggggactgac

SEQ ID NO: 411 - DNA, 25 bp, Artificial Sequence
  Val505Asp
caccgcccccccaatggggactgac

SEQ ID NO: 412 - DNA, 25 bp, Artificial Sequence
  Val505Asp
caaagtcagtccccattgggggggc

SEQ ID NO: 413 - DNA, 20 bp, Artificial Sequence
  Ile522Asn
accagtctgcaggactgacg

SEQ ID NO: 414 - DNA, 20 bp, Artificial Sequence
  Ile522Asn
cgtcagtcctgcagactggt

SEQ ID NO: 415 - DNA, 25 bp, Artificial Sequence
  Ile522Asn
caccgcgtcagtcctgcagactggt

SEQ ID NO: 416 - DNA, 25 bp, Artificial Sequence
  Ile522Asn
caaaaccagtctgcaggactgacgc

SEQ ID NO: 417 - DNA, 20 bp, Artificial Sequence
  Leu569Arg
ccaaggaacttgccaacatc

SEQ ID NO: 418 - DNA, 20 bp, Artificial Sequence
  Leu569Arg
ccaaggaacttgccaacatc

SEQ ID NO: 419 - DNA, 25 bp, Artificial Sequence
  Leu569Arg
caccgccaaggaacttgccaacatc

SEQ ID NO: 420 - DNA, 25 bp, Artificial Sequence
  Leu569Arg
caaagatgttggcaagttccttggc

SEQ ID NO: 421 - DNA, 20 bp, Artificial Sequence
  Leu569Arg
acatccggaaataccacatt

SEQ ID NO: 422 - DNA, 20 bp, Artificial Sequence
  Leu569Arg
aatgtggtatttccggatgt

SEQ ID NO: 423 - DNA, 25 bp, Artificial Sequence
  Leu569Arg
caccgaatgtggtatttccggatgt

SEQ ID NO: 424 - DNA, 25 bp, Artificial Sequence
  Leu569Arg
caaaacatccggaaataccacattc

SEQ ID NO: 425 - DNA, 20 bp, Artificial Sequence
  His572Arg
aacatcctgaaataccgcat

SEQ ID NO: 426 - DNA, 20 bp, Artificial Sequence
  His572Arg
aacatcctgaaataccgcat

SEQ ID NO: 427 - DNA, 25 bp, Artificial Sequence
  His572Arg
caccgaacatcctgaaataccgcat

SEQ ID NO: 428 - DNA, 25 bp, Artificial Sequence
  His572Arg
caaaatgcggtatttcaggatgttc

SEQ ID NO: 429 - DNA, 21 bp, Artificial Sequence
  His572Arg
aaataccgcattggtgatgaa

SEQ ID NO: 430 - DNA, 21 bp, Artificial Sequence
  His572Arg
ttcatcaccaatgcggtattt

SEQ ID NO: 431 - DNA, 26 bp, Artificial Sequence
  His572Arg
caccgttcatcaccaatgcggtattt

SEQ ID NO: 432 - DNA, 26 bp, Artificial Sequence
  His572Arg
caaaaaataccgcattggtgatgaac

SEQ ID NO: 433 - DNA, 20 bp, Artificial Sequence
  Asp214Tyr
caatggcaactgcttcatcc

SEQ ID NO: 434 - DNA, 20 bp, Artificial Sequence
  Asp214Tyr
caatggcaactgcttcatcc

SEQ ID NO: 435 - DNA, 25 bp, Artificial Sequence
  Asp214Tyr
caccgcaatggcaactgcttcatcc

SEQ ID NO: 436 - DNA, 25 bp, Artificial Sequence
  Asp214Tyr
caaaggatgaagcagttgccattgc

SEQ ID NO: 437 - DNA, 20 bp, Artificial Sequence
  Asp214Tyr
caatggctactgcttcatcc

SEQ ID NO: 438 - DNA, 20 bp, Artificial Sequence
  Asp214Tyr
caatggctactgcttcatcc

SEQ ID NO: 439 - DNA, 25 bp, Artificial Sequence
  Asp214Tyr
caccgcaatggctactgcttcatcc

SEQ ID NO: 440 - DNA, 25 bp, Artificial Sequence
  Asp214Tyr
caaaggatgaagcagtagccattgc

SEQ ID NO: 441 - DNA, 20 bp, Artificial Sequence
  Arg496Trp
gaccctgttcacgatggact

SEQ ID NO: 442 - DNA, 20 bp, Artificial Sequence
  Arg496Trp
gaccctgttcacgatggact

SEQ ID NO: 443 - DNA, 25 bp, Artificial Sequence
  Arg496Trp
caccggaccctgttcacgatggact

SEQ ID NO: 444 - DNA, 25 bp, Artificial Sequence
  Arg496Trp
caaaagtccatcgtgaacagggtcc

SEQ ID NO: 445 - DNA, 23 bp, Artificial Sequence
  Pro501Thr
acaatggggactgtcatggatgt

SEQ ID NO: 446 - DNA, 23 bp, Artificial Sequence
  Pro501Thr
acatccatgacagtccccattgt

SEQ ID NO: 447 - DNA, 28 bp, Artificial Sequence
  Pro501Thr
caccgacatccatgacagtccccattgt

SEQ ID NO: 448 - DNA, 28 bp, Artificial Sequence
  Pro501Thr
caaaacaatggggactgtcatggatgtc

SEQ ID NO: 449 - DNA, 20 bp, Artificial Sequence
  Arg514Pro
tttaggtaattagttccatc

SEQ ID NO: 450 - DNA, 20 bp, Artificial Sequence
  Arg514Pro
gatggaactaattacctaaa

SEQ ID NO: 451 - DNA, 25 bp, Artificial Sequence
  Arg514Pro
caccggatggaactaattacctaaa

SEQ ID NO: 452 - DNA, 25 bp, Artificial Sequence
  Arg514Pro
caaatttaggtaattagttccatcc

SEQ ID NO: 453 - DNA, 20 bp, Artificial Sequence
  Arg514Pro
tgaagggagacaatcccttt

SEQ ID NO: 454 - DNA, 20 bp, Artificial Sequence
  Arg514Pro
tgaagggagacaatcccttt

SEQ ID NO: 455 - DNA, 25 bp, Artificial Sequence
  Arg514Pro
caccgtgaagggagacaatcccttt

SEQ ID NO: 456 - DNA, 25 bp, Artificial Sequence
  Arg514Pro
caaaaaagggattgtctcccttcac

SEQ ID NO: 457 - DNA, 20 bp, Artificial Sequence
  Phe515Leu
tgaagggagacaatcgctta

SEQ ID NO: 458 - DNA, 20 bp, Artificial Sequence
  Phe515Leu
tgaagggagacaatcgctta

SEQ ID NO: 459 - DNA, 25 bp, Artificial Sequence
  Phe515Leu
caccgtgaagggagacaatcgctta

SEQ ID NO: 460 - DNA, 25 bp, Artificial Sequence
  Phe515Leu
caaataagcgattgtctcccttcac

SEQ ID NO: 461 - DNA, 21 bp, Artificial Sequence
  Phe515Leu
ttaaggtaattagttccatcc

SEQ ID NO: 462 - DNA, 21 bp, Artificial Sequence
  Phe515Leu
ttaaggtaattagttccatcc

SEQ ID NO: 463 - DNA, 26 bp, Artificial Sequence
  Phe515Leu
caccgttaaggtaattagttccatcc

SEQ ID NO: 464 - DNA, 26 bp, Artificial Sequence
  Phe515Leu
caaaggatggaactaattaccttaac

SEQ ID NO: 465 - DNA, 20 bp, Artificial Sequence
  Leu518Pro
gtagctgccatccagtctgc

SEQ ID NO: 466 - DNA, 20 bp, Artificial Sequence
  Leu518Pro
gcagactggatggcagctac

SEQ ID NO: 467 - DNA, 25 bp, Artificial Sequence
  Leu518Pro
caccggcagactggatggcagctac

SEQ ID NO: 468 - DNA, 25 bp, Artificial Sequence
  Leu518Pro
caaagtagctgccatccagtctgcc

SEQ ID NO: 469 - DNA, 20 bp, Artificial Sequence
  Leu518Arg
tgtgtgtgtatctacagcat

SEQ ID NO: 470 - DNA, 20 bp, Artificial Sequence
  Leu518Arg
tgtgtgtgtatctacagcat

SEQ ID NO: 471 - DNA, 25 bp, Artificial Sequence
  Leu518Arg
caccgtgtgtgtgtatctacagcat

SEQ ID NO: 472 - DNA, 25 bp, Artificial Sequence
  Leu518Arg
caaaatgctgtagatacacacacac

SEQ ID NO: 473 - DNA, 20 bp, Artificial Sequence
  Leu527Arg
ctgccatccagtctgcagga

SEQ ID NO: 474 - DNA, 20 bp, Artificial Sequence
  Leu527Arg
ctgccatccagtctgcagga

SEQ ID NO: 475 - DNA, 25 bp, Artificial Sequence
  Leu527Arg
caccgctgccatccagtctgcagga

SEQ ID NO: 476 - DNA, 25 bp, Artificial Sequence
  Leu527Arg
caaatcctgcagactggatggcagc

SEQ ID NO: 477 - DNA, 20 bp, Artificial Sequence
  Leu527Arg
catccagtctgcaggacgga

SEQ ID NO: 478 - DNA, 20 bp, Artificial Sequence
  Leu527Arg
catccagtctgcaggacgga

SEQ ID NO: 479 - DNA, 25 bp, Artificial Sequence
  Leu527Arg
caccgcatccagtctgcaggacgga

SEQ ID NO: 480 - DNA, 25 bp, Artificial Sequence
  Leu527Arg
caaatccgtcctgcagactggatgc

SEQ ID NO: 481 - DNA, 21 bp, Artificial Sequence
  Leu527Arg
tctgcaggacggacggagacc

SEQ ID NO: 482 - DNA, 21 bp, Artificial Sequence
  Leu527Arg
ggtctccgtccgtcctgcaga

SEQ ID NO: 483 - DNA, 26 bp, Artificial Sequence
  Leu527Arg
caccgggtctccgtccgtcctgcaga

SEQ ID NO: 484 - DNA, 26 bp, Artificial Sequence
  Leu527Arg
caaatctgcaggacggacggagaccc

SEQ ID NO: 485 - DNA, 20 bp, Artificial Sequence
  Thr538Pro
agtctttgctcccacaaatg

SEQ ID NO: 486 - DNA, 20 bp, Artificial Sequence
  Thr538Pro
catttgtgggagcaaagact

SEQ ID NO: 487 - DNA, 25 bp, Artificial Sequence
  Thr538Pro
caccgcatttgtgggagcaaagact

SEQ ID NO: 488 - DNA, 25 bp, Artificial Sequence
  Thr538Pro
caaaagtctttgctcccacaaatgc

SEQ ID NO: 489 - DNA, 20 bp, Artificial Sequence
  Thr538Pro
ggaaggagtctacccagtct

SEQ ID NO: 490 - DNA, 20 bp, Artificial Sequence
  Thr538Pro
agactgggtagactccttcc

SEQ ID NO: 491 - DNA, 25 bp, Artificial Sequence
  Thr538Pro
caccgagactgggtagactccttcc

SEQ ID NO: 492 - DNA, 25 bp, Artificial Sequence
  Thr538Pro
caaaggaaggagtctacccagtctc

SEQ ID NO: 493 - DNA, 21 bp, Artificial Sequence
  Thr538Pro
aaccgggaaggagtctaccca

SEQ ID NO: 494 - DNA, 21 bp, Artificial Sequence
  Thr538Pro
tgggtagactccttcccggtt

SEQ ID NO: 495 - DNA, 26 bp, Artificial Sequence
  Thr538Pro
caccgtgggtagactccttcccggtt

SEQ ID NO: 496 - DNA, 26 bp, Artificial Sequence
  Thr538Pro
caaaaaccgggaaggagtctacccac

SEQ ID NO: 497 - DNA, 20 bp, Artificial Sequence
  Thr538Arg
ctttgctcccacaaatgaag

SEQ ID NO: 498 - DNA, 20 bp, Artificial Sequence
  Thr538Arg
cttcatttgtgggagcaaag

SEQ ID NO: 499 - DNA, 25 bp, Artificial Sequence
  Thr538Arg
caccgcttcatttgtgggagcaaag

SEQ ID NO: 500 - DNA, 25 bp, Artificial Sequence
  Thr538Arg
caaactttgctcccacaaatgaagc

SEQ ID NO: 501 - DNA, 20 bp, Artificial Sequence
  Thr538Arg
ggaaggagtctacagagtct

SEQ ID NO: 502 - DNA, 20 bp, Artificial Sequence
  Thr538Arg
agactctgtagactccttcc

SEQ ID NO: 503 - DNA, 25 bp, Artificial Sequence
  Thr538Arg
caccgagactctgtagactccttcc

SEQ ID NO: 504 - DNA, 25 bp, Artificial Sequence
  Thr538Arg
caaaggaaggagtctacagagtctc

SEQ ID NO: 505 - DNA, 21 bp, Artificial Sequence
  Thr538Arg
aaccgggaaggagtctacaga

SEQ ID NO: 506 - DNA, 21 bp, Artificial Sequence
  Thr538Arg
tctgtagactccttcccggtt

SEQ ID NO: 507 - DNA, 26 bp, Artificial Sequence
  Thr538Arg
caccgtctgtagactccttcccggtt

SEQ ID NO: 508 - DNA, 26 bp, Artificial Sequence
  Thr538Arg
caaaaaccgggaaggagtctacagac

SEQ ID NO: 509 - DNA, 20 bp, Artificial Sequence
  Val539Asp
ggaaggagtctacacagact

SEQ ID NO: 510 - DNA, 20 bp, Artificial Sequence
  Val539Asp
agtctgtgtagactccttcc

SEQ ID NO: 511 - DNA, 25 bp, Artificial Sequence
  Val539Asp
caccgagtctgtgtagactccttcc

SEQ ID NO: 512 - DNA, 25 bp, Artificial Sequence
  Val539Asp
caaaggaaggagtctacacagactc

SEQ ID NO: 513 - DNA, 20 bp, Artificial Sequence
  Phe540del
ggaaggagtctacacagtcg

SEQ ID NO: 514 - DNA, 20 bp, Artificial Sequence
  Phe540del
cgactgtgtagactccttcc

SEQ ID NO: 515 - DNA, 25 bp, Artificial Sequence
  Phe540del
caccgcgactgtgtagactccttcc

SEQ ID NO: 516 - DNA, 25 bp, Artificial Sequence
  Phe540del
caaaggaaggagtctacacagtcgc

SEQ ID NO: 517 - DNA, 20 bp, Artificial Sequence
  Asn544Ser
caagtgaagccttccgagcc

SEQ ID NO: 518 - DNA, 20 bp, Artificial Sequence
  Asn544Ser
ggctcggaaggcttcacttg

SEQ ID NO: 519 - DNA, 25 bp, Artificial Sequence
  Asn544Ser
caccgggctcggaaggcttcacttg

SEQ ID NO: 520 - DNA, 25 bp, Artificial Sequence
  Asn544Ser
caaacaagtgaagccttccgagccc

SEQ ID NO: 521 - DNA, 20 bp, Artificial Sequence
  Ala546Thr
caaatgaaaccttccgagcc

SEQ ID NO: 522 - DNA, 20 bp, Artificial Sequence
  Ala546Thr
ggctcggaaggtttcatttg

SEQ ID NO: 523 - DNA, 25 bp, Artificial Sequence
  Ala546Thr
caccgggctcggaaggtttcatttg

SEQ ID NO: 524 - DNA, 25 bp, Artificial Sequence
  Ala546Thr
caaacaaatgaaaccttccgagccc

SEQ ID NO: 525 - DNA, 20 bp, Artificial Sequence
  Ala546Asp
caaatgaagacttccgagcc

SEQ ID NO: 526 - DNA, 20 bp, Artificial Sequence
  Ala546Asp
ggctcggaagtcttcatttg

SEQ ID NO: 527 - DNA, 25 bp, Artificial Sequence
  Ala546Asp
caccgggctcggaagtcttcatttg

SEQ ID NO: 528 - DNA, 25 bp, Artificial Sequence
  Ala546Asp
caaacaaatgaagacttccgagccc

SEQ ID NO: 529 - DNA, 20 bp, Artificial Sequence
  Phe547Ser
gagccctgccaccaagagaa

SEQ ID NO: 530 - DNA, 20 bp, Artificial Sequence
  Phe547Ser
ttctcttggtggcagggctc

SEQ ID NO: 531 - DNA, 25 bp, Artificial Sequence
  Phe547Ser
caccgttctcttggtggcagggctc

SEQ ID NO: 532 - DNA, 25 bp, Artificial Sequence
  Phe547Ser
caaagagccctgccaccaagagaac

SEQ ID NO: 533 - DNA, 20 bp, Artificial Sequence
  Phe547Ser
cgagccctgccaccaagaga

SEQ ID NO: 534 - DNA, 20 bp, Artificial Sequence
  Phe547Ser
tctcttggtggcagggctcg

SEQ ID NO: 535 - DNA, 25 bp, Artificial Sequence
  Phe547Ser
caccgtctcttggtggcagggctcg

SEQ ID NO: 536 - DNA, 25 bp, Artificial Sequence
  Phe547Ser
caaacgagccctgccaccaagagac

SEQ ID NO: 537 - DNA, 20 bp, Artificial Sequence
  Pro551Gln
gcaaccaagagaacggagca

SEQ ID NO: 538 - DNA, 20 bp, Artificial Sequence
  Pro551Gln
tgctccgttctcttggttgc

SEQ ID NO: 539 - DNA, 25 bp, Artificial Sequence
  Pro551Gln
caccgtgctccgttctcttggttgc

SEQ ID NO: 540 - DNA, 25 bp, Artificial Sequence
  Pro551Gln
caaagcaaccaagagaacggagcac

SEQ ID NO: 541 - DNA, 20 bp, Artificial Sequence
  Leu558Pro
ttgggtaaagaccaacttaa

SEQ ID NO: 542 - DNA, 20 bp, Artificial Sequence
  Leu558Pro
ttaagttggtctttacccaa

SEQ ID NO: 543 - DNA, 25 bp, Artificial Sequence
  Leu558Pro
caccgttaagttggtctttacccaa

SEQ ID NO: 544 - DNA, 25 bp, Artificial Sequence
  Leu558Pro
caaattgggtaaagaccaacttaac

SEQ ID NO: 545 - DNA, 21 bp, Artificial Sequence
  Leu558Pro
tacttaagttggtctttaccc

SEQ ID NO: 546 - DNA, 21 bp, Artificial Sequence
  Leu558Pro
gggtaaagaccaacttaagta

SEQ ID NO: 547 - DNA, 26 bp, Artificial Sequence
  Leu558Pro
caccggggtaaagaccaacttaagta

SEQ ID NO: 548 - DNA, 26 bp, Artificial Sequence
  Leu558Pro
caaatacttaagttggtctttacccc

SEQ ID NO: 549 - DNA, 20 bp, Artificial Sequence
  Leu558Pro
aagagaacggagcagaccct

SEQ ID NO: 550 - DNA, 20 bp, Artificial Sequence
  Leu558Pro
aagagaacggagcagaccct

SEQ ID NO: 551 - DNA, 25 bp, Artificial Sequence
  Leu558Pro
caccgaagagaacggagcagaccct

SEQ ID NO: 552 - DNA, 25 bp, Artificial Sequence
  Leu558Pro
caaaagggtctgctccgttctcttc

SEQ ID NO: 553 - DNA, 21 bp, Artificial Sequence
  Leu558Pro
ccaagagaacggagcagaccc

SEQ ID NO: 554 - DNA, 21 bp, Artificial Sequence
  Leu558Pro
ccaagagaacggagcagaccc

SEQ ID NO: 555 - DNA, 26 bp, Artificial Sequence
  Leu558Pro
caccgccaagagaacggagcagaccc

SEQ ID NO: 556 - DNA, 26 bp, Artificial Sequence
  Leu558Pro
caaagggtctgctccgttctcttggc

SEQ ID NO: 557 - DNA, 20 bp, Artificial Sequence
  His572del
gccaacatcctgaaatacat

SEQ ID NO: 558 - DNA, 20 bp, Artificial Sequence
  His572del
gccaacatcctgaaatacat

SEQ ID NO: 559 - DNA, 25 bp, Artificial Sequence
  His572del
caccggccaacatcctgaaatacat

SEQ ID NO: 560 - DNA, 25 bp, Artificial Sequence
  His572del
caaaatgtatttcaggatgttggcc

SEQ ID NO: 561 - DNA, 19 bp, Artificial Sequence
  His572del
aaatacattggtgatgaaa

SEQ ID NO: 562 - DNA, 19 bp, Artificial Sequence
  His572del
tttcatcaccaatgtattt

SEQ ID NO: 563 - DNA, 24 bp, Artificial Sequence
  His572del
caccgtttcatcaccaatgtattt

SEQ ID NO: 564 - DNA, 24 bp, Artificial Sequence
  His572del
caaaaaatacattggtgatgaaac

SEQ ID NO: 565 - DNA, 20 bp, Artificial Sequence
  Gly594Val
agttgacaagctggaagtca

SEQ ID NO: 566 - DNA, 20 bp, Artificial Sequence
  Gly594Val
tgacttccagcttgtcaact

SEQ ID NO: 567 - DNA, 25 bp, Artificial Sequence
  Gly594Val
caccgtgacttccagcttgtcaact

SEQ ID NO: 568 - DNA, 25 bp, Artificial Sequence
  Gly594Val
caaaagttgacaagctggaagtcac

SEQ ID NO: 569 - DNA, 20 bp, Artificial Sequence
  Val613del
gacatcatggccacaaaatg

SEQ ID NO: 570 - DNA, 20 bp, Artificial Sequence
  Val613del
cattttgtggccatgatgtc

SEQ ID NO: 571 - DNA, 25 bp, Artificial Sequence
  Val613del
caccgcattttgtggccatgatgtc

SEQ ID NO: 572 - DNA, 25 bp, Artificial Sequence
  Val613del
caaagacatcatggccacaaaatgc

SEQ ID NO: 573 - DNA, 20 bp, Artificial Sequence
  Val613Gly
ggtgccgagcctgacatcat

SEQ ID NO: 574 - DNA, 20 bp, Artificial Sequence
  Val613Gly
atgatgtcaggctcggcacc

SEQ ID NO: 575 - DNA, 25 bp, Artificial Sequence
  Val613Gly
caccgatgatgtcaggctcggcacc

SEQ ID NO: 576 - DNA, 25 bp, Artificial Sequence
  Val613Gly
caaaggtgccgagcctgacatcatc

SEQ ID NO: 577 - DNA, 20 bp, Artificial Sequence
  Val613Gly
gtgagtgtcaacaaggagcc

SEQ ID NO: 578 - DNA, 20 bp, Artificial Sequence
  Val613Gly
gtgagtgtcaacaaggagcc

SEQ ID NO: 579 - DNA, 25 bp, Artificial Sequence
  Val613Gly
caccggtgagtgtcaacaaggagcc

SEQ ID NO: 580 - DNA, 25 bp, Artificial Sequence
  Val613Gly
caaaggctccttgttgacactcacc

SEQ ID NO: 581 - DNA, 20 bp, Artificial Sequence
  Met619Lys
gacatcaaggccacaaatgg

SEQ ID NO: 582 - DNA, 20 bp, Artificial Sequence
  Met619Lys
ccatttgtggccttgatgtc

SEQ ID NO: 583 - DNA, 25 bp, Artificial Sequence
  Met619Lys
caccgccatttgtggccttgatgtc

SEQ ID NO: 584 - DNA, 25 bp, Artificial Sequence
  Met619Lys
caaagacatcaaggccacaaatggc

SEQ ID NO: 585 - DNA, 20 bp, Artificial Sequence
  Ala620Asp
cctgacatcatggacacaaa

SEQ ID NO: 586 - DNA, 20 bp, Artificial Sequence
  Ala620Asp
cctgacatcatggacacaaa

SEQ ID NO: 587 - DNA, 25 bp, Artificial Sequence
  Ala620Asp
caccgcctgacatcatggacacaaa

SEQ ID NO: 588 - DNA, 25 bp, Artificial Sequence
  Ala620Asp
caaatttgtgtccatgatgtcaggc

SEQ ID NO: 589 - DNA, 20 bp, Artificial Sequence
  Asn622His
cctgacatcatggccacaca

SEQ ID NO: 590 - DNA, 20 bp, Artificial Sequence
  Asn622His
cctgacatcatggccacaca

SEQ ID NO: 591 - DNA, 25 bp, Artificial Sequence
  Asn622His
caccgcctgacatcatggccacaca

SEQ ID NO: 592 - DNA, 25 bp, Artificial Sequence
  Asn622His
caaatgtgtggccatgatgtcaggc

SEQ ID NO: 593 - DNA, 20 bp, Artificial Sequence
  Asn622Lys
catcatggccacaaaaggcg

SEQ ID NO: 594 - DNA, 20 bp, Artificial Sequence
  Asn622Lys
catcatggccacaaaaggcg

SEQ ID NO: 595 - DNA, 25 bp, Artificial Sequence
  Asn622Lys
caccgcatcatggccacaaaaggcg

SEQ ID NO: 596 - DNA, 25 bp, Artificial Sequence
  Asn622Lys
caaacgccttttgtggccatgatgc

SEQ ID NO: 597 - DNA, 20 bp, Artificial Sequence
  Asn622Lys
ccctgacatcatggccacaa

SEQ ID NO: 598 - DNA, 20 bp, Artificial Sequence
  Asn622Lys
ccctgacatcatggccacaa

SEQ ID NO: 599 - DNA, 25 bp, Artificial Sequence
  Asn622Lys
caccgccctgacatcatggccacaa

SEQ ID NO: 600 - DNA, 25 bp, Artificial Sequence
  Asn622Lys
caaattgtggccatgatgtcagggc

SEQ ID NO: 601 - DNA, 20 bp, Artificial Sequence
  Asn622Lys
catcatggccacaaagggcg

SEQ ID NO: 602 - DNA, 20 bp, Artificial Sequence
  Asn622Lys
catcatggccacaaagggcg

SEQ ID NO: 603 - DNA, 25 bp, Artificial Sequence
  Asn622Lys
caccgcatcatggccacaaagggcg

SEQ ID NO: 604 - DNA, 25 bp, Artificial Sequence
  Asn622Lys
caaacgccctttgtggccatgatgc

SEQ ID NO: 605 - DNA, 20 bp, Artificial Sequence
  Gly623Arg
catcatggccacaaatcgcg

SEQ ID NO: 606 - DNA, 20 bp, Artificial Sequence
  Gly623Arg
catcatggccacaaatcgcg

SEQ ID NO: 607 - DNA, 25 bp, Artificial Sequence
  Gly623Arg
caccgcatcatggccacaaatcgcg

SEQ ID NO: 608 - DNA, 25 bp, Artificial Sequence
  Gly623Arg
caaacgcgatttgtggccatgatgc

SEQ ID NO: 609 - DNA, 20 bp, Artificial Sequence
  Gly623Asp
catcatggccacaaatgacg

SEQ ID NO: 610 - DNA, 20 bp, Artificial Sequence
  Gly623Asp
catcatggccacaaatgacg

SEQ ID NO: 611 - DNA, 25 bp, Artificial Sequence
  Gly623Asp
caccgcatcatggccacaaatgacg

SEQ ID NO: 612 - DNA, 25 bp, Artificial Sequence
  Gly623Asp
caaacgtcatttgtggccatgatgc

SEQ ID NO: 613 - DNA, 20 bp, Artificial Sequence
  Val624_Val625del
caaatggccatgtcatcacc

SEQ ID NO: 614 - DNA, 20 bp, Artificial Sequence
  Val624_Val625del
ggtgatgacatggccatttg

SEQ ID NO: 615 - DNA, 25 bp, Artificial Sequence
  Val624_Val625del
caccgggtgatgacatggccatttg

SEQ ID NO: 616 - DNA, 25 bp, Artificial Sequence
  Val624_Val625del
caaacaaatggccatgtcatcaccc

SEQ ID NO: 617 - DNA, 20 bp, Artificial Sequence
  Val624Met
catcatggccacaaatggca

SEQ ID NO: 618 - DNA, 20 bp, Artificial Sequence
  Val624Met
catcatggccacaaatggca

SEQ ID NO: 619 - DNA, 25 bp, Artificial Sequence
  Val624Met
caccgcatcatggccacaaatggca

SEQ ID NO: 620 - DNA, 25 bp, Artificial Sequence
  Val624Met
caaatgccatttgtggccatgatgc

SEQ ID NO: 621 - DNA, 20 bp, Artificial Sequence
  Val625Asp
caaatggcgtgatccatgtc

SEQ ID NO: 622 - DNA, 20 bp, Artificial Sequence
  Val625Asp
gacatggatcacgccatttg

SEQ ID NO: 623 - DNA, 25 bp, Artificial Sequence
  Val625Asp
caccggacatggatcacgccatttg

SEQ ID NO: 624 - DNA, 25 bp, Artificial Sequence
  Val625Asp
caaacaaatggcgtgatccatgtcc

SEQ ID NO: 625 - DNA, 20 bp, Artificial Sequence
  His626Arg
caaatggcgtggtccgtgtc

SEQ ID NO: 626 - DNA, 20 bp, Artificial Sequence
  His626Arg
gacacggaccacgccatttg

SEQ ID NO: 627 - DNA, 25 bp, Artificial Sequence
  His626Arg
caccggacacggaccacgccatttg

SEQ ID NO: 628 - DNA, 25 bp, Artificial Sequence
  His626Arg
caaacaaatggcgtggtccgtgtcc

SEQ ID NO: 629 - DNA, 20 bp, Artificial Sequence
  His626Pro
gtcatcaccaatgttctgca

SEQ ID NO: 630 - DNA, 20 bp, Artificial Sequence
  His626Pro
tgcagaacattggtgatgac

SEQ ID NO: 631 - DNA, 25 bp, Artificial Sequence
  His626Pro
caccgtgcagaacattggtgatgac

SEQ ID NO: 632 - DNA, 25 bp, Artificial Sequence
  His626Pro
caaagtcatcaccaatgttctgcac

SEQ ID NO: 633 - DNA, 20 bp, Artificial Sequence
  His626Pro
caaatggcgtggtccctgtc

SEQ ID NO: 634 - DNA, 20 bp, Artificial Sequence
  His626Pro
gacagggaccacgccatttg

SEQ ID NO: 635 - DNA, 25 bp, Artificial Sequence
  His626Pro
caccggacagggaccacgccatttg

SEQ ID NO: 636 - DNA, 25 bp, Artificial Sequence
  His626Pro
caaacaaatggcgtggtccctgtcc

SEQ ID NO: 637 - DNA, 20 bp, Artificial Sequence
  Val627SerfsX44
caccaatgttctgcagcctc

SEQ ID NO: 638 - DNA, 20 bp, Artificial Sequence
  Val627SerfsX44
gaggctgcagaacattggtg

SEQ ID NO: 639 - DNA, 25 bp, Artificial Sequence
  Val627SerfsX44
caccggaggctgcagaacattggtg

SEQ ID NO: 640 - DNA, 25 bp, Artificial Sequence
  Val627SerfsX44
caaacaccaatgttctgcagcctcc

SEQ ID NO: 641 - DNA, 20 bp, Artificial Sequence
  Val627SerfsX44
ttcatcaccaatgttctgca

SEQ ID NO: 642 - DNA, 20 bp, Artificial Sequence
  Val627SerfsX44
tgcagaacattggtgatgaa

SEQ ID NO: 643 - DNA, 25 bp, Artificial Sequence
  Val627SerfsX44
caccgtgcagaacattggtgatgaa

SEQ ID NO: 644 - DNA, 25 bp, Artificial Sequence
  Val627SerfsX44
caaattcatcaccaatgttctgcac

SEQ ID NO: 645 - DNA, 13 bp, Artificial Sequence
  Thr629_Asn630insAsnValPro
tgcagcctccagg

SEQ ID NO: 646 - DNA, 20 bp, Artificial Sequence
  Thr629_Asn630insAsnValPro
cctggaggctgcagaacatt

SEQ ID NO: 647 - DNA, 25 bp, Artificial Sequence
  Thr629_Asn630insAsnValPro
caccgcctggaggctgcagaacatt

SEQ ID NO: 648 - DNA, 18 bp, Artificial Sequence
  Thr629_Asn630insAsnValPro
caaatgcagcctccaggc

SEQ ID NO: 649 - DNA, 20 bp, Artificial Sequence
  Val631Asp
atgatctgcagcctccaggt

SEQ ID NO: 650 - DNA, 20 bp, Artificial Sequence
  Val631Asp
acctggaggctgcagatcat

SEQ ID NO: 651 - DNA, 25 bp, Artificial Sequence
  Val631Asp
caccgacctggaggctgcagatcat

SEQ ID NO: 652 - DNA, 25 bp, Artificial Sequence
  Val631Asp
caaaatgatctgcagcctccaggtc

SEQ ID NO: 653 - DNA, 20 bp, Artificial Sequence
  Arg666Ser
gagctctgtgcgactagccc

SEQ ID NO: 654 - DNA, 20 bp, Artificial Sequence
  Arg666Ser
gggctagtcgcacagagctc

SEQ ID NO: 655 - DNA, 25 bp, Artificial Sequence
  Arg666Ser
caccggggctagtcgcacagagctc

SEQ ID NO: 656 - DNA, 25 bp, Artificial Sequence
  Arg666Ser
caaagagctctgtgcgactagcccc

SEQ ID NO: 657 - DNA, 19 bp, Artificial Sequence
  Arg555Trp
ttccgagccctgccaccaa

SEQ ID NO: 658 - DNA, 19 bp, Artificial Sequence
  Arg555Trp
ttccgagccctgccaccaa

SEQ ID NO: 659 - DNA, 24 bp, Artificial Sequence
  Arg555Trp
caccgttccgagccctgccaccaa

SEQ ID NO: 660 - DNA, 24 bp, Artificial Sequence
  Arg555Trp
caaattggtggcagggctcggaac

SEQ ID NO: 661 - DNA, 20 bp, Artificial Sequence
  Arg555Trp
agagaatggagcagactctt

SEQ ID NO: 662 - DNA, 20 bp, Artificial Sequence
  Arg555Trp
aagagtctgctccattctct

SEQ ID NO: 663 - DNA, 25 bp, Artificial Sequence
  Arg555Trp
caccgaagagtctgctccattctct

SEQ ID NO: 664 - DNA, 25 bp, Artificial Sequence
  Arg555Trp
caaaagagaatggagcagactcttc

SEQ ID NO: 665 - DNA, 20 bp, Artificial Sequence
  Arg124Ser
tcagctgtacacggacagca

SEQ ID NO: 666 - DNA, 20 bp, Artificial Sequence
  Arg124Ser
tcagctgtacacggacagca

SEQ ID NO: 667 - DNA, 25 bp, Artificial Sequence
  Arg124Ser
caccgtcagctgtacacggacagca

SEQ ID NO: 668 - DNA, 25 bp, Artificial Sequence
  Arg124Ser
caaatgctgtccgtgtacagctgac

SEQ ID NO: 669 - DNA, 20 bp, Artificial Sequence
  Asp123delins
tgtacacggacctcaagctg

SEQ ID NO: 670 - DNA, 20 bp, Artificial Sequence
  Asp123delins
tgtacacggacctcaagctg

SEQ ID NO: 671 - DNA, 25 bp, Artificial Sequence
  Asp123delins
caccgtgtacacggacctcaagctg

SEQ ID NO: 672 - DNA, 25 bp, Artificial Sequence
  Asp123delins
caaacagcttgaggtccgtgtacac

SEQ ID NO: 673 - DNA, 21 bp, Artificial Sequence
  Asp123delins
ctgtacacggacctcaagctg

SEQ ID NO: 674 - DNA, 21 bp, Artificial Sequence
  Asp123delins
cagcttgaggtccgtgtacag

SEQ ID NO: 675 - DNA, 26 bp, Artificial Sequence
  Asp123delins
caccgcagcttgaggtccgtgtacag

SEQ ID NO: 676 - DNA, 26 bp, Artificial Sequence
  Asp123delins
caaactgtacacggacctcaagctgc

SEQ ID NO: 677 - DNA, 20 bp, Artificial Sequence
  Arg124His
tcagctgtacacggaccaca

SEQ ID NO: 678 - DNA, 20 bp, Artificial Sequence
  Arg124His
tcagctgtacacggaccaca

SEQ ID NO: 679 - DNA, 25 bp, Artificial Sequence
  Arg124His
caccgtcagctgtacacggaccaca

SEQ ID NO: 680 - DNA, 25 bp, Artificial Sequence
  Arg124His
caaatgtggtccgtgtacagctgac

SEQ ID NO: 681 - DNA, 22 bp, Artificial Sequence
  Arg124His
ctgtacacggaccacacggaga

SEQ ID NO: 682 - DNA, 22 bp, Artificial Sequence
  Arg124His
tctccgtgtggtccgtgtacag

SEQ ID NO: 683 - DNA, 27 bp, Artificial Sequence
  Arg124His
caccgtctccgtgtggtccgtgtacag

SEQ ID NO: 684 - DNA, 27 bp, Artificial Sequence
  Arg124His
caaactgtacacggaccacacggagac

SEQ ID NO: 685 - DNA, 20 bp, Artificial Sequence
  Arg124Leu
tcagctgtacacggacctca

SEQ ID NO: 686 - DNA, 20 bp, Artificial Sequence
  Arg124Leu
tcagctgtacacggacctca

SEQ ID NO: 687 - DNA, 25 bp, Artificial Sequence
  Arg124Leu
caccgtcagctgtacacggacctca

SEQ ID NO: 688 - DNA, 25 bp, Artificial Sequence
  Arg124Leu
caaatgaggtccgtgtacagctgac

SEQ ID NO: 689 - DNA, 20 bp, Artificial Sequence
  Arg124Leu
aagggagacaatcgctttag

SEQ ID NO: 690 - DNA, 22 bp, Artificial Sequence
  Arg124Leu
tctccgtgaggtccgtgtacag

SEQ ID NO: 691 - DNA, 27 bp, Artificial Sequence
  Arg124Leu
caccgtctccgtgaggtccgtgtacag

SEQ ID NO: 692 - DNA, 25 bp, Artificial Sequence
  Arg124Leu
caaaaagggagacaatcgctttagc

SEQ ID NO: 693 - DNA, 20 bp, Artificial Sequence
  Leu509Pro
aagggagacaatcgctttag

SEQ ID NO: 694 - DNA, 20 bp, Artificial Sequence
  Leu509Pro
ctaaagcgattgtctccctt

SEQ ID NO: 695 - DNA, 25 bp, Artificial Sequence
  Leu509Pro
caccgctaaagcgattgtctccctt

SEQ ID NO: 696 - DNA, 25 bp, Artificial Sequence
  Leu509Pro
caaaaagggagacaatcgctttagc

SEQ ID NO: 697 - DNA, 20 bp, Artificial Sequence
  Leu509Pro
gactgtcatggatgtcccga

SEQ ID NO: 698 - DNA, 20 bp, Artificial Sequence
  Leu509Pro
gactgtcatggatgtcccga

SEQ ID NO: 699 - DNA, 25 bp, Artificial Sequence
  Leu509Pro
caccggactgtcatggatgtcccga

SEQ ID NO: 700 - DNA, 25 bp, Artificial Sequence
  Leu509Pro
caaatcgggacatccatgacagtcc

SEQ ID NO: 701 - DNA, 20 bp, Artificial Sequence
  Leu103_Ser104del
acctttacgagaccctggga

SEQ ID NO: 702 - DNA, 20 bp, Artificial Sequence
  Leu103_Ser104del
tcccagggtctcgtaaaggt

SEQ ID NO: 703 - DNA, 25 bp, Artificial Sequence
  Leu103_Ser104del
caccgtcccagggtctcgtaaaggt

SEQ ID NO: 704 - DNA, 25 bp, Artificial Sequence
  Leu103_Ser104del
caaaacctttacgagaccctgggac

SEQ ID NO: 705 - DNA, 20 bp, Artificial Sequence
  Leu103_Ser104del
ctcaaacctttacgagaccc

SEQ ID NO: 706 - DNA, 20 bp, Artificial Sequence
  Leu103_Ser104del
gggtctcgtaaaggtttgag

SEQ ID NO: 707 - DNA, 25 bp, Artificial Sequence
  Leu103_Ser104del
caccggggtctcgtaaaggtttgag

SEQ ID NO: 708 - DNA, 25 bp, Artificial Sequence
  Leu103_Ser104del
caaactcaaacctttacgagacccc

SEQ ID NO: 709 - DNA, 20 bp, Artificial Sequence
  Val113Ile
tacgagaccctgggagtcat

SEQ ID NO: 710 - DNA, 20 bp, Artificial Sequence
  Val113Ile
tacgagaccctgggagtcat

SEQ ID NO: 711 - DNA, 25 bp, Artificial Sequence
  Val113Ile
caccgtacgagaccctgggagtcat

SEQ ID NO: 712 - DNA, 25 bp, Artificial Sequence
  Val113Ile
caaaatgactcccagggtctcgtac

SEQ ID NO: 713 - DNA, 20 bp, Artificial Sequence
  Val113Ile
ttacgagaccctgggagtca

SEQ ID NO: 714 - DNA, 20 bp, Artificial Sequence
  Val113Ile
ttacgagaccctgggagtca

SEQ ID NO: 715 - DNA, 25 bp, Artificial Sequence
  Val113Ile
caccgttacgagaccctgggagtca

SEQ ID NO: 716 - DNA, 25 bp, Artificial Sequence
  Val113Ile
caaatgactcccagggtctcgtaac

SEQ ID NO: 717 - DNA, 20 bp, Artificial Sequence
  Asp123His
tcagctgtacacgcaccgca

SEQ ID NO: 718 - DNA, 20 bp, Artificial Sequence
  Asp123His
tcagctgtacacgcaccgca

SEQ ID NO: 719 - DNA, 25 bp, Artificial Sequence
  Asp123His
caccgtcagctgtacacgcaccgca

SEQ ID NO: 720 - DNA, 25 bp, Artificial Sequence
  Asp123His
caaatgcggtgcgtgtacagctgac

SEQ ID NO: 721 - DNA, 21 bp, Artificial Sequence
  Asp123His
ctgtacacgcaccgcacggag

SEQ ID NO: 722 - DNA, 21 bp, Artificial Sequence
  Asp123His
ctccgtgcggtgcgtgtacag

SEQ ID NO: 723 - DNA, 26 bp, Artificial Sequence
  Asp123His
caccgctccgtgcggtgcgtgtacag

SEQ ID NO: 724 - DNA, 26 bp, Artificial Sequence
  Asp123His
caaactgtacacgcaccgcacggagc

SEQ ID NO: 725 - DNA, 20 bp, Artificial Sequence
  Arg124Leu
tcagctgtacacggacctca

SEQ ID NO: 726 - DNA, 20 bp, Artificial Sequence
  Arg124Leu
tcagctgtacacggacctca

SEQ ID NO: 727 - DNA, 25 bp, Artificial Sequence
  Arg124Leu
caccgtcagctgtacacggacctca

SEQ ID NO: 728 - DNA, 25 bp, Artificial Sequence
  Arg124Leu
caaatgaggtccgtgtacagctgac

SEQ ID NO: 729 - DNA, 20 bp, Artificial Sequence
  Thr125_Glu126del
caagctgaggcctgagatgg

SEQ ID NO: 730 - DNA, 20 bp, Artificial Sequence
  Thr125_Glu126del
ccatctcaggcctcagcttg

SEQ ID NO: 731 - DNA, 25 bp, Artificial Sequence
  Thr125_Glu126del
caccgccatctcaggcctcagcttg

SEQ ID NO: 732 - DNA, 25 bp, Artificial Sequence
  Thr125_Glu126del
caaacaagctgaggcctgagatggc

SEQ ID NO: 733 - DNA, 21 bp, Artificial Sequence
  Thr125_Glu126del
ctgtacacggaccgcaagctg

SEQ ID NO: 734 - DNA, 21 bp, Artificial Sequence
  Thr125_Glu126del
cagcttgcggtccgtgtacag

SEQ ID NO: 735 - DNA, 26 bp, Artificial Sequence
  Thr125_Glu126del
caccgcagcttgcggtccgtgtacag

SEQ ID NO: 736 - DNA, 26 bp, Artificial Sequence
  Thr125_Glu126del
caaactgtacacggaccgcaagctgc

SEQ ID NO: 737 - DNA, 20 bp, Artificial Sequence
  Ala97Thr
gtcctggctgtgcacgggac

SEQ ID NO: 738 - DNA, 20 bp, Artificial Sequence
  Ala97Thr
gtcctggctgtgcacgggac

SEQ ID NO: 739 - DNA, 25 bp, Artificial Sequence
  Ala97Thr
caccggtcctggctgtgcacgggac

SEQ ID NO: 740 - DNA, 25 bp, Artificial Sequence
  Ala97Thr
caaagtcccgtgcacagccaggacc

SEQ ID NO: 741 - DNA, 20 bp, Artificial Sequence
  Gly98Ser
tgtgcacggggccagtaatt

SEQ ID NO: 742 - DNA, 20 bp, Artificial Sequence
  Gly98Ser
tgtgcacggggccagtaatt

SEQ ID NO: 743 - DNA, 25 bp, Artificial Sequence
  Gly98Ser
caccgtgtgcacggggccagtaatt

SEQ ID NO: 744 - DNA, 25 bp, Artificial Sequence
  Gly98Ser
caaaaattactggccccgtgcacac

SEQ ID NO: 745 - DNA, 20 bp, Artificial Sequence
  Asn102Ser
gtaatttggtcagcacttac

SEQ ID NO: 746 - DNA, 20 bp, Artificial Sequence
  Asn102Ser
gtaagtgctgaccaaattac

SEQ ID NO: 747 - DNA, 25 bp, Artificial Sequence
  Asn102Ser
caccggtaagtgctgaccaaattac

SEQ ID NO: 748 - DNA, 25 bp, Artificial Sequence
  Asn102Ser
caaagtaatttggtcagcacttacc

SEQ ID NO: 749 - DNA, 21 bp, Artificial Sequence
  Asp112Asn
tccaagggcattaaccacaaa

SEQ ID NO: 750 - DNA, 21 bp, Artificial Sequence
  Asp112Asn
tccaagggcattaaccacaaa

SEQ ID NO: 751 - DNA, 26 bp, Artificial Sequence
  Asp112Asn
caccgtccaagggcattaaccacaaa

SEQ ID NO: 752 - DNA, 26 bp, Artificial Sequence
  Asp112Asn
caaatttgtggttaatgcccttggac

SEQ ID NO: 753 - DNA, 20 bp, Artificial Sequence
  Asp112Asn
agggcattaaccacaaaaag

SEQ ID NO: 754 - DNA, 20 bp, Artificial Sequence
  Asp112Asn
ctttttgtggttaatgccct

SEQ ID NO: 755 - DNA, 25 bp, Artificial Sequence
  Asp112Asn
caccgctttttgtggttaatgccct

SEQ ID NO: 756 - DNA, 25 bp, Artificial Sequence
  Asp112Asn
caaaagggcattaaccacaaaaagc

SEQ ID NO: 757 - DNA, 20 bp, Artificial Sequence
  Asp112Gly
tatgacttttccaagggcat

SEQ ID NO: 758 - DNA, 20 bp, Artificial Sequence
  Asp112Gly
tatgacttttccaagggcat

SEQ ID NO: 759 - DNA, 25 bp, Artificial Sequence
  Asp112Gly
caccgtatgacttttccaagggcat

SEQ ID NO: 760 - DNA, 25 bp, Artificial Sequence
  Asp112Gly
caaaatgcccttggaaaagtcatac

SEQ ID NO: 761 - DNA, 20 bp, Artificial Sequence
  Asp112Gly
agggcattggccacaaaaag

SEQ ID NO: 762 - DNA, 20 bp, Artificial Sequence
  Asp112Gly
ctttttgtggccaatgccct

SEQ ID NO: 763 - DNA, 25 bp, Artificial Sequence
  Asp112Gly
caccgctttttgtggccaatgccct

SEQ ID NO: 764 - DNA, 25 bp, Artificial Sequence
  Asp112Gly
caaaagggcattggccacaaaaagc

SEQ ID NO: 765 - DNA, 21 bp, Artificial Sequence
  Asp112Gly
tccaagggcattggccacaaa

SEQ ID NO: 766 - DNA, 21 bp, Artificial Sequence
  Asp112Gly
tccaagggcattggccacaaa

SEQ ID NO: 767 - DNA, 26 bp, Artificial Sequence
  Asp112Gly
caccgtccaagggcattggccacaaa

SEQ ID NO: 768 - DNA, 26 bp, Artificial Sequence
  Asp112Gly
caaatttgtggccaatgcccttggac

SEQ ID NO: 769 - DNA, 20 bp, Artificial Sequence
  Asp118Gly
attgaccacaaaaagagtga

SEQ ID NO: 770 - DNA, 20 bp, Artificial Sequence
  Asp118Gly
attgaccacaaaaagagtga

SEQ ID NO: 771 - DNA, 25 bp, Artificial Sequence
  Asp118Gly
caccgattgaccacaaaaagagtga

SEQ ID NO: 772 - DNA, 25 bp, Artificial Sequence
  Asp118Gly
caaatcactctttttgtggtcaatc

SEQ ID NO: 773 - DNA, 20 bp, Artificial Sequence
  Asp118Gly
gagtgatggcaggacacttg

SEQ ID NO: 774 - DNA, 20 bp, Artificial Sequence
  Asp118Gly
gagtgatggcaggacacttg

SEQ ID NO: 775 - DNA, 25 bp, Artificial Sequence
  Asp118Gly
caccggagtgatggcaggacacttg

SEQ ID NO: 776 - DNA, 25 bp, Artificial Sequence
  Asp118Gly
caaacaagtgtcctgccatcactcc

SEQ ID NO: 777 - DNA, 21 bp, Artificial Sequence
  Asp118Gly
tgatggcaggacacttgtgga

SEQ ID NO: 778 - DNA, 21 bp, Artificial Sequence
  Asp118Gly
tgatggcaggacacttgtgga

SEQ ID NO: 779 - DNA, 26 bp, Artificial Sequence
  Asp118Gly
caccgtgatggcaggacacttgtgga

SEQ ID NO: 780 - DNA, 26 bp, Artificial Sequence
  Asp118Gly
caaatccacaagtgtcctgccatcac

SEQ ID NO: 781 - DNA, 20 bp, Artificial Sequence
  Arg119Gly
gaccacaaaaagagtgatga

SEQ ID NO: 782 - DNA, 20 bp, Artificial Sequence
  Arg119Gly
gaccacaaaaagagtgatga

SEQ ID NO: 783 - DNA, 25 bp, Artificial Sequence
  Arg119Gly
caccggaccacaaaaagagtgatga

SEQ ID NO: 784 - DNA, 25 bp, Artificial Sequence
  Arg119Gly
caaatcatcactctttttgtggtcc

SEQ ID NO: 785 - DNA, 20 bp, Artificial Sequence
  Arg119Gly
gagtgatgacgggacacttg

SEQ ID NO: 786 - DNA, 20 bp, Artificial Sequence
  Arg119Gly
gagtgatgacgggacacttg

SEQ ID NO: 787 - DNA, 25 bp, Artificial Sequence
  Arg119Gly
caccggagtgatgacgggacacttg

SEQ ID NO: 788 - DNA, 25 bp, Artificial Sequence
  Arg119Gly
caaacaagtgtcccgtcatcactcc

SEQ ID NO: 789 - DNA, 20 bp, Artificial Sequence
  Arg119Gly
gatgacgggacacttgtgga

SEQ ID NO: 790 - DNA, 20 bp, Artificial Sequence
  Arg119Gly
gatgacgggacacttgtgga

SEQ ID NO: 791 - DNA, 25 bp, Artificial Sequence
  Arg119Gly
caccggatgacgggacacttgtgga

SEQ ID NO: 792 - DNA, 25 bp, Artificial Sequence
  Arg119Gly
caaatccacaagtgtcccgtcatcc

SEQ ID NO: 793 - DNA, 20 bp, Artificial Sequence
  Leu121Val
gagtgatgacaggacagttg

SEQ ID NO: 794 - DNA, 20 bp, Artificial Sequence
  Leu121Val
gagtgatgacaggacagttg

SEQ ID NO: 795 - DNA, 25 bp, Artificial Sequence
  Leu121Val
caccggagtgatgacaggacagttg

SEQ ID NO: 796 - DNA, 25 bp, Artificial Sequence
  Leu121Val
caaacaactgtcctgtcatcactcc

SEQ ID NO: 797 - DNA, 21 bp, Artificial Sequence
  Leu121Val
tgatgacaggacagttgtgga

SEQ ID NO: 798 - DNA, 21 bp, Artificial Sequence
  Leu121Val
tgatgacaggacagttgtgga

SEQ ID NO: 799 - DNA, 26 bp, Artificial Sequence
  Leu121Val
caccgtgatgacaggacagttgtgga

SEQ ID NO: 800 - DNA, 26 bp, Artificial Sequence
  Leu121Val
caaatccacaactgtcctgtcatcac

SEQ ID NO: 801 - DNA, 20 bp, Artificial Sequence
  Leu121Phe
gagtgatgacaggacatttg

SEQ ID NO: 802 - DNA, 20 bp, Artificial Sequence
  Leu121Phe
gagtgatgacaggacatttg

SEQ ID NO: 803 - DNA, 25 bp, Artificial Sequence
  Leu121Phe
caccggagtgatgacaggacatttg

SEQ ID NO: 804 - DNA, 25 bp, Artificial Sequence
  Leu121Phe
caaacaaatgtcctgtcatcactcc

SEQ ID NO: 805 - DNA, 21 bp, Artificial Sequence
  Leu121Phe
tgatgacaggacatttgtgga

SEQ ID NO: 806 - DNA, 21 bp, Artificial Sequence
  Leu121Phe
tgatgacaggacatttgtgga

SEQ ID NO: 807 - DNA, 26 bp, Artificial Sequence
  Leu121Phe
caccgtgatgacaggacatttgtgga

SEQ ID NO: 808 - DNA, 26 bp, Artificial Sequence
  Leu121Phe
caaatccacaaatgtcctgtcatcac

SEQ ID NO: 809 - DNA, 20 bp, Artificial Sequence
  Val122Glu
gatgacaggacacttgagga

SEQ ID NO: 810 - DNA, 20 bp, Artificial Sequence
  Val122Glu
gacacttgaggaccgaatct

SEQ ID NO: 811 - DNA, 25 bp, Artificial Sequence
  Val122Glu
caccggacacttgaggaccgaatct

SEQ ID NO: 812 - DNA, 25 bp, Artificial Sequence
  Val122Glu
caaaagattcggtcctcaagtgtcc

SEQ ID NO: 813 - DNA, 20 bp, Artificial Sequence
  Val122Glu
gatgacaggacacttgagga

SEQ ID NO: 814 - DNA, 20 bp, Artificial Sequence
  Val122Glu
gatgacaggacacttgagga

SEQ ID NO: 815 - DNA, 25 bp, Artificial Sequence
  Val122Glu
caccggatgacaggacacttgagga

SEQ ID NO: 816 - DNA, 25 bp, Artificial Sequence
  Val122Glu
caaatcctcaagtgtcctgtcatcc

SEQ ID NO: 817 - DNA, 20 bp, Artificial Sequence
  Val122Gly
aagagtgatgacaggacact

SEQ ID NO: 818 - DNA, 20 bp, Artificial Sequence
  Val122Gly
aagagtgatgacaggacact

SEQ ID NO: 819 - DNA, 25 bp, Artificial Sequence
  Val122Gly
caccgaagagtgatgacaggacact

SEQ ID NO: 820 - DNA, 25 bp, Artificial Sequence
  Val122Gly
caaaagtgtcctgtcatcactcttc

SEQ ID NO: 821 - DNA, 20 bp, Artificial Sequence
  Val122Gly
aagtgtcctgtcatcactct

SEQ ID NO: 822 - DNA, 20 bp, Artificial Sequence
  Val122Gly
agagtgatgacaggacactt

SEQ ID NO: 823 - DNA, 25 bp, Artificial Sequence
  Val122Gly
caccgagagtgatgacaggacactt

SEQ ID NO: 824 - DNA, 25 bp, Artificial Sequence
  Val122Gly
caaaaagtgtcctgtcatcactctc

SEQ ID NO: 825 - DNA, 20 bp, Artificial Sequence
  Val122Gly
gacacttggggaccgaatct

SEQ ID NO: 826 - DNA, 20 bp, Artificial Sequence
  Val122Gly
gacacttggggaccgaatct

SEQ ID NO: 827 - DNA, 25 bp, Artificial Sequence
  Val122Gly
caccggacacttggggaccgaatct

SEQ ID NO: 828 - DNA, 25 bp, Artificial Sequence
  Val122Gly
caaaagattcggtccccaagtgtcc

SEQ ID NO: 829 - DNA, 20 bp, Artificial Sequence
  Val122Gly
gatgacaggacacttgggga

SEQ ID NO: 830 - DNA, 20 bp, Artificial Sequence
  Val122Gly
gatgacaggacacttgggga

SEQ ID NO: 831 - DNA, 25 bp, Artificial Sequence
  Val122Gly
caccggatgacaggacacttgggga

SEQ ID NO: 832 - DNA, 25 bp, Artificial Sequence
  Val122Gly
caaatccccaagtgtcctgtcatcc

SEQ ID NO: 833 - DNA, 20 bp, Artificial Sequence
  Ser171Pro
tttctctacacaggaggtaa

SEQ ID NO: 834 - DNA, 20 bp, Artificial Sequence
  Ser171Pro
ttacctcctgtgtagagaaa

SEQ ID NO: 835 - DNA, 25 bp, Artificial Sequence
  Ser171Pro
caccgttacctcctgtgtagagaaa

SEQ ID NO: 836 - DNA, 25 bp, Artificial Sequence
  Ser171Pro
caaatttctctacacaggaggtaac

SEQ ID NO: 837 - DNA, 20 bp, Artificial Sequence
  Ser171Pro
gtctggcccctttctctaca

SEQ ID NO: 838 - DNA, 20 bp, Artificial Sequence
  Ser171Pro
tgtagagaaaggggccagac

SEQ ID NO: 839 - DNA, 25 bp, Artificial Sequence
  Ser171Pro
caccgtgtagagaaaggggccagac

SEQ ID NO: 840 - DNA, 25 bp, Artificial Sequence
  Ser171Pro
caaagtctggcccctttctctacac

SEQ ID NO: 841 - DNA, 20 bp, Artificial Sequence
  Tyr174Cys
ttctctgcacaggaggtaag

SEQ ID NO: 842 - DNA, 20 bp, Artificial Sequence
  Tyr174Cys
cttacctcctgtgcagagaa

SEQ ID NO: 843 - DNA, 25 bp, Artificial Sequence
  Tyr174Cys
caccgcttacctcctgtgcagagaa

SEQ ID NO: 844 - DNA, 25 bp, Artificial Sequence
  Tyr174Cys
caaattctctgcacaggaggtaagc

SEQ ID NO: 845 - DNA, 20 bp, Artificial Sequence
  Thr175Ile
tctggctcctttctctacat

SEQ ID NO: 846 - DNA, 20 bp, Artificial Sequence
  Thr175Ile
tctggctcctttctctacat

SEQ ID NO: 847 - DNA, 25 bp, Artificial Sequence
  Thr175Ile
caccgtctggctcctttctctacat

SEQ ID NO: 848 - DNA, 25 bp, Artificial Sequence
  Thr175Ile
caaaatgtagagaaaggagccagac

SEQ ID NO: 849 - DNA, 20 bp, Artificial Sequence
  Gly177Arg
tctacacaggacgtaagatt

SEQ ID NO: 850 - DNA, 20 bp, Artificial Sequence
  Gly177Arg
tctacacaggacgtaagatt

SEQ ID NO: 851 - DNA, 25 bp, Artificial Sequence
  Gly177Arg
caccgtctacacaggacgtaagatt

SEQ ID NO: 852 - DNA, 25 bp, Artificial Sequence
  Gly177Arg
caaaaatcttacgtcctgtgtagac

SEQ ID NO: 853 - DNA, 20 bp, Artificial Sequence
  Gly177Arg
tctacacaggaagtaagatt

SEQ ID NO: 854 - DNA, 20 bp, Artificial Sequence
  Gly177Arg
tctacacaggaagtaagatt

SEQ ID NO: 855 - DNA, 25 bp, Artificial Sequence
  Gly177Arg
caccgtctacacaggaagtaagatt

SEQ ID NO: 856 - DNA, 25 bp, Artificial Sequence
  Gly177Arg
caaaaatcttacttcctgtgtagac

SEQ ID NO: 857 - DNA, 20 bp, Artificial Sequence
  Lys181Arg
tggccgcaggaattggattc

SEQ ID NO: 858 - DNA, 20 bp, Artificial Sequence
  Lys181Arg
tggccgcaggaattggattc

SEQ ID NO: 859 - DNA, 25 bp, Artificial Sequence
  Lys181Arg
caccgtggccgcaggaattggattc

SEQ ID NO: 860 - DNA, 25 bp, Artificial Sequence
  Lys181Arg
caaagaatccaattcctgcggccac

SEQ ID NO: 861 - DNA, 20 bp, Artificial Sequence
  Lys181Arg
aggaattggattcaggtacg

SEQ ID NO: 862 - DNA, 20 bp, Artificial Sequence
  Lys181Arg
aggaattggattcaggtacg

SEQ ID NO: 863 - DNA, 25 bp, Artificial Sequence
  Lys181Arg
caccgaggaattggattcaggtacg

SEQ ID NO: 864 - DNA, 25 bp, Artificial Sequence
  Lys181Arg
caaacgtacctgaatccaattcctc

SEQ ID NO: 865 - DNA, 20 bp, Artificial Sequence
  Leu188His
cacatcatcctcatcacttt

SEQ ID NO: 866 - DNA, 20 bp, Artificial Sequence
  Leu188His
cacatcatcctcatcacttt

SEQ ID NO: 867 - DNA, 25 bp, Artificial Sequence
  Leu188His
caccgcacatcatcctcatcacttt

SEQ ID NO: 868 - DNA, 25 bp, Artificial Sequence
  Leu188His
caaaaaagtgatgaggatgatgtgc

SEQ ID NO: 869 - DNA, 20 bp, Artificial Sequence
  Asn232Ser
gcaacaccagggacatggag

SEQ ID NO: 870 - DNA, 20 bp, Artificial Sequence
  Asn232Ser
ctccatgtccctggtgttgc

SEQ ID NO: 871 - DNA, 25 bp, Artificial Sequence
  Asn232Ser
caccgctccatgtccctggtgttgc

SEQ ID NO: 872 - DNA, 25 bp, Artificial Sequence
  Asn232Ser
caaagcaacaccagggacatggagc

SEQ ID NO: 873 - DNA, 20 bp, Artificial Sequence
  Asn233His
caacaccagggacatggagt

SEQ ID NO: 874 - DNA, 20 bp, Artificial Sequence
  Asn233His
actccatgtccctggtgttg

SEQ ID NO: 875 - DNA, 25 bp, Artificial Sequence
  Asn233His
caccgactccatgtccctggtgttg

SEQ ID NO: 876 - DNA, 25 bp, Artificial Sequence
  Asn233His
caaacaacaccagggacatggagtc

SEQ ID NO: 877 - DNA, 20 bp, Artificial Sequence
  Asn233His
ttcccacaacaccagggaca

SEQ ID NO: 878 - DNA, 20 bp, Artificial Sequence
  Asn233His
tgtccctggtgttgtgggaa

SEQ ID NO: 879 - DNA, 25 bp, Artificial Sequence
  Asn233His
caccgtgtccctggtgttgtgggaa

SEQ ID NO: 880 - DNA, 25 bp, Artificial Sequence
  Asn233His
caaattcccacaacaccagggacac

SEQ ID NO: 881 - DNA, 21 bp, Artificial Sequence
  Asn233His
cattcccacaacaccagggac

SEQ ID NO: 882 - DNA, 21 bp, Artificial Sequence
  Asn233His
gtccctggtgttgtgggaatg

SEQ ID NO: 883 - DNA, 26 bp, Artificial Sequence
  Asn233His
caccggtccctggtgttgtgggaatg

SEQ ID NO: 884 - DNA, 26 bp, Artificial Sequence
  Asn233His
caaacattcccacaacaccagggacc

SEQ ID NO: 885 - DNA, 19 bp, Artificial Sequence
  Asp236Glu
tccaacaacaccagggaga

SEQ ID NO: 886 - DNA, 19 bp, Artificial Sequence
  Asp236Glu
tccaacaacaccagggaga

SEQ ID NO: 887 - DNA, 24 bp, Artificial Sequence
  Asp236Glu
caccgtccaacaacaccagggaga

SEQ ID NO: 888 - DNA, 24 bp, Artificial Sequence
  Asp236Glu
caaatctccctggtgttgttggac

SEQ ID NO: 889 - DNA, 19 bp, Artificial Sequence
  Asp236Glu
tccaacaacaccagggaga

SEQ ID NO: 890 - DNA, 19 bp, Artificial Sequence
  Asp236Glu
tccaacaacaccagggaga

SEQ ID NO: 891 - DNA, 24 bp, Artificial Sequence
  Asp236Glu
caccgtccaacaacaccagggaga

SEQ ID NO: 892 - DNA, 24 bp, Artificial Sequence
  Asp236Glu
caaatctccctggtgttgttggac

SEQ ID NO: 893 - DNA, 20 bp, Artificial Sequence
  Asp240Asn
ccagggacatggagtccaac

SEQ ID NO: 894 - DNA, 20 bp, Artificial Sequence
  Asp240Asn
ccagggacatggagtccaac

SEQ ID NO: 895 - DNA, 25 bp, Artificial Sequence
  Asp240Asn
caccgccagggacatggagtccaac

SEQ ID NO: 896 - DNA, 25 bp, Artificial Sequence
  Asp240Asn
caaagttggactccatgtccctggc

SEQ ID NO: 897 - DNA, 20 bp, artificial sequence
  guide sequence
gaactaattaccatgctaaa

SEQ ID NO: 898 - DNA, 20 bp, artificial sequence
  guide sequence
gagacaatcgctttagcatg

(19) United States
(12) Patent Application Publication
(10) Pub. No.: US 2019/0185850 A1
(43) Pub. Date: Jun. 20, 2019

(54) SINGLE GUIDE RNA/CRISPR/CAS9 SYSTEMS, AND METHODS OF USE THEREOF

(71) Applicant: Avellino Lab USA, Inc., Menlo Park, CA (US)

(72) Inventors: Tara Moore, Menlo Park, CA (US); Andrew Nesbit, Menlo Park, CA (US); David Courtney, Menlo Park, CA (US); Katie Christie, Menlo Park, CA (US); Gene Lee, Menlo Park, CA (US)

(73) Assignee: Avellino Lab USA, Inc., Menlo Park, CA (US)

(21) Appl. No.: 16/326,908

(22) PCT Filed: Aug. 21, 2017
(86) PCT No.: PCT/US2017/047861

§ 371 Date: Feb. 20, 2019

Related U.S. Application Data
(60) Provisional application No. 62/377,586, filed on Aug. 20, 2016.
(60) Provisional application No. 62/462,808, filed on Feb. 23, 2017.
(60) Provisional application No. 62/501,750, filed on May 5, 2017.

(51) Int. Cl. C12N 15/11; C12N 9/22; A61K 38/46; A61K 31/7088; A61K 9/00; A61P 27/02; C07K 14/315; C07K 14/195; C07K 14/495; A61K 35/545

(52) U.S. Cl. CPC C12N 15/11; C12N 9/22; A61K 38/465; A61K 31/7088; A61K 9/0048; A61P 27/02; C12N 2320/32; C07K 14/195; C07K 14/495; A61K 35/545; C12N 2310/20; C12N 2800/80; C07K 14/315

(57) ABSTRACT

The present disclosure relates to single guide RNA (sgRNA), Clustered Regularly Interspaced Short Palindromic Repeats (CRISPR)/CRISPR associate protein 9 (Cas9) system, and methods of use thereof for preventing, ameliorating or treating corneal dystrophies.

### FIELD OF THE INVENTION

The present disclosure relates to single guide RNA (sgRNA), Clustered Regularly Interspaced Short Palindromic Repeats (CRISPR)/CRISPR associate protein 9 (Cas9) system, and methods of use thereof for preventing, ameliorating or treating corneal dystrophies.

### BACKGROUND OF THE INVENTION

The majority of corneal dystrophies are inherited in an autosomal dominant fashion with a dominant-negative pathomechanism. For some genes, for example TGFBI and KRT12, it has been shown that they are haplosufficient; meaning one functional copy of the gene is sufficient to maintain normal function. By using siRNA that specifically targets the mutant allele, it is possible to overcome the dominant-negative effect of the mutant protein and restore normal function to cells in vitro. Whereas the effects of siRNA are transient, lasting only as long as the siRNA is present in the cell at high enough concentrations; CRISPR/Cas9 gene editing offers the opportunity to permanently modify the mutant allele.

The discovery of a simple endogenous bacterial system for catalytically cleaving double-stranded DNA has revolutionized the field of therapeutic gene editing. The Type II Clustered Regularly Interspaced. Short Palindromic Repeats (CRISPR)/CRISPR associated protein 9 (Cas9) is a programmable RNA guided endonuclease, which has recently been shown to be effective at gene editing in mammalian cells (Hsu P D, Lander E S, Zhang F. Development and applications of CRISPR-Cas9 for genome engineering. Cell 2014; 1:57: 1262-1278). This highly specific and efficient RNA-guided DNA endonuclease may be of therapeutic importance in a range of genetic diseases. The CRISPR/Cas9 system relies on a single catalytic protein, Cas9 that is guided to a specific DNA sequence by 2 RNA molecules; the tracrRNA and the crRNA (Hsu P D, Lander E S, Zhang F. Development and applications of CRISPR-Cas9 for genome engineering. Cell 2014; 157: 1262-1278). Combination of the tracrRNA/crRNA into a single guide RNA molecule (sgRNA) (Shalem O, Sanjana N E, Hartenian E, Shi X, Scott D A, Mikkelsen T S et al. Genome-scale CRISPR-Cas9 knockout screening in human cells. Science 2014; 343: 84-87; Wang T, Wei J J, Sabatini D M, Lander E S. Genetic screens in human cells using the CRISPR-Cas9 system. Science 2014; 343: 80-84) has led to the rapid development of gene editing tools potentially specific for any target within the genome. Through the substitution of a nucleotide sequence within the sgRNA, to one complimentary to a chosen target, a highly specific system may be generated in a matter of days. One caveat of this system is that the endonuclease requires a protospacer adjacent motif (PAM), located immediately at the 3′ end of the sgRNA binding site, This PAM sequence is an invariant part of the DNA target but not present in the sgRNA, while its absence at the 3′ end of the genomic target sequence results in the inability of the Cas9 to cleave the DNA target (Westra E R, Semenova E, Datsenko K A, Jackson R N, Wiedenheft B, Severinov K et al. Type CRISPR-cas systems discriminate target from non-target DNA through base pairing-independent PAM recognition. PLoS Genet 2013; 9: e1003742).

In one aspect, the present disclosure describes the potential of an allele-specific CRISPR/Cas9 system for corneal dystrophies, for example, on a dominant-negative mutation in KRT12 (encoding keratin 12, K12), Leu132Pro (c. 395 T>C), which results in Meesmann epithelial conical dystrophy (MFCD; OMIM:122100) (Liao H, Irvine A D, Macewen C J, Weed K H, Porter L, Corden L D et al. Development of allele-specific therapeutic siRNA in Meesmann epithelial corneal dystrophy. PLoS One 2011; 6: e28582). Interestingly, as shown herein, this mutation results in the manifestation of a novel Streptococcus pyogenes PAM, not present in the wild-type allele. In some embodiments, the present disclosure shows that an allele-specific cleavage of the mutant allele may be induced by incorporating nucleotides at the 5′ end of this novel PAM into an sgRNA. In a heterozygous cell, this double-strand break may either lead to non-homologous end joining (NHEJ), which may result in a frameshift and the manifestation of a premature stop codon, or homology-directed repair where recombination with the wild-type allele directs repair of the mutant sequence. In the case of KRT12, for example, both outcomes could be considered a therapeutic success; either expression of the dominant-negative mutant K12 protein is abolished by NHEJ, which is tolerated, as KRT12 has been shown not to demonstrate haploinsufficiency (Kao W W, Liu C Y, Converse R L, Shiraishi A, Kao C W, Ishizaki M et al. Keratin 12-deficient mice have fragile corneal epithelia. Invest Ophthalmol Vis Sci 1996; 37: 2572-2584), or the mutant allele is repaired by homology-directed repair, resulting in the repair of the K12-Leu132Pro allele.

### SUMMARY OF THE INVENTION

In one aspect, the present disclosure is related to single guide RNA (sgRNA). In some embodiments, the sgRNA comprises (i) CRISPR targeting RNA (crRNA) sequence and (ii) a trans-activating crRNA (tracrRNA) sequence. In some embodiments, the crRNA sequence and tracrRNA sequence do not naturally occur together. In some embodiments, the crRNA sequence has the nucleotide sequence having at least about 80, 85, 90, 95 or 100% sequence identity with a nucleotide sequence selected from the group consisting of SEQ ID NO: (10+4n), in which n is an integer from 0 to 221. In additional embodiments, the tracrRNA sequence comprises a nucleotide sequence having at least about 80, 85, 90, 95 or 100% sequence identity with the sequence of SEQ ID NO: 2 or 6.

In another aspect, the present disclosure is related to an sgRNA pair designed for CRISPR/Cas9 system, the sgRNA pair comprising (i) a first SgRNA comprising (a) a first crRNA sequence for a first protospacer adjacent motif (PAM) generating mutation or singie-nucieotide polymorphism (SNP) at 3′-end side of a disease-causing mutation or SNP in cis, and (b) a tracrRNA sequence, in which the first crRNA sequence and the tracrRNA sequence do not naturally occur together, (ii) a second sgRNA comprising (a) a second crRNA guide sequence for a second PAM generating mutation or SNP at 5′-end side of the disease-causing mutation or SNP in cis; (b) a tracrRNA sequence, in which the second crRNA sequence and the tracrRNA sequence do not naturally occur together. In some embodiments, the CRISPR/Cas9 system is for preventing, amelioratng or treating corneal dystrophies In some embodiments, the PAM generating mutations or SNPs are located in TGFBI gene. In further embodiments, the PAM generating mutations or SNPs are in introns of TGFBI gene. For example, the PAM generating mutations or SNPs are in adjacent introns of TGFBI gene, and the disease-causing mutation or SNP may be in exon in between the acjacent introns as shown in FIG. 16. In yet further embodiments, at least one of the first and second crRNA sequences comprises a nucleotide sequence selected from the group consisting of sequences listed in FIGS. 19-35; and or at least one of the first and second crRNA sequences comprises a nucleotide sequence selected from the group consisting of sequences listed in Table 2.

In another aspect, the present disclosure is related to engineered Clustered Regularly Interspaced Short Palindromic Repeats (CRISPR)/CRISPR associate protein 9 (Cas9) systems comprising at least one or two vectors comprising a nucleotide molecule encoding Cas9 nuclease and the sgRNA described herein, or at least one, two, or three different vectors comprising nucleotide molecules encoding Cas9 nuclease and the sgRNA pair described herein. In some embodiments, the Cas9 nuclease and the sgRNA do not naturally occur together. In some embodiments, the Cas9 nuclease described herein may be an enhanced Cas9 nuclease described in Slaymaker et al. 2016 Science, 351(6268), 84-88. In additional embodiments, the Cas9 nuclease is from Streptococcus. In yet additional embodiments, the Cas9 nuclease is from Streptococcus pyogenes (Spy), Streptococcus dysgalactiae, Streptococcus canis, Streptococcus equi, Streptococcus iniae, Streptococcus phocae, Streptococcus psceudoporcinus, Streptococcus oralis, Streptococcus pseudoporcinus, Streptococcus infantarius, Streptococcus mutans, Streptococcus agalactie, Streptococcus caballi, Streptococcus equinus, Streptococcus sp. oral taxon, Streptococcus mitis, Streptococcus gallolyticus, Streptococcus gordonii, Streptococcus pasteurtianus, or variants thereof. In additional embodiments, the Cas9 nuclease is from Staphylococcus. In yet additional embodiments, the Cas9 nuclease is from Staphylococcus aureus, S. simiae, S. auricularis, S. carnosus, S. condimenti, S. massiliensis, S. piscifermentans, S. simulans, S capitis, S. caprae, S. epidermidis, S. saccharolyticus, S. devriesei, S. haemolyticus, S. hominis, S. agnetis, S. chromogenes, S. felis, S. delphini, S. hyicus, S. intermedius, S. lutrae, S. microti, S. muscae, S. pseudintermedius, S. rostri, S. schleiferi, S. lugdunensis, S. arlettae, S. cohnii, S. equorum, S. gallinarum, S. kloosii, S. leei, S. nepalensis, S. saprophyticus, S. succinus, S. xylosus, S. fleuretti, S. lentus, S. sciuri, S. stepanovicii, S. vitulinus, S. simulans, S. pasyeuri, S. warneri, of variants thereof. In further embodiments, the Cas9 nuclease comprises an amino acid sequence having at least about 60% sequence identity with an amino acid sequence selected from the group consisting of SEQ ID NO: 4 or 8. In yet further embodiments, the nucleotide molecule encoding Cas9 nuclease comprises a nucleotide sequence having at least about 60% sequence identity with a nucleolide sequence selected from the group consisting of SEQ ID NO. 3 or 7. In some embodiments, the CRISPR/Cas9 system or the vector described herein excludes or further comprises a repair nucleotide molecule and or at least one nuclear localization signal (NLS). In additional embodiments, the sgRNA and the Cas9 nuclease are included on the same vector or on different vectors.

In another aspect, the present disclosure is related to methods of altering expression of at least one gene product comprising introducing the engineered CRISPR/Cas9 system described herein into a cell containing and expressing a DNA molecule having a target sequence and encoding the gene product. In some embodiments, the engineered CRISPR/Cas9 system comprises (a) a first regulatory element operably linked to the sgRNA that hybridizes with the larget sequence, and (b) a second regulatory element operably linked to the nucleotide molecule encoding Cas9 nuclease, wherein components (a) and (b) are located on a same vector or different vectors of the system, the sgRNA targets the target sequence, and the Cas9 nuclease cleaves the DNA molecule. The target sequence may be a nucleotide sequence complementary to the nucleotide sequence adjacent to the 5′-end of a protospacer adjacent motif (PAM). In additional embodiments, the cell is a eukaryotic cell, or a mammalian or human cell. In some embodiments, the sgRNA comprises a nucleolide sequence adjacent to the 5′ end of a PAM recognized by the Cas9 nuclease. In additional embodiments, the sgRNA has from 16 to 25 nucleotide sequence length, or 16, 17, 18, 19, 20, 21, 22, 23, 24 or 25 nucleotides.

In another aspect, the present disclosure is related to methods of preventing, ameliorating, or treating a disease associated with single-nucleotide polymorphism (SNP) in a subject comprising altering expression of the gene product of the subject as described herein, wherein the DNA molecule comprises a mutant sequence. In some embodiments, the DNA molecule may compnse at least one, two, three, four or more SNP or mutant sites, and the method described herein alters the expression of the gene product related to at least one, two, three, four or more of the SNP or mutant sites.

In another aspect, the present disclosure is related to methods of preventing, ameliorating, or treating corneal dystrophy associated with a gene mutation or SNP in a subject, comprising administering to the subject an engineered CRISPR/Cas9 system comprising at least one or two vectors comprising (i) a nucleotide molecule encoding Cas9 nuclease described herein, and (ii) sgRNA described herein, wherein the sgRNA hybridizes to a nucleotide sequence complementary to or comprises a target sequence adjacent to the 5′-end a protospacer adjacent motif (PAM) site, and the target sequence or the PAM comprises a mutation or SNP site. In some embodiments, the sgRNA comprises a nucleotide sequence having at least about 75, 80, 85, 90, 95 or 100% sequence identity with the target sequence. In some embodiments, the Cas9 nuclease and the sgRNA do not naturally occur together. In additional embodiments, the PAM comprises the mutation or SNP site.

In some embodiments, a mutant sequence comprising the disease-causing gene mutation or SNP encodes a mutant protein selected from the group consisting of (i) mutant TGFBI proteins comprising Leu509Arg, Arg666Ser, Gly623Asp, Arg555Gln, Arg124Cys, Val505Asp, Ile522Asn, Leu569Arg, His572Arg, Arg496Trp, Pro501Thr, Arg514Pro, Phe515Leu, Leu518Pro, Leu518Arg, Leu527Arg, Thr538Pro, Thr538Arg, Val539Asp, Phe540del, Phe540Ser, Asn544Ser, Ala546Thr, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Val624 Val625del, Val624Met, Val625Asp, His626Arg, His626Pro, Val627SerfsX44, Thr629_Asn630insAsnValPro, Val631Asp, Arg666Ser, Arg555Trp, Arg124Ser, Asp123delins, Arg124His, Arg124Leu, Leu509Pro, Leu103_Ser104del, Val113Ile, Asp123His, Arg124Leu, and/or Thr125_Glu126del; (ii) mutant KRT3 proteins with Glu498Val, Arg503Pro, and/or Glu509Lys; (iii) mutant KRT12 proteins with Met129Thr, Met129Val, Gln130Pro, Leu132Pro, Leu132Va, Leu132His, Asn133Lys, Arg135Gly, Arg135Ile, Arg135Thr, Arg135Ser, Ala137Pro, Leu140Arg, Val143Leu, Val143Leu, Lle391_Leu399dup, Ile 426Val, Ile426Ser, Tyr429Asp, Tyr429Cys, Arg430Pro, and/or Leu433Arg; (iv) mutant GSN proteins with Asp214Tyr, and (v) mutant UBIAD1 proteins with Ala97Thr, Gly98Ser, Asn102Ser, Asp112Asn, Asp112GlyAsp118Gly, Arg119Gly, Leu121Val, Leu121Phe, Val122Glu, Va1122G1y, Ser171Pro, Tyr174Cys, Thr175Ile, Gly177Arg, Lys181Arg, Gly186Arg, Leu188His, Asn232Ser, Asn233His, Asp236Glu, and/or Asp240Asn. In yet further embodiments, the method according to any one of claims 14-25, wherein the subject is human, animal or mammal.

In some embodiments, the mutant sequence comprising the SNP site encodes the mutant TGFBI protein comprising Arg514Pro, and the engineered CRISPR/Cas9 system comprises the sgRNA comprising GAACTAATTACCATGCTAAA (SEQ ID NO: 897). In some embodiments, the mutant sequence comprising the SNP site encodes the mutant TGFBI protein comprising Leu518Arg, and the engineered CRISPR/Cas9 system comprises the sgRNA comprising GAGACAATCGCTTTAGCATG (SEQ ID NO: 898). In some embodiments, the mutant sequence comprising the SNP site encodes the mutant TGFBI protein comprising Leu509Arg, and the engineered CRISPR/Cas9 system comprises the sgRNA comprising SEQ ID NO: 186. In some embodiments, the mutant sequence comprising the SNP site encodes the mutant TGFBI protein comprising Leu527Arg, and the engineered CRISPR/Cas9 system comprises the sgRNA comprising SEQ ID NO: 474. In some embodiments, the mutant sequence comprising the SNP site encodes the mutant TGFBI protein comprising Arg124Cys, and the engineered CRISPR/Cas9 system comprises the sgRNA comprising any nucleotide sequence of SEQ ID NOs: 58, 54, 50 and 42. In some embodiments, the mutant sequence comprising the SNP site encodes the mutant TGFBI protein comprising Arg124His, and the engineered CRISPR/Cas9 system comprises the sgRNA comprising any nucleotide sequence of SEQ ID NOs: 94, 90, 86, 82, 78, 74 and 70. In some embodiments, the mutant sequence comprising the SNP site encodes the mutant TGFBI protein comprising Arg124His, and the engineered CRISPR/Cas9 system comprises the sgRNA comprising SEQ ID NO: 86 or 94. In some embodiments, the mutant sequence comprising the SNP site encodes the mutant TGFBI protein comprising Arg124Leu, and the engineered CRISPR/Cas9 system comprises the sgRNA comprising any nucleotide sequence of SEQ ID NOs: 114, 110, 106 and 98. In sonic embodiments, the mutant sequence comprising the SNP site encodes the mutant TGFBI protein comprising Arg555Gln, and the engineered CRISPR/Cas9 system comprises the sgRNA comprising any nucleotide sequence of SEQ ID NOs: 178, 174, 170, 166, 162 and 158. In sonie embodiments, the mutant sequence comprising the SNP site encodes the mutant TGFBI protein comprising Arg555Trp, and the engineered CRISPR/Cas9 system comprises the sgRNA comprising any nucleotide sequence of SEQ ID NOs: 146, 142, 138, 134, 130 and 126. In some embodiments, the mutant sequence comprising the SNP site encodes the mutant TGFBI protein comprising Leu527Arg, and the engineered CRISPR/Cas9 system comprises the sgRNA comprising any nucleotide sequence of SEQ ID NOs: 146, 142, 138, 134, 130 and 126.

In another aspect, the present disclosure is related to methods of preventing, ameliorating, or treating corneal dystrophy associated with a gene mutation or SNP in a subject, comprising administering to the subject an engineered CRISPR/Cas9 system comprising at least one or two vectors comprising (i) a nucleotide molecule encoding Cas9 nuclease described herein, and (ii) sgRNA described herein, wherein the sgRNA hybridizes to a first target sequence complementary to a second target sequence adjacent to the 5′-end a protospacer adjacent motif (PAM) site, and the first target sequence or the PAM comprises the mutation or SNP site. In another aspect, the present disclosure is related to methods of preventing, ameliorating, or treating corneal dystrophy associated with a gene mutation or single-nucleotide polymorphism (SNP) in a subject, comprising administering to the subject an engineered CRISPR/Cas9 system comprising at least one vector comprising (i) a nucleotide molecule encoding Cas9 nuclease; (ii) a first CRISPR targeting RNA (crRNA) sequence that hybridizes to a nucleotide sequence complementary to a first target sequence, the first target sequence being adjacent to the 5′-end of a first protospacer adjacent motif (PAM) at 3′-end side of a disease-causing mutation or SNP in cis, wherein the first target sequence or the first PAM comprises a first ancestral mutation or SNP site, (iii) a second crRNA sequence that hybridizes to a nucleotide sequence complementary to a second target sequence, the second target sequence being adjacent to the 5′-end of a second PAM at 5′-end side of a disease-causing mutation or SNP in cis, wherein the second target sequence or the second. PAM comprises a second ancestral mutation or SNP site, wherein the at least one vector does not have a nucleotide molecule encoding Cas9 nuclease and a crRNA sequence that naturally occur together. In some embodiments, the PAM generating mutations or SNPs are in TGFBI gene, for example, in introns of TGFBI gene. In additional embodiments, at least one of the first and second crRNA sequences comprises a nucleotide sequence selected from the group consisting of sequences listed in FIGS. 19-35; and/or at least one of the first and second crRNA sequences comprises a nucleotide sequence selected from the group consisting of sequences listed in Table 2. In yet additional embodiments, wherein the first PAM comprises the first mutation or SNP site and/or the second PAM comprises the second mutation or SNP site. In further embodiments, the first crRNA sequence comprises the first target sequence, and/or the second crRNA sequence comprises the second target sequence. In yet further embodiments, the crRNA is from 17 to 24 nucleotide long. In some embodiments, the first and second PAMs are both from Streptococcus or Staphylococcus. In additional embodiments, a mutant sequence comprising the disease-causing mutation or SNP encodes a mutant protein selected from the group consisting of mutant TGFBI proteins comprising Leu509Arg, Arg666Ser, Gly623Asp, Arg555Gln, Arg124Cys, Val505Asp, Il522Asn, Leu569Arg, His572Arg, Arg496Trp, Pro501Thr, Arg514Pro, Phe515Leu, Leu518Pro, Leu518Arg, Leu527,Arg, Thr538Pro, Thr538Arg, Val539Asp, Phe540del, Phe540Ser, Asn544Ser, Ala546Thr, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Val624_Val625del, Val624Met, Val625Asp, His626Arg, His626Pro, Val627SerfsX44, Thr629_Asn630insAsnValPro, Val631Asp, Arg666Ser, Arg555TrpArg124Ser, Asp123delins, Arg124His, Arg124Leu, Leu509Pro, Leu103_Ser104del, Val113Ile, Asp123His, Arg124Leu, and/or Thr125_Glu126del.

In yet additional embodiments, the PAM consists of a PAM selected from the group consisting of NGG and NNGRRT, wherein N is any of A, T, G, and C, and R is A or G. In further embodiments, the administering comprises introducing the engineered CRISPR/Cas9 system into a cornea (e.g., corneal stroma) of the subject, for example, by injecting the engineered CRISPR/Cas9 system into a cornea (e.g., conical stroma) of the subject and/or by introducing the engineered CRISPR/Cas9 system into a cell containing and expressing a DNA molecule having the target sequence.

In some embodiments, the corneal dystrophy is selected from the group consisting of Epithelial basement membrane dystrophy (EBMD), Meesmann corneal dystrophy (MECD), ThielBehnke corneal dystrophy (TBCD), Lattice conical dystrophy (LCD), Granular corneal dystrophy (GCD), and Schnyder corneal dystrophy (SCD). In additional embodiments, the SNP site is located in a gene selected from the group consisting of TGFBI, KRT3, KRT12, GSN, and UBIAD1 prenyltransferase domain containing 1 (UBIAD1).

In some embodiments, the CRISPR/Cas9 system and the methods using the same described herein may alter mutant sequences at a plurality of SNP sites or ancestral SNPs.

In another aspect, the present disclosure is related to methods of treating corneal dystrophy in a subject in need thereof, comprising:(a) obtaining a plurality of stem cells comprising a nucleic acid mutation in a corneal dystrophy target nucleic acid from the subject; (b) manipulating the nucleic acid mutation in one or more stem cells of the plurality of stem cells to correct the nucleic acid mutation, thereby forming one or more manipulated stem cells; (c) isolating the one or more manipulated stem cells; and (d) transplanting the one or more manipulated stem cells into the subject, wherein manipulating the nucleic acid mutation in the one or more stem cells of the plurality of stem cells includes performing any of the methods of altering expression of a gene product or of preventing, ameliorating, or treating a disease associated with mutation or SNP in a subject as described herein.

### BRIEF DESCRIPTION OF THE DRAWINGS

FIG. 1 illustrates exemplary design of sgRNAs for targeting wild-type and mutant keratin 12 (K12) alleles. An sgRNA to use the SNP-derived PAM found on the K12-L132P allele was designed (red). This PAM is absent from the wild-type allele. A second sgRNA to target both wild-type and mutant K12 alleles (green) vas also designed and used as a positive control.

FIG. 2 illustrates evaluation of allele specificity and potency of sgK12LP using exogenous expression constructs. Exogenous expression constructs for wild-type and mutant K12 were employed to test the allele-specificity and potency of sgK12LP. (a) A dual luciferase assay demonstrated the allele-specificity of the sgK12LP plasmid, whereas potency was shown to be comparable to that of the sgK12 construct. N=8 (b) Western blotting further demonstrated these attributes with a noticeable reduction in K12-L132P protein in cells treated with sgK12LP in comparison with cells treated but expressing K12 wild-type protein. β-Actin was used as a loading control. (c) Quantitative reverse transcriptase-PCR for total K12 in cells expressing both wild-type and mutant alleles demonstrated a knockdown in mRNA expression. N=4 (d) Allele proportions of this mRNA knockdown were then quantified by pyrosequencing, confirming an allele knockdown of the mutant allele in cells co-expressing both KRTI 2 alleles and treated with sgK12LP. N=4, *P<0.05, ***P<0.001.

FIG. 3 illustrates sgK12LP-induced NHEJ in vivo. GFP expression was observed in the corneal epithelium of mice at 24 h post intrastromal injection, demonstrating the efficacy of an intrastromal plasmid injection for transfecting the corneal epithelium (a; N=2), No GFP expression was observed at 48 h post injection. Sequencing of the gDNA from human K12-L132P heterozygous mice injected with the sgK12LP construct demonstrated large deletions and the induction of NHEJ due to cleavage of the KRT12-L132P allele. Of 13 clones sequenced, 5 were found to have undergone NHEJ (b).

FIG. 4 shows results using SNP derived PAM guide RNAs designed for TGFBI mutations R514P (A), L518R (B), L509R (C), L527R (D) and luciferase expression was used to assess wild type and mutant allele expression. A positive control (sgWT) guide was designed to cut both wild type (WT, blue bar) and mutant type (MUT, red bar) allele and as shown above cuts both alleles as expected. The Guide used for L518R (sgMut) shows the greatest allele specificity with minimal cutting of the WT allele (blue bar). The negative control guide (sgNSC) as expected did not cut either of the WT nor MUT DNA.

In FIG. 5, items A-E show results using mutant allele specific guide RNAs designed for R124 and R555 TGFBI mutations and luciferase expression was used to assess wild type and mutant allele expression. The assay was conducted with guides of different lengths, ranging from 16 mer to 22 mer. In addition to the guide length, the addition of a double guanine to the 5′ end of the guide to help improve specificity was also assessed. The blue bars represent WT TGFBI sequence, and the orange bars depict mutant TGFBI sequence. The mutant guides cut with varying efficiencies based on the length of the guides (FIG. 5, items A-E). For R124 (FIG. 5, items A, B and C), assays show an allele-specific trend, with the mutant guide preferentially targeting mutant sequence (orange bars further reduced in comparison to blue bars).

In FIG. 5, item F shows the improved specificity when a R124H mutant 20 mer guide is tested with an enhanced Cas9 nuclease engineered to reduce non-target binding.

In FIG. 5, item G shows the fragment analysis from in vitro cleavage with Cas9 to confirm that the DNA has been cleaved. Cleavage templates were prepared for the wild-type and mutant sequence for each of the 6 common TGFBI mutations (e.g., R124C, R124H, R124L , R555Q, R555W, and L527R). Guide RNA molecules (20 and 18 nucleotides) containing wild-type and mutant sequence were designed and synthesized. Cleavage templates were then digested in vitro with a Cas9-sgRNA complex and fragment analysis was performed on an agarose gel (FIG. 5, item G, (a)-(f). Fragment analysis of the R124C cleavage reaction (FIG. 5, item G, (a)) shows results comparable with those of the dual luciferase assay (FIG. 5, item A). Analysis of the cleavage reactions for both R124H and R124L (FIG. 5, item G, (b) and (c)) again show similar findings to that of the dual luciferase assay (FIG. 5, items B and C) and the results concur between the two very different assays. Examination of the R555Q and R555W cleavage reactions (FIG. 5, item G, (d) and (e)) again indicate comparability to the dual luciferase assay (FIG. 5, items D and E). Analysis of the cleavage reactions for L527R (FIG. 5, item G, (f) shows varying cutting efficiencies based on the length of the guides.

In FIG. 6, (A) shows an exemplary single guide RNA (sgRNA) target sequence (shown highlighted in purple) specific for Luc2 and designed to target the 5′ region of the Luc2 gene. Designing the guide to bind in the 5′ region of the Luc2 gene increased the likelihood of inducing a frame-shifting deletion and knockout luciferase (Luc2) activity by generating a premature termination codon in the targeted DNA. (B) shows results obtained after adding this Luc2 targeting guide to cells expressing luciferase and measuring gene editing based on luciferase expression. Some cells were untreated (unT) and other cells were treated with a non-specific negative control guide RNA (sgNSC) which would not bind to the DNA in the cells and also the test guide against Luc2 which is sgLuc2P.

FIG. 7 demonstrates in vivo in the mouse corneal epithelium that CRISPR Cas9 gene editing can cut and lower the expression of the target gene and this result in less protein being expressed from that gene. The heat map of luciferase is representative of level of protein expression where black reflects no expression, blue reflects low expression, and red reflects high expression for Luc2 protein.

FIG. 8 illustrates CRISPR/Cas9 system described in F. Ran et al., Nat. Protoc. 2013, 8(11) 2281-2308. The Cas9 nuclease from S. pyogenes (in yellow) is targeted to genomic DNA (shown for example is the human EMX1 locus) by an sgRNA consisting of a 20-nt guide sequence (blue) and a scaffold (red). The guide sequence pairs with the DNA target (blue bar on top strand), directly upstream. of a requisite 5′-NGG adjacent motif (PAM, pink). Cas9 mediates a DSB ˜3 bp upstream of the PAM (red triangle).

FIG. 9 illustrates CRISPR/Cas9 system described in F. Ran et al., Nature 2015, 520(7546) 186-91, including schematic of Type II CRISPR-Cas loci and sgRNA from eight bacterial species. Spacer or “guide” sequences are shown in blue, followed by direct repeat (gray). Predicted tracrRNAs are shown in red, and folded based on the Constraint Generation RNA folding model.

FIG. 10 also illustrates CRISPR/Cas9 system described in F. Ran et al., Nature 2015, 520(7546):186-91 The figure shows optimization of SaCas9 sgRNA scaffold in mammalian cells, a, Schematic of the Staphylococcus aureus subspecies aureus CRISPR locus, b. Schematic of SaCas9 sgRNA with 21-nt guide, crRNA repeat (gray), tetraloop (black) and tracrRNA (red) The number of crRNA repeat to tracrRNA anti-repeat base-pairing is indicated above the gray boxes. SaCas9 cleaves targets with varying repeat: anti-repeat lengths in c, HLK 293FT and d, Hcpal-6 cell lines, (n=3, error bars show S.E.M.)

FIG. 11 illustrates exemplary vectors for CRISPR/Cas9 system, including pSpCas9(BB))-2A-Puro (PX459) using Streptococcus pyogenes Cas9 nuclease.

FIG. 12 illustrates exemplary vectors for CRISPR/Cas9 system, pX601-AAV-CMV::NLS-SaCas9-NLS-3xHA-bGHpA;U6::Bsal-sgRNA using Staphylococcus aureus.

FIG. 13 illustrates exemplary sgRNA sequence, nucleotide and ammo acid sequences of Cas9 nuclease from Streptococcus pyogenes (Spy) and Staphylococcus aureus (Sau).

FIG. 14 illustrates exemplary design for HDR-mediated repair of a Meesmann corneal dystrophy (MECD)-associated KHT12 mutations that are tightly clustered with one pair of sgRNAs to direct Cas9 cleavage. The repair oligo (ssODN) shown in FIG. 14 is for L132P, but would also work for the other mutations in the cluster. The site of the mutation and repair is shown with an asterisk. The two arrowheads show nucleotide changes in the repair oligo that will introduce synonymous changes in the repaired allele that will prevent further cutting by the Cas9 as they recode the PAM site.

FIG. 15 illustrates all SNPs in TGFBI with a MAF of >10% that generate a novel PAM. The numbered boxes indicate the exons within TGFBI. The hotspots in TGFBI, where multiple disease-causing mutations are found, are shown by the red boxes. The blue arrows indicate the position of a SNP thai generates a novel PAM. The novel PAM is shown for each arrow, with the required variant highlighted in red.

FIG. 16 illustrates an exemplary embodiment in which a sgRNA utilizing a flanking SNP novel PAM is designed in the first intron. Additionally, a sgRNA common to both the wild-type and mutant allele is designed in the second intron. In the wild-type allele the single sgRNA causes NHEJ in the second intron, which has no functional effect. However, in the mutant allele, the sgRNA utilizing the flanking SNP derived PAM and the common sgRNA result in a large deletion that results in a knockout of the mutant allele.

FIG. 17 depicts experimental results from using an exemplary lymphocyte cell line derived from a patient with a R124H Avellino corneal dystrophy mutation that was nucleofected with CRISPR/Cas9. The guide utilized the novel PAM that is generated by the rs3805700 SNP. This PAM is present on the same chromosome as the patients R124H mutation but does not exist on the wild-type chromosome. Following cell sorting, single clones were isolated to determine whether indels had occurred. Six of the single clones had the unedited wild-type chromosome, indicating stringent allele-specificity of this guide. Four of the isolated clones had the mutant chromosome, and three of these exhibited edits indicating a 75% editing efficiency of the mutant chromosome. Two of the three clones exhibited indels that are frame-shifting. Therefore, at least 66.66% of the edits induced gene disruption.

FIG. 18 illustrates exemplary target sites, guide sequences and their complementary sequences.

FIG. 19 illustrates exemplary target sequences including SNP sites associated with corneal dystrophies.

FIGS. 20-35 illustrate exemplary common guides in intronic regions of TGFBI gene.

### DETAILED DESCRIPTION OF THE INVENTION

As used throughout, ranges are used as shorthand for describing each and every value that is within the range. Any value within the range can be selected as the terminus of the range. In addition, all references cited herein are hereby incorporated by reference in their entireties for all purposes. In the event of a conflict in a definition in the present disclosure and that of a cited reference, the present disclosure controls.

In one aspect, the present disclosure is related to single guide RNA (sgRNA), for example, including sgRNAs designed for CRISPR/Cas9 system for preventing, ameliorating or treating corneal dystrophies. The sgRNA may be artificial, man-made, synthetic, and/or non-naturally occurring. In some embodiments, the sgRNA comprises (i) CRISPR targeting RNA (crRNA) sequence and (ii) a trans-activating crRNA (tracrRNA) sequence, which also may be called “sgRNA scaffold.” In some embodiments, the crRNA sequence and tracrRNA sequence do not naturally occur together. As used herein, the term “sgRNA” may refer to a single guide RNA containing (i) a guide sequence (crRNA sequence) and (ii) a Cas9 nuclease-recruiting sequence (tracrRNA). The exemplary guide sequences include those disclosed in FIGS. 18-19. The crRNA sequence may be a sequence that is homologous to a region in your gene of interest and may direct Cas9 nuclease activity. The crRNA sequence and tracrRNA sequence do not naturally occur together. The sgRNA may be delivered as RNA or by transforming with a plasmid with the sgRNA-coding sequence (sgRNA gene) under a promoter.

In some embodiments, the sgRNA or the crRNA hybridizes to at least a part of a target sequence (e.g., target genome sequence), and the crRNA may have a complementary sequence to the target sequence. In some embodiments, the target sequence herein is a first target sequence that hybridizes to a second target sequence adjacent to a PAM site described herein. In some embodiments, the sgRNA or the crRNA may comprise the first target sequence or the second target sequence. “Complementarily” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry And Molecular Biology-Hybridization With Nucleic Acid Probes Part 1, Second Chapter “Overview of principles of hybridization and the strategy of nucleic acid probe assay”, Eisevier, N.Y. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. In some embodiments, the crRNA sequence has the nucleotide sequence having at least about 80, 85, 90, 95 or 100% sequence identity h a nucleotide sequence selected from the group consisting of SEQ ID NO: (10+4n), in which n is an integer from 0 to 221. As used herein, the term “about” may refer to a range of values that are similar to the stated reference In certain embodiments, the term “about” refers to a range of values that fall within 15, 10, 9, 8,7, 6, 5, 4, 3, 2, 1 percent or less of the stated reference value. In some embodiments, the crRNA sequence has the nucleotide sequence having one, two, three, four or five nucleotide additions, deletions and/or substitutions from a nucleotide sequence selected from the group consisting of SEQ ID NO: (10+4n), in which n is an integer from 0 to 221. Such additions, deletions and/or substitutions may be at the 3′-end or 5′-end of the nucleotide sequence. In additional embodiments, the crRNA or the guide sequence is about 17, 18, 19, 20, 21, 22, 23 or 24 nucleotide long. In further embodiments, the crRNA excludes crRNA sequences having the nucleotide sequences of SEQ ID NO: 10. In yet further embodiments; the crRNA excludes crRNA sequences hybridizing to a nucleotide sequence comprising a SNP resulting in L132P mutation in keratin 12 protein. In yet further embodiments; the crRNA excludes crRNA sequences hybridizing to a nucleotide sequence comprising a SNP resulting in a mutation in keratin 12 protein.

In some embodiments, tracrRNA provides a hairpin structure that activates Cas9 for opening up the dsDNA for binding of the crRNA sequence. The tracrRNA may have a sequence complementary to the palindromic repeat. When the tracrRNA hybridizes to the short palindromic repeat, it may trigger processing by the bacterial double-stranded RNA-specific ribonuclease, RNase III. In additional embodiments, the tracrRNA may have SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci may differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In certain embodiments, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). The tracrRNA sequence may be any sequence for tracrRNA for CRISPR/Cas9 system known in the art. In additional embodiments, the tracrRNA comprises a nucleotide sequence having at least about 70, 75, 80, 85, 90, 95 or 100% sequence identity with the nucleotide sequence of SEQ ID NO: 2 and 6. The tracrRNA sequence may be any sequence for tracrRNA for CRISPR/Cas9 system known in the art. Exemplary CRISPR/Cas9 systems, sgRNA, crRNA and tracrRNA, and their manufacturing process and use are disclosed in U.S. Pat. No. 8,697,359, U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, 20140080216, and WO2016049024, all of which are incorporated herein by their entirety.

In another aspect, the present disclosure is related to an oligonucleotide pair to be inserted in a vector for CRISPR/Cas9 system, in which the oligonucleotide pair comprises a primer comprising a crRNA sequence described herein. The primer may further comprise a locator sequence of 2, 3, 4, 5 or 6 nucleotides adjacent to the crRNA sequence, in which the locator sequence does not occur naturally adjacent to the crRNA sequence. In some embodiments, the present disclosure is related to an oligonucleotide pair to be incorporated in a vector for encoding crRNA for CRISPR/Cas9 system, such as pSpCas9(BB)-2A-Puro (PX459) and pX601-AAV-CMV::NLS-SaCas9-NLS-3x1-1A-bGHpA;U6::Bsal-sgRNA, in which the oligonucleotide pair comprises a primer comprising the nucleotide sequence of SEQ ID NO: (10+4n), in which n is an integer from 0 to 221. In additional embodiments, the oligonucleotide pair comprises a first primer having the nucleotide sequence of SEQ ID NO: X, and the second primer having the nucleotide sequence of SEQ ID NO: Y, in which X is 11+4n, Y is 12+4n, and n is an integer from 1 to 221. In some embodiments, the crRNA comprises a nucleotide sequence selected from the group consisting of SEQ ID NO: 58, 54, 50, 42, 94, 90, 86, 82, 78, 74, 70, 114, 100, 106, 98, 178, 174, 170, 166, 162, 158, 146, 142, 138, 134, 130 and 126

In another aspect, the present disclosure is related to engineered Clustered Regularly Interspaced Short Palindromic Repeats (CRISPR)/CRISPR associate protein 9 (Cas9) systems comprising at least one vector comprising a nucleotide molecule encoding Cas9 nuclease and the sgRNA described herein. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In some embodiments, the Cas9 nuclease and the sgRNA do not naturally occur together.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence tracrRNA or an active partial tracrRNA), a tracr-mate sequence (encompassing a “direct repeat” and a tracrRNA-processed partial direct repeat in the context of an endogenous CRISPR system), a guide sequence (also referred to as “crRNA” herein, or a “spacer” in the context of an endogenous CRISPR system), and/or other sequences and transcripts from a CRISPR locus. As described above, sgRNA is a combination of at least tracrRNA and crRNA. In some embodiments, one or more elements of a CRISPR system is derived from a type II CRISPR system. In sonic embodiments, one or more elements of a CRISPR system is derived from a particular organism comprising an endogenous CRISPR system, such as Streptococcus pyogenes or Staphylococcus aureus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” may refer to a sequence to which a guide sequence is designed to have complementarity, where hybridization between a target sequence and a guide sequence promotes the formation of a CRISPR complex, or as shown in FIG. 8, the “target sequence” may refer to a sequence adjacent to a PAM site, which the guide sequence comprises. Full complementarity is not necessarily required, provided there is sufficient complementarity to cause hybridization and promote formation of a CRISPR complex. In this disclosure, “target site” refers to a site of the target sequence including both the target sequence and its complementary sequence, for example, in double stranded nucleotides. In some embodiments, the target site described herein may mean a first target sequence hybridizing to sgRNA or crRNA of CRISPR/Cas9 system, and/or a second target sequence adjacent to the 5′-end of a PAM. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast.

In some embodiments, the Cas9 nucleases described herein are known; for example, the amino acid sequence of S. pyogenes Cas9 protein may be found in the SwissProt database under accession number Q99ZW2. The Cas9 nuclease may be a Cas9 hotnolog or ortholog. Mutant Cas9 nucleases that exhibit improved specificity may also be used (see, e.g., Ann Ran et al. Cell 154(6) 1380-89 (2013), which is herein incorporated by reference in its entirety for all purposes and particularly for all teachings relating to mutant Cas9 nucleases with improved specificity for target nucleic acids). The nucleic acid manipulation reagents can also include a deactivated Cas9 nucleases (dCas9). Deactivated Cas9 binding to nucleic acid elements alone may repress transcription by stericany hindering RNA polymerase machinery. Further, deactivated Cas may be used as a homing device for other proteins e.g., transcriptional repressor, activators and recruitment domains) that affect gene expression at the target site without introducing irreversible mutations to the target nucleic acid. For example, dCas9 can be fused to transcription repressor domains such as KRAB or SID effectors to promote epigenetic silencing at a target site. Cas9 can also be converted into a synthetic transcriptional activator by fusion to VP16/VP64 or p64 activation domains. In some instances, a mutant Type II nuclease, referred to as enhanced Cas9 (eCa9) nuclease, is used in place of the wild-type Cas9 nuclease. The enhanced Cas9 has been rationally engineered to improve specificity by weakening non-target binding. This has been achieved by neutralizing positively charged residues within the non-target strand groove (Slaymaker et al., 2016).

In some embodiments, the Cas9 nucleases direct cleavage of one or both strands at the location of a target sequence, such as within the target sequence and/or within the complement of the target sequence. In some embodiments, the Cas9 nucleases directs cleavage of one or both strands within about 1, 2, 3, 4, 5, 6, 7, 8, 9, 10, 15, 20, 25, 50, 100, 200, 500, or more base pairs from the first or last nucleotide of a target sequence.

Following directed DNA cleavage by the Cas9 nuclease, there are two modes of DNA repair available to the cell: homology directed repair (HDR) and non-homologous end joining (NHEJ). While seamless correction of the mutation by HDR following Cas9 cleavage close to the mutation site is attractive, the efficiency of this method means that it could only be used for in vitro/ex vivo modification of stem cells or induced pluripotent stem cells (iPSC) with an additional step to select those cells in which repair had taken place and purify those modified cells only. HDR does not occur at a high frequency in cells. Fortunately NHEJ occurs at a much higher efficiency and may be suitable for the dominant-negative mutations described for many of the conical dystrophies. In additional embodiments, the Cas9 nuclease is from Streptococcus. In yet additional embodiments, the Cas9 nuclease is from Streptococcus pyogenes, Streptococcus dysgalactiae, Streptococcus canis, Streptococcus equi, Streptococcus iniae, Streptococcus phocae, Streptococcus pseudoporcinus, Streptococcus oralis, Streptococcus pseudoporcinus, Streptococcus infantarius Streptococcus mutatis, Streptococcus agatactiae, Streptococcus caballi, Streptococcus equunus, Streptococcus sp. oral taxon, Streptococcus mitis, Streptococcus gallolyticus, Streptococcus gordonii, or Streptococcus pasteurianus, or variants thereof. Such variants may include D10A Nickasc, Spy Cas9-HF1 as described in Klemsliver et al, 2016 Nature, 529, 490-495, or Spy eCas9 as described in Slaymaker et al., 2016 Science, 351(6268), 84-88. In additional embodiments, the Cas9 nuclease is from Staphylococcus. In yet additional embodiments, the Cas9 nuclease is from Staphylococcus aureus, S. simiae, S. auricularis, S. carnosus, S. condimenti, S. massiliensis, S. piscifermentans, S. simulans, S. capitis, S. caprae, S. epidermidis, S. saccharolyticus, S. devriesei, S. haemolyticus, S. hominis, S. agnetis, S. chromogenes, S. felis, S. delphini, S. hyicus, S. intermedius, S. lutrae, S. microti, S. muscae, S. pseudintermedius, S. rostri, S. schleiferi, S. lugdunensis, S. arlettae, S. cohnii, S. equorum, S. gallinarum, S. kloosii, S. leei, S. nepalensis, S. saprophyticus, S. succinus, S. xylosus, S. fleurettii, S. lentus, S. sciuri, S. stepanovicii, S. vitulinus, S. simulans, S. pasteuri, S. warneri, or variants thereof.

In further embodiments, the Cas9 nuclease excludes Cas9 nuclease from Streptococcus pyogenes.

In additional embodiments, the Cas9 nuclease comprises an amino acid sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93,94, 95, 96, 97, 98, 99 or 100% sequence identity with an amino acid sequence selected from the group consisting of SEQ ID NO: 4 or 8. In yet further embodiments, the nucleotide molecule encoding Cas9 nuclease comprises a nucleotide sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identify with a nucleotide sequence selected from the group consisting of SEQ ID NO: 3 or 7.

In some embodiments, the Cas9 nuclcaseis an enhanced Cas9 nuclease that has one or more mutations improving specificity of the Cas9 nuclease. In additional embodiments, the enhanced Cas9 nuclease is from a Cas9 nuclease from Streptococcus pyogenes having one or more mutations neutralizing a positively charged groove, positioned between the HNH, RuvC, and PAM-interacting domains in the Cas9 nuclease. In yet additional embodiments, the Cas9 nuclease comprises an amino acid sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a mutant amino acid sequence of a Cas9 nuclease from Streptococcus pyogenes (e.g., SEQ ID NO 4) with one or more mutations selected from the group consisting of (i) K855A, (ii) K810A, K1003A and R1060A, and (iii) K848A, K1003A and R1060A. In yet further embodiments, the nucleotide molecule encoding Cas9 nuclease comprises a nucleotide sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a nucleotide sequence encoding the mutant amino acid sequence.

In some embodiments, the CRISPR/Cas9 system and the methods using the CRISPR/Cas9 system described herein alter a DNA sequence by the NHEJ. In additional embodiments, the CRISPR/Cas9 system or the vector described herein does not include a repair nucleotide molecule.

In some embodiments, the methods described herein alter a DNA sequence by the HDR, for example, as shown in FIG. 14. In additional embodiments, this HDR approach could be used in an ex vivo approach to gene therapy in MECD. In further embodiments, this approach may not be allele specific and may be used to repair mutations in KRT12 codons 129, 130, 132, 133 and 135.

In some embodiments, the CRISPR/Cas9 system or the vector described herein may further comprise a repair nucleotide molecule. The target polynucleotide cleaved by the Cas9 nuclease may be repaired by homologous recombination with the repair nucleotide molecule, which is an exogenous template polynucleotide. This repair may result in a mutation comprising an insertion, deletion, or substitution of one or more nucleotides of said target polynucleotide. The repair nucleotide molecule introduces a specific allele (e.g., a wild-type allele) into the genome of one or more cells of the plurality of stem cells upon repair of a Type II nuclease induced DSB through the HDR pathway. In some embodiments, the repair nucleotide molecule is a single stranded DNA (ssDNA). In other embodiments, the repair nucleotide molecule is introduced into the cell as a plasmid vector. In some embodiments, the repair nucleotide molecule is 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 105, 105 to 110, 110 to 115, 115 to 120, 120 to 125, 125 to 130, 130 to 135, 135 to 140, 140 to 145, 145 to 150, 150 to 155, 155 to 160, 160 to 165, 165 to 170, 170 to 175, 175 to 180, 180 to 185, 185 to 190, 190 to 195, or 195 to 200 nucleotides in length. In some embodiments, the repair nucleotide molecule is 200 to 300, 300, to 400, 400 to 500, 500 to 600, 600 to 700, 700 to 800, 800 to 900, 900 to 1,000 nucleotides in length. In other embodiments, the repair nucleotide molecule is 1,000 to 2,000, 2,000 to 3,000, 3,000 to 4,000, 4,000 to 5,000, 5,000 to 6,000, 6,000 to 7,000, 7,000 to 8,000, 8,000 to 9,000, or 9,000 to 10,000 nucleotides in length. In some embodiments, the repair nucleotide molecule is capable of undergoing homologous recombination by the HDR pathway at a region of a stem cell genome that includes a mutation associated with a corneal dystrophy as described herein (i.e., a “corneal dystrophy target nucleic acid”). In certain embodiments, the repair nucleic acid is able to homologously recombine with a. target nucleic acid within the TGFBI, KRT12, GSN, and UBIAD1 gene. In particular embodiments, the repair nucleotide molecule is able to homologously recombine with a nucleic acid in the KRT12 gene encoding a mutant amino acid described herein (e.g., Leu132Pro). In some embodiments, the vector includes multiple repair nucleotide molecules.

The repair nucleotide molecule may further include a label for identification and sorting of cells described herein containing the specific mutation. Exemplary labels that can be included with the repair nucleotide molecule include fluorescent labels and nucleic acid barcodes that are identifiable by length or sequence.

In additional embodiments, the CRISPR/Cas9 system or the vector described herein may include at least one nuclear localization signal (NLS). In additional embodiments, the sgRNA and the Cas9 nuclease are included on the same vector or on different vectors.

In another aspect, the present disclosure is related to methods of altering expression of at least one gene product comprising introducing the engineered CRISPR/Cas9 system described herein into a cell containing and expressing a DNA molecule having a target sequence and encoding the gene product. The engineered CRISPR/Cas9 system can be introduced into cells using any suitable method. In some embodiments, the introducing may comprise administering the engineered CRISPR/Cas9 system described herein to cells in culture, or in a host organism.

Exemplary methods for introducing the engineered CRISPR/Cas9 system include, but are not limited to, transfection, electroporation and viral-based methods. In some cases, the one or more cell uptake reagents are transfection reagents. Transfection reagents include, for example, polymer based (e.g., DEAE dextran) transfection reagents and cationic liposome-mediated transfection reagents. Electroporation methods may also be used to facilitate uptake of the nucleic acid manipulation reagents. By applying an external field, an altered transmembrane potential in a cell is induced, and when the transmembrane potential net value (the sum of the applied and the resting potential difference) is larger than a threshold, transient permeation structures are generated in the membrane and electroporation is achieved. See, e.g., Gehl et al., Acta Physiol. Scand. 177:437-447 (2003). The engineered CRISPR/Cas9 system also be delivered through viral transduction into the cells. Suitable viral delivery systems include, but are not limited to, adeno-associated virus (AAV), retroviral and lentivirus delivery systems. Such viral delivery systems are useful in instances where the cell is resistant to transfection. Methods that use a viral-mediated delivery system may further include a step of preparing viral vectors encoding the nucleic acid manipulation reagents and packaging of the vectors into viral particles. Other method of delivery of nucleic acid reagents include, but are not limited to, lipofection, nucleofection, microinjection, biolistics, virosomes, liposomes, immunoliposomes, polycation or lipid:nucleic acid conjugates, naked DNA, artificial virions, and agent-enhanced uptake of nucleic acids. See, also Neiwoehner et al., Nucleic Acids Res. 42:1341-1353 (2014), and U.S. Pat. Nos. 5,049,386, 4,946,787; and 4,897,355, which are herein incorporated by reference in its entirety for all purposes, and particularly for all teachings relating to reagent delivery systems. In some embodiments, the introduction is performed by non-viral vector delivery systems include DNA plasmids, RNA (e.g., a transcript of a vector described herein), naked nucleic acid, and nucleic acid complexed with a delivery vehicle, such as a liposome. Delivery can be to cells (e.g., in vitro or ex vivo administration) or target tissues (e.g., in vivo administration).

The cells that have undergone a nucleic acid alteration event (i.e., a “altered” cell) can be isolated using any suitable method. In some embodiments, the repair nucleotide molecule further comprises a nucleic acid encoding a selectable marker. In these embodiments, successful homologous recombination of the repair nucleotide molecule a host stem cell genome is also accompanied by integration of the selectable marker. Thus, in such embodiments, the positive marker is used to select for altered cells. In some embodiments, the selectable marker allows the altered cell to survive in the presence of a drug that otherwise would kill the cell. Such selectable markers include, but are not limited to, positive selectable markers that confer resistance to neomycin, puromycin or hygromycin B. In addition, a selectable marker can be a product that allows an altered cell to be identified visually among a population of cells of the same type, some of which do not contain the selectable marker. Examples of such selectable markers include, but are not limited to the green fluorescent protein (GFP), which can be visualized by its fluorescence; the luciferase gene, which, when exposed to its substrate luciferin, can be visualized by its luminescence; and β-galactosidase (β-gal), which, when contacted with its substrate, produces a characteristic color. Such selectable markers are well known in the art and the nucleic acid sequences encoding these markers are commercially available (see, e.g., Sambrook et al., Molecular Cloning: A Laboratory Manual (Cold Spring Harbor Laboratory Press 1989). Methods that employ selectable markers that can be visualized by fluorescence may further be sorted using Fluorescence Activated Cell Sorting (FACS) techniques. Isolated manipulated cells may be used to establish cell lines for transplantation. The isolated altered cells can be cultured using any suitable method to produce a stable cell line.

In some embodiments, the engineered CRISPR/Cas9 system comprises (a) a first regulatory element operably linked to the sgRNA that hybridizes with the target sequence described herein, and (b) a second regulatory element operably linked to the nucleotide molecule encoding Cas9 nuclease, wherein components (a) and (b) are located on a same vector or different vectors of the system, the sgRNA targets the target sequence, and the Cas9 nuclease cleaves the DNA molecule. The target sequence may be a nucleotide sequence complementary to from 16 to 25 nucleotides adjacent to the 5′ end of a PAM. Being “adjacent” herein means being within 2 or 3 nucleotides of the site of reference, including being “immediately adjacent,” which means that there is no intervening nucleotides between the immediately adjacent nucleotide sequences and the immediate adjacent nucleotide sequences are within 1 nucleotide of each other. In additional embodiments, the cell is a eukaryotic cell, or a mammalian or human cell, and the regulatory elements are eukaryotic regulators. In further embodiments, the cell is a stem cell described herein. In some embodiments, the Cas9 nuclease is codon-optimized for expression in a eukaryotic cell.

In some embodiments, the first regulatory element is a polymerase III promoter. In some embodiments, the second regulatory element is a polymerase II promoter. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol III promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retro-viral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the Cas9 nuclease provided herein may be an inducible the Cas9 nuclease that is optimized for expression in a temporal or cell-type dependent manner. The first regulatory element may be an inducible promoter that can be linked to the Cas9 nuclease include, but are not limited to tetracycline-inducible promoters, metallothionein promoters; tetracycline-inducible promoters, inethionine-inducible promoters (e.g., MET25, MET3 promoters); and galactose-inducible promoters (GAL1, GAL7 and GAL10 promoters). Other suitable promoters include the ADH1 and ADH2 alcohol dehydrogenase promoters (repressed in glucose, induced when glucose is exhausted and ethanol is made), the CUP1 metallothionein promoter (induced in the presence of Cu²⁺, Zn²⁺), the PHO5 promoter, the CYC1 promoter, the HIS3 promoter, the PGK promoter, the GAPDH promoter, the ADC1 promoter, the TRP1 promoter, the URA3 promoter, the LEU2 promoter, the ENO promoter, the TP1 promoter, and the AOX1 promoter.

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.

The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells.

In another aspect, the present disclosure is related to methods of preventing, ameliorating, or treating a disease associated with a gene mutation or single-nucleotide polymorphism (SNP) in a subject comprising altering expression of the gene product of the subject by the methods described above, wherein the DNA molecule comprises a mutant or SNP mutant sequence.

In another aspect, the present disclosure is related to methods of preventing, ameliorating, or treating corneal dystrophy associated with a gene mutation or SNP in a subject. Subjects that can be treated with the methods include, but are not limited to, mammalian subjects such as a mouse, rat, dog, baboon, pig or human. In some embodiments, the subject is a human. The methods can be used to treat subjects at least 1 year, 2 years, 3 years, 5 years, 10 years, 15 years, 20 years, 25 years, 30 years, 35 years, 40 years, 45 years, 50 years, 55 years, 60 years, 65 years, 70 years, 75 years, 80 years, 85 years, 90 years, 95 years or 100 years of age. In some embodiments, the subject is treated for at least one, two, three, or four corneal dystrophies. For example, a single or multiple crRNA or sgRNA may be designed to alter nucleotides at a plurality of mutant or SNP sites associated with a single or multiple corneal dystrophies or at ancestral mutation or SNP sites.

As used herein, a “corneal dystrophy” refers to any one of a group of hereditary disorders in the outer layer of the eye (cornea). For example, the conical dystrophy may be characterized by bilateral abnormal deposition of substances in the cornea. Corneal dystrophies include, but are not limited to the following four IC3D categories of corneal dystrophies (see, e.g., Weiss et al., Cornea 34(2): 117-59 (2015)): epithelial and sub-epithelial dystrophies. epithelial-stromal TGFβI dystrophies, stromal dystrophies and endothelial dystrophies. In some embodiments, the corneal dystrophy is selected from the group consisting of Epithelial basement membrane dystrophy (EBMD), Meesmann corneal dystrophy (MECD), Thiel-Behnke corneal dystrophy (TBCD), Lattice corneal dystrophy (LCD), Granular corneal dystrophy (GCD), and Schnyder corneal dystrophy (SCD). In additional embodiments, the corneal dystrophy herein excludes MECD.

In additional embodiments, the corneal dystrophy is caused by one or more mutations, including SNP, is located in a gene selected from the group consisting of Transforming growth factor, beta-induced (TGFBI), keratin 3 (KRT3), keratin 12 (KRT12), GSN, and UbiA prenyltransfera.se domain containing 1 (UBIAD1). In further embodiments, the mutation or SNP site results in encoding a mutant amino acid in a mutant protein as shown herein. In further embodiments, a mutant sequence comprising the mutation or SNP site encodes a mutant protein selected from the group consisting of (i) mutant TGFBI proteins comprising a mutation corresponding to Leu509Arg, Arg666Ser, Gly623Asp, Arg555Gln, Arg124Cys, Val505Asp, Ile522Asn, Leu569Arg, His572Arg, Arg496Trp, Pro501Thr, Arg514Pro, Phe515Leu, Leu518Pro, Leu518Arg, Leu527Arg, Thr538Pro, Thr538Arg, Val539Asp, Phe540del, Phe540Ser, Asn544Ser, Ala546Thr, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Valb 624_Val625del, Val624Met, Val625Asp, His626Arg, His626Pro, Val627SerfsX44, Thr629_Asn630insAsnValPro, Val631AspArg666Ser, Arg555Trp, Arg124Ser, Asp123delins, Arg124His, Arg124Leu, Leu509Pro, Leu103_Ser104del, Val113Ile, Asp123His, Arg124Leu, and/or Thr125_Glu126del in TGFBI, for example, of Protein Accession No. Q15582; (ii) mutant KRT3 proteins comprising a mutation corresponding to Glu498Val, Arg503Pro, and/or Glu509Lys in Keratin 3 protein, for example, of Protein Accession No. P12035 or NP_476429.2; (iii) mutant KRT12 proteins with Met129Thr, Met129Val, Gln130Pro, Leu132Pro, Leu132Va, Leu132His, Asn133Lys, Arg135Gly, Arg135Ile, Arg135Thr, Arg135Ser, Ala137Pro, Leu140Arg, Val143Leu, Val143Leu, Lle391_Leu399dup, Ile 426Val, Ile 426Ser, Tyr429Asp, Tyr429Cys, Arg430Pro, and/or Leu433Arg in KRT12, for example, of Protein Accession No. Q99456.1 or NP_900214.1; (iv) mutant GSN proteins with Asp214Tyr in GSN, for example, of Protein Accession No. P06396; and (v) mutant UBIAD1 proteins comprising a mutation corresponding to Ala97Thr, Gly98Ser, Asn102Ser, Asp112Asn, Asp112Gly, Asp118Gly, Arg119Gly, Leu121Val, Leu121Phe, Val122Glu, Val122Gly, Ser171Pro, Tyr174Cys, Thr175Ile, Glyl77Arg, Lys181Arg, Gly186Arg, Leu188His, Asn232Ser, Asn233His, Asp236Glu, and/or Asp240Asn in UBIAD1, for example, of Protein Accession No. Q9Y5Z9. For example, a mutant sequence comprising the mutation or SNP site encodes at least a part of mutant TGFBI protein mutated by replacing Lett with Arg at amino acid position corresponding the amino acid position 509 of Protein Accession No, Q15582. In this case, a mutation at the mutation or SNP site may be responsible for encoding the mutant amino acid at amino acid position corresponding the amino acid position 509 of Protein Accession No. Q15582. As used herein, a mutation “corresponding to” a particular mutation in a human protein may include a mutation in a different species that occur at the corresponding site of the particular mutation of the human protein. Also as used herein, when a mutant protein is described to include a particular mutant, for example, of Leu509Arg, such a mutant protein may comprise any mutation that occurs at a mutant site corresponding to the particular mutant in a relevant human protein, for example, in TG-FBI protein of Protein Accession No. Q15582 as described herein.

In some embodiments, the mutant described herein excludes any mutant in KRT12 protein, In some embodiments, the mutant described herein excludes a. mutation corresponding to Leu132Pro in KRT12, for example, of Protein Accession No. Q99456.1. In further embodiments, the mutatoin or SNP described herein excludes any SNP that occurs in KRT12 gene. In yet further embodiments, the mutation or SNP described herein excludes any SNP that results in the Leu132Pro mutation in KRT12 protein. The mutatoin or SNP may further exclude the SNP at a PAM site (AAG>AGG) that results in the Leu132Pro mutation in KRT12 protein.

In some embodiments, the CRISPR/Cas9 system and the methods using the same described. herein may alter mutant sequences at a plurality of SNP sites or ancestral SNPs. Such methods would utilize flanking PAMs as shown in FIGS. 15-16. In additional embodiments, the mutant sequence described herein may comprise at least one, two, three, four or more SNP sites, and the method described herein alters the expression of the gene product related to at least one, two, three, four or more of the SNP sites. For example, the method described herein may alter the expression of mutant TGFBI proteins at both R514P and L518R, or KRT12 proteins at both R135T and L132P. In some embodiments, sgRNA may comprise a target sequence adjacent to a PAM site located in the flanking intron that is common to both wild-type and mutant alleles in tandem with a sgRNA adjacent to a PAM site that is specific to the mutant allele.

The human genome is diploid by nature; every chromosome with the exception of the X and Y chromosomes in males is inherited as a pair, one from the male and one from the female. When seeking stretches of contiguous DNA sequence larger than a few thousand base pairs, a determination of inheritance is crucial to understand from which parent these blocks of DNA originate. Furthermore, most SNPs exist within the human genome as heterozygous, i.e. inherited either from the male or the female. Longer read sequencing technologies have been utilized in attempts to produce a haplotype-resolved genome sequences, i.e. haplotype phasing. Thus, when investigating the genomic sequence of a particular stretch of DNA longer than 50 kbs, a haplotype phased sequence analysis may be utilized to determine which of the paired chromosomes carries the sequence of interest. Longer phased sequencing reads may be employed to determine whether the SNP of interest would be suitable as a target fbr the CRISPR/Cas9 gene editing system described herein.

In one aspect, the methods described herein comprise identifying targetable mutations or SNPs on either side of disease-causing mutation or SNP are identified to silence the disease-causing mutation or SNP. In some embodiments, a block of DNA is identified in a phased sequencing experiment. In some embodiments, the mutation or SNP of interest is not a suitable substrate for the CRISPR/Cas9 system, and identifying mutations or SNPs on both side of the disease-causing mutations or SNP that are suitable for CRISPR/Cas9 cleavage allows removing a segment of DNA that includes the disease-causing mutations or SNP. In some embodiments, the read length may be increased so as to gain longer contiguous reads and a haplotype phased genome by using a technology described in Weisenfeld N I, Kumar V, Shah P, Church D M, Jaffe D B. Direct determination of diploid genome sequences. Genome research. 2017; 27(5):757-767, which is herein incorporated by reference in its entirety

In some embodiments of the methods provided herein, therapy is used to provide a positive therapeutic response with respect to a disease or condition (e.g., a corneal dystrophy). By “positive therapeutic response” is intended an improvement in the disease or condition, and/or an improvement in the symptoms associated with the disease or condition. The therapeutic effects of the subject methods of treatment can be assessed using any suitable method. In some embodiments, in case of the corneal dystrophy that involves protein deposition on the cornea, treatment is assessed by the reduction of protein deposition on the cornea of the subject after treatment as compare to a control (e.g., the amount of protein deposition prior to treatment). In certain embodiments, the subject methods reduce the amount of corneal protein deposition in the subject by at least 5%, 10%, 15%, 20%, 25%, 30%, 35%, 40%, 45%, 50%, 55%, 60%, 65%, 70%, 75%, 80%, 85%, 90%, 95%, or 99% as compared to the cornea prior to undergoing treatment. Corneal opacity can also be used to assess the therapeutic effect using the subject methods. Further, in some embodiments, treatment is assessed by visual function. Assessment of visual function in the subject can be carried out using any suitable test known in the art including, but not limited to, assessments of uncorrected visual acuity (UCVA), best-corrected visual acuity (BCVA) and brightness acuity test (BAT). See, e.g., Awaad et al., Am J Ophthalmol. 145(4): 656-661 (2008) and Sharhan et al., Br J Ophthalmol 84:837-841 (2000), which are incorporated by reference in their entirety for all purposes, and particularly for all teachings relating to standards for assessing visual acuity. In certain embodiments, the subject's visual acuity improves by at least 5%, 10%, 15%, 20%, 25%, 30%, 35%, 40%, 45%, 50%, 55%, 60%, 65%, 70%, 75%, 80%, 85%, 90%, 95%, or 99% as compared prior to undergoing treatment.

In some embodiments, methods of preventing, ameliorating, or treating corneal dystrophy associated with SNP in a subject may comprise administering to the subject an effective amount of the engineered CRISPR/Cas9 system described herein. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The engineered CRISPR/Cas9 system described herein may comprise at least one vector comprising (i) a nucleotide molecule encoding Cas9 nuclease described herein, and (ii) sgRNA described herein. The sgRNA may comprise a target sequence adjacent to the 5′-end of a protospacer adjacent motif (PAM), and/or hybridize to a first target sequence complementary to a second target sequence adjacent to the 5′ end of the PAM. In some embodiments, the target sequence or the PAM comprises the SNP site. In some embodiments, the Cas9 nuclease and the sgRNA do not naturally occur together. In some embodiments, DNA cleavage by the Cas9 nuclease requires, in addition to sequence-specific annealing between the guide RNA molecule and the target DNA, the presence of a protospacer adjacent motif (PAM), which lies immediately 3′ of the guide RNA binding site. The sequence of this PAM site is specific to the Cas9 nuclease being used. In additional embodiments, the PAM comprises the SNP site. In yet additional embodiments, the PAM consists of a PAM selected from the group consisting of NGG and NNGRRT, wherein N is any of A, T, G, and C, and R is A or G. In further embodiments, the administering comprises introducing the engineered CRISPR/Cas9 system into a cornea (e.g., corneal stroma) of the subject, for example, by injecting the engineered CRISPR/Cas9 system into a cornea (e.g., corneal stroma) of the subject and/or by introducing the engineered CRISPR/Cas9 system into a cell containing and expressing a DNA molecule having the target sequence.

In another aspect, the present disclosure is related to methods of treating corneal dystrophy in a subject in need thereof, comprising:(a) obtaining a plurality of stem cells comprising a nucleic acid mutation in a corneal dystrophy target nucleic acid from the subject; (b) manipulating the nucleic acid mutation in one or more stem cells of the plurality of stem cells to correct the nucleic acid mutation, thereby forming one or more manipulated stem cells; (c) isolating the one or more manipulated stem cells; and (d) transplanting the one or more manipulated stem cells into the subject, wherein manipulating the nucleic acid mutation in the one or more stem cells of the plurality of stem cells includes performing any of the methods of altering expression of a gene product or of preventing, ameliorating, or treating a disease associated with SNP in a subject as described herein.

The subject methods may include obtaining a plurality of stem cells. Any suitable stem cells can be used for the subject method, depending on the type of corneal dystrophy to be treated. In certain embodiments, the stem cell is obtained from a heterologous donor. In such embodiments, the stem cells of the heterologous donor and the subject to be treated are donor-recipient histocompatible. In certain embodiments, autologous stem cells are obtained from the subject in need of the treatment for corneal dystrophy. Obtained stem cells carry a mutation in a gene associated with the particular corneal dystrophy to be treated (e.g., stem cells having a mutation in a TGFBI of a subject having an epithelial-stromal dystrophy, as discussed above). Suitable stem cells include, but are not limited to, dental pulp stem cells, hair follicle stem cells, mesenchymal stem cells, umbilical cord lining stem cells, embryonic stem cells, oral mucosal epithelial stem cells and limbal epithelial stem cells.

In some embodiments, the plurality of stem cells includes limbal epithelial stem cells. Limbal epithelial stem cells (LESCs) are located in the limbal region of the cornea and are responsible for the maintenance and repair of the corneal surface. Without being bound by any particular theory of operation, it is believed that LESCs undergo asymmetric cell division producing a stem cell that remains in the stem cell niche to repopulate the stem cell pool, and a daughter early transient amplifying cell (eTAC). This more differentiated eTAC is removed from the stem cell niche and is able to divide to further produce transient amplifying cells (TAC), eventually giving rise to terminally differentiated cells (DC). LESCs can be obtained, for example, by taking a biopsy from the subject's eye. See, e.g., Pellegrini et al., Lancet 349: 990-993 (1997). LESCs obtained from linibal biopsies can be isolated and sorted for use in the subject methods using any suitable technique including, but not limited to, fluorescence activated cell sorting (FACS) and centrifugation techniques. LESCs can be sorted from biopsies using positive expression of stem cell associated markers and negative expression of differentiation markers. Positive stem cell markers include, but are not limited to, transcription factor p63, ABCG2, C/EBPδ and Bmi-1. Negative corneal specific markers include, but are not limited to, cytokeratin 3 (CK3), cytokeratin 12 (CK12), connexin 43, and involucrin. In some embodiments, the plurality of stem cells is positive for expression of p63, ABCG2 or combinations thereof. In certain embodiments, at least 65%, 70%, 75%, 80%, 85%, 86%. 88%, 89%, 90%, 91%, 92%, 93%, 94%, 95%, 96%, 97%, 98% or 99% of the cells in the plurality of stem cells express p63, ABCG2, C/EBPδ and Bmi-1 or combinations thereof. In some embodiments, the plurality of stem cells is negative for expression of CK3, CK12, connexin 43, involucrin or combinations thereof. In certain embodiments, at least 65%, 70%, 75%, 80%, 85%, 86%. 88%. 89%, 90%, 91%, 92%, 93%, 94%, 95%, 96%, 97%, 98% or 99% of the cells in the plurality of stem cells do not express CK12, connexin 43, involucrin or combinations thereof. Other markers useful for LESC are described, for example, in Takacs et al., Cytometry A 75: 54-66 (2009), Which is incorporated by reference in its entirety fbr all purposes, and particularly for all teachings relating to LESC markers. Stem cell features such as cell size and high nuclear to cytoplasmic ratio can also be used to aid in the identification of LESCs.

In addition to LESCs, other stem cells isolated from the subject's cornea can also be used with the subject methods. Exemplary corneal stem cells include, but are not limited to, stromal stem cells, stromal fibroblast-like cells, stromal mesenchymal cells, neural crest derived corneal stem cells, and putative endothelial stem cells.

In some embodiments, the cells used with the subject methods are stromal stem cells isolated from the subject's cornea. Stromal stem cells can be isolated using any suitable method including, but not limited to, those described in Funderburgh et al., FASEB J 19: 1371-1373 (2005); Yoshida et al., Invest Ophtalmol Vis Sci 46: 1653-1658 (2005); Du et al. Stem Cells 1266-1275 (2005); Dravida et al., Brain Res Dev Brain Res 160:239-251 (2005); and Polisetty et al. Mol Vis 14: 431-442 (2008), which are incorporated by reference in their entirety for all purposes, and particularly for all teaching relating to the isolation and culturing of various stromal stem cells.

Markers that are characteristic of these stromal stem cells include, but are not limited to, Bmi-1, Kit, Notch-1, Six2, Pax6, ABCG2, Spag10, and p62/OSIL. In some embodiments, at least 65%, 70%, 75%, 80%. 85%, 86%. 88%, 89%, 90%, 91%, 92%, 93%, 94%, 95%, 96%, 97%, 98% or 99% of cells in the plurality of stem cells express Bmi-1, Kit, Notch-1, Six2, Pax6, ABCG2, Spag10, or p62/OSIL or combinations thereof. In certain embodiments, the stromal stem cells are positive for CD31, SSEA-4, CD73, CD105 and negative for CD34, CD45, CD123, CD133, CD14, CD106 and HLA-DR: In certain embodiments, at least 65%, 70%, 75%, 80%. 85%, 86%, 88%. 89%, 90%, 91%, 92%, 93%, 94%, 95%, 96%, 97%, 98% or 99% of cells in the plurality of stem cells are positive for CD31, SSEA-4, CD73, CD105 and negative for CD34, CD45, CD123, CD133, CD14, CD106 and HLA-DR. In yet other embodiments, the stromal step cells are positive for CD105, CD106, CD54, CD166, CD90, CD29, CD71, Pax6 and negative for SSEA-1, Tral-81, Tral-61, CD31, CD45, CD11a, CD11c, CD14, CD138, Flk1, Flt1, and VE-cadherin. In certain embodiments, at least 65%, 70%, 75%, 80%. 85%, 86%. 88%. 89%, 90%, 91%, 92%, 93%, 94%, 95%, 96%, 97%, 98% or 99% of cells in the plurality of stem cells are positive for CD105, CD106, CD54, CD166, CD90, CD29, CD71, Pax6 and negative for SSEA-1, Tral-81, Tral-61, CD31, CD45, CD11a, CD11c, CD14, CD138, Flk1, Flt1, and VE-cadherin.

In certain embodiments, the cells used with the subject methods are endothelial stem cells isolated from the subject's cornea. Methods of isolating such stem cells are described, for instance, in Engelmann et al., Invest Ophthalmol Vis Sci 29: 1656-1662 (1988), which is incorporated by reference in their entirety for all purposes, and particularly for all teachings relating to the isolating and culturing of corneal endothelial stem cells.

After isolation, the plurality of stem cells (e.g., LESCs) can be cultured using any suitable method to produce a stable cell line. For instance, cultures can be maintained in the presence or absence of fibroblast cells (e.g., 3T3) as feeder cells. In other instances, human amniotic epithelial cells or human embryonic fibroblasts are used as feeder layers for cultures. Suitable techniques for the culturing of LESCs are further described in Takacs et al. Cytometry A 75: 54-66 (2009), Shortt et al., Surv Opthalmol Vis Sci 52: 483-502 (2007), and Cauchi et al. Am J Ophthalmol 146: 251-259 (2008), which are incorporated by reference in their entirety for all purposes, and particularly for all teachings relating to the culturing of LESCs.

After the isolation of the plurality stem cells, the nucleic acid mutation in one or more stem cells in the plurality of stem cells is manipulated or altered by the methods described herein to correct a nucleic acid mutation in a corneal dystrophy target nucleic acid. As used herein, a “corneal dystrophy target nucleic acid” refers to a nucleic acid that includes a mutation associated with one or more of the corneal dystrophies described herein.

Stem cells to be manipulated include individual isolated stem cells or stem cells from a stem cell line established from the isolated stem cells. Any suitable genetic manipulation method may be used to correct the nucleic acid mutation in the stem cells.

In another aspect, provided herein are kits comprising the CRISPR/Cas9 system for the treatment of the corneal dystrophy. In some embodiments, the kit includes one or more sgRNAs described herein, a Cas9 nuclease and a repair nucleotide molecule that includes a wild-type allele of the mutation to be repaired as described herein. In some embodiments, the kit also includes agents that facilitate uptake of the nucleic acid manipulation by cells, for example, a transfection agent or an electroporation buffer. In some embodiments, the subject kits provided herein include one or more reagents for the detection or isolation of stem cells, for example, labeled antibodies for one or more positive stem cell markers that can be used in conjunction with FACS.

In another aspect, the present disclosure is related to an sgRNA pair, and a kit comprising the sgRNA pair comprising at least two sgRNAs for CRISPR/Cas9 system to silence a disease-causing mutation or SNP, for example, for preventing, ameliorating or treating corneal dystrophies. In some embodiments, the sgRNA pair is for silencing a disease-causing mutation or SNP in TGFBI gene. The sgRNA pair comprises an sgRNA comprising a guide sequence for PAM generating an ancestral mutation or SNP in TGFBI gene, for example, in an intron in cis with a disease-causing mutation or SNP. In additional embodiments, the sgRNA pair comprises an sgRNA comprising a common guide sequence for PAM generating an ancestral SNP in intronic regions of TGFBI gene.

In some embodiments, the present disclosure is related to an sgRNA pair designed for CRISPR/Cas9 system, the sgRNA pair comprising (i) a first sgRNA comprising (a) a first crRNA sequence for a first protospacer adjacent motif (PAM) generating mutation or single-nucleotide polymorphism (SNP) at 3′-end side of a disease-causing mutation or SNP in cis, and (b) a tracrRNA sequence, in which the first crRNA sequence and the tracrRNA sequence do not naturally occur togethe (ii) a second sgRNA comprising (a) a second crRNA guide sequence for a second PAM generating mutation or SNP at 5′-end side of the disease-causing mutation or SNP in cis; (b) a tracrRNA sequence, in which the second crRNA sequence and the tracrRNA sequence do not naturally occur together.

In additional embodiments, the CRISPR/Cas9 system is for preventing, ameliorating or treating corneal dystrophies. The PAM generating mutations or SNPs may be in TGFBI gene. In further embodiments, the PAM generating mutations or SNPs are in introns of TGFBI gene. In yet further embodiments, at least one of the first and second crRNA sequences comprises a nucleotide sequence selected from the group consisting of sequences listed in FIGS. 19-35; and/or at least one of the first and second crRNA sequences comprises a nucleotide sequence selected from the group consisting of sequences listed in Table 2.

### EXAMPLES

The following examples are presented to illustrate various embodiments of the invention. It is understood that such examples do not represent and are not intended to represent exclusive embodiments; such examples serve merely to illustrate the practice of this invention.

Mutation analysis: Mutations associated with various corneal dystrophies were analyzed to determine which were solely missense mutations or in-frame indels. This analysis indicates that fbr the majority of K12 and TGFBI disease, nonsense or frameshifiing indel mutations are not associated with disease. Furthermore, an analysis of the exome variant database confirmed that any naturally occurring nonsense, frameshifting indels or splice site mutations found in these genes are not reported to be associated with disease in these individuals.

Mutation analysis revealed that the following corneal-dystrophy genes are suitable for targeted nuclease gene therapy (Table 1).

TABLE 1
Genes and their associated corneal dystrophies
that are suitable for a CRISPR/Cas9 mediated approach.

| Gene | Associated Corneal Dystrophies |
|---|---|
| TGFBI | Avellino corneal dystrophy |
| | Reis-Bucklers corneal dystrophy |
| | Thiel-Behnke corneal dystrophy |
| | Grayson-Wilbrandt corneal dystrophy |
| | Lattice Corneal Dystrophy I & II |
| | Granular Corneal Dystrophy I, II & III |
| | Epithelial Basement Membrane Dystrophy |
| KRT3 | Meesemann Epithelial Corneal Dystrophy |
| KRT12 | Meesemann Epithelial Corneal Dystrophy |
| UBIAD1 | Schnyder corneal dystrophy |

An investigation of the suitable corneal dystrophy genes was conducted for this report to determine the number of mutations targetable by either a PAM-specific approach or a guide allele-specific approach. A PAM-specific approach requires the disease causing SNP to generate a novel PAM, whilst the allele specific approach involves the design of a guide containing the disease causing SNP. All non-disease causing SNPs in TGFBI that generate a novel PAM with a minor allele frequency (MAF) of >10% were identified and analyzed by the Benchlign's online genome-editing design tool. The selection of SNPs with a MAF of >10% may provide a reasonable chance that the SNP resulting in a. novel PAM will be found in cis with the disease causing mutation. Being “in cis” with the disease causing mutation refers to being on the same molecule of DNA or chromosome as the disease-causing mutation. The SNP resulting in a novel PAM may be found, for example, in intron or exon in TGFBI gene in cis with the disease-causing mutation. All variants within TGFBI were analyzed to determine whether a novel PAM was created (Table 2).

TABLE 2
The variants within TGFBI that result in a novel PAM that have a MAF of >10%. The novel PAM is shown with the required variant indicated in red.

| | | | Region Start | Region End | Variant | | | Novel PAM | | | | Population Genetics | | | | | |
|---|---|---|---|---|---|---|---|---|---|---|---|---|---|---|---|---|---|
| | | | Co-ordinate | Co-ordinate | Co-ordinate | | | (Required | Guide | | | All Indi- | | Ameri- | East | Euro- | South |
| Region | Number | Position | Chromosome 5 | Chromosome 5 | Chromosome 5 | SNP | Variant | variant in red) | Sequence | Strand | MAF | vidual | African | can | Asian | pean | Asian |
| Exon | 1 | | 136,028,895 | 136,029,189 | | | | | | | | | | | | | |
| Intron | 1 to 2 | Intronic | 136 029 | 136 033 | 136,032,206- | rs756462 | T/C | ccc | GAATCC | − | 0.31 | T: 69% | T: 56% | T: 63% | T: 64% | T: 79% | T: 85% |
| | | variant, | 190 | 762 | 136,032,306 | | | | ATGTAA | | | C: 31% | C: 44% | C: 37% | C: 36% | C: 21% | C: 15% |
| | | 1507bp away | | | | | | | GGATCT | | | | | | | | |
| | | from exon 2 | | | | | | | AG | | | | | | | | |
| Exon | 2 | | 136,033,763 | 136,033,861 | | | | | | | | | | | | | |
| Intron | 2 to 3 | Intronic | 136,033,862 | 136,044,057 | 136,042,063- | rs1989972 | A/C | atccca | CAGGGC | − | 0.43 | A: 43% | A: 26% | A: 51% | A: 42% | A: 52% | A: 49% |
| | | variant, | | | 136,042,163 | | | | TGTATT | | | C: 57% | C: 74% | C: 49% | C: 58% | C: 48% | C: 51% |
| | | 1945bp away | | | | | | | ACTGGG | | | | | | | | |
| | | from exon 3 | | | | | | | GC | | | | | | | | |
| | | Intronic | | | | rs1989972 | A/C | cca | CAGGGC | − | 0.43 | A: 43% | A: 26% | A: 51% | A: 42% | A: 52% | A: 49% |
| | | variant, | | | | | | | TGTATT | | | C: 57% | C: 74% | C: 49% | C: 58% | C: 48% | C: 51% |
| | | 1945bp away | | | | | | | ACTGGG | | | | | | | | |
| | | from exon 3 | | | | | | | GC | | | | | | | | |
| | | Intronic | | | | rs1989972 | N/C | ccc | AGGGCT | − | 0.43 | A: 43% | C: 26% | A: 51% | A: 42% | A: 52% | A: 49% |
| | | variant, | | | | | | | GTATTA | | | C: 57% | A: 74% | C: 49% | C: 58% | C: 48% | C: 51% |
| | | 1945bp away | | | | | | | CTGGGG | | | | | | | | |
| | | from exon 3 | | | | | | | CT | | | | | | | | |
| | | Intron | | | 136,043,042- | rs3805700 | A/G | agg | ATTCAT | + | 0.41 | A: 59% | A: 40% | A: 63% | A: 66% | A: 72% | A: 63% |
| | | variant, | | | 136,043,142 | | | | ATAGAA | | | G: 41% | G: 60% | G: 37% | G: 34% | G: 28% | G: 37% |
| | | 966bp away | | | | | | | GAAAG | | | | | | | | |
| | | from exon 3 | | | | | | | GAA | | | | | | | | |
| Exon | 3 | | 136,044,058 | 136,044,122 | | | | | | | | | | | | | |
| Intron | 3 to 4 | | 136,044,123 | 136,046,334 | | | | | | | | | | | | | |
| Exon | 4 | | 136,046,335 | 136,046,495 | | | | | | | | | | | | | |
| Intron | 4 to 5 | | 136,046,496 | 136,046,850 | | | | | | | | | | | | | |
| Exon | 5 | | 136,046,851 | 136,047,015 | | | | | | | | | | | | | |
| Intron | 5 to 6 | | 136,047,016 | 136,047,273 | | | | | | | | | | | | | |
| Exon | 6 | Synonymous | 136,047,274 | 136,047,420 | 136,047,250- | rs1442 | G/C | CCT | TTGCAT | − | 0.32 | C: 32% | C: 3% | C: 45% | C: 37% | C: 47% | C: 40% |
| | | variant, | | | 136,047,350 | | | | GGTGGT | | | G: 68% | G: 97% | G: 55% | G: 63% | G: 53% | G: 60% |
| | | protein | | | | | | | CGGCTT | | | | | | | | |
| | | position 217 | | | | | | | TC | | | | | | | | |
| Intron | 6 to 7 | Intronic | 136,047,421 | 136,049,438 | 136,047,489- | rs764567 | A/G | cgg | TCCTGT | + | 0.3 | A: 70% | A: 70% | A: 67% | A: 75% | A: 74% | A: 64% |
| | | variant, | | | 136,047,589 | | | | AGGGG | | | G: 30% | G: 30% | G: 33% | G: 25% | G: 26% | G36% |
| | | 119bp away | | | | | | | AACATA | | | | | | | | |
| | | from exon 6 | | | | | | | GAG | | | | | | | | |
| | | Intronic | | | | rs764567 | A/G | gcgggt | CTCCTG | + | 0.3 | A: 70% | A: 70% | A: 67% | A: 75% | A: 74% | A: 64% |
| | | variant, | | | | | | | TAGGGG | | | G: 30% | G: 30% | G: 33% | G: 25% | G: 26% | G36% |
| | | 119bp away | | | | | | | AACATA | | | | | | | | |
| | | from exon 6 | | | | | | | GA | | | | | | | | |
| | | Intronic | | | 136,047,638- | rs2073509 | A/G | agg | GTGTGT | + | 0.4 | T: 60% | T: 39% | T: 63% | T: 66% | T: 74% | T: 63% |
| | | variant, | | | 136,047,738 | | | | GGCTGC | | | G: 40% | G: 61% | G: 37% | G: 34% | G: 26% | G: 37% |
| | | 268bp away | | | | | | | AGCAGC | | | | | | | | |
| | | from variant | | | | | | | AC | | | | | | | | |
| | | Intronic | | | | rs2073509 | T/G | ggg | TGTGTG | + | 0.4 | T: 60% | T: 39% | T: 63% | T: 66% | T: 74% | T: 63% |
| | | variant, | | | | | | | GCTGCA | | | G: 40% | G: 61% | G: 37% | G: 34% | G: 26% | G: 37% |
| | | 268bp away | | | | | | | GCAGCA | | | | | | | | |
| | | from variant | | | | | | | CA | | | | | | | | |
| | | Intronic | | | | rs2073509 | T/G | cagggt | GGTGTG | + | 0.4 | T: 60% | T: 39% | T: 63% | T: 66% | T: 74% | T: 63% |
| | | variant, | | | | | | | TGGCTG | | | G: 40% | G: 61% | G: 37% | G: 34% | G: 26% | G: 37% |
| | | 268bp away | | | | | | | CAGCAG | | | | | | | | |
| | | from variant | | | | | | | CA | | | | | | | | |
| | | Intronic | | | 136,048,154- | rs2073511 | T/C | cct | GGAGA | − | 0.4 | T: 60% | T: 39% | T: 63% | T: 66% | T: 74% | T: 63% |
| | | variant, | | | 136,048,254 | | | | GGAGCT | | | G: 40% | G: 61% | G: 37% | C: 34% | C: 26% | C: 37% |
| | | 784bp away | | | | | | | TAGACA | | | | | | | | |
| | | from variant | | | | | | | GCG | | | | | | | | |
| | | Intronic | | | 136,048,704- | rs916951 | A/G | cgg | GTAATA | + | 0.37 | A: 63% | A: 95% | A: 53% | A: 58% | A: 49% | A: 50% |
| | | variant, | | | 136,048,804 | | | | GCAAAG | | (G) | G: 37% | G: 5% | G: 47% | G: 42% | G: 51% | G: 50% |
| | | 685bp from | | | | | | | GCTCAG | | | | | | | | |
| | | exon 7 | | | | | | | GG | | | | | | | | |
| Exon | 7 | | 136,049,439 | 136,049,580 | | | | | | | | | | | | | |
| Intron | 7 to 8 | Intronic | 136,049,581 | 136,052,906 | 136,050,039- | rs1137550 | T/C | actctg | ATCCGC | − | 0.37 | T: 63% | T: 52% | T: 64% | T: 66% | T: 74% | T: 63% |
| | | variant, | | | 136,050,139 | | | | CCACCT | | | C: 37% | C: 48% | C: 36% | C: 34% | C: 26% | C: 37% |
| | | 509bp from | | | | | | | TGTCCT | | | | | | | | |
| | | exon 7 | | | | | | | CC | | | | | | | | |
| Exon | 8 | Synonymous | 136,052,907 | 136,053,119 | 136,052,924- | rs1054124 | A/G | TGG | CATCGT | + | 0.39 | A: 61% | A: 46% | A: 64% | A: 66% | A: 74% | A: 63% |
| | | variant, | | | 136,053,024 | | | | TGCGGG | | | G: 39% | G: 54% | G: 36% | G: 34% | G: 26% | G: 37% |
| | | protein | | | | | | | GCTGTC | | | | | | | | |
| | | position 327 | | | | | | | TG | | | | | | | | |
| Intron | 8 to 9 | Intronic | 136,053,120 | 136,053,942 | 136,053,686- | rs6889640 | C/A | actctc | CCAGTC | − | 0.37 | A: 37% | A: 54% | A: 24% | A: 34% | A: 26% | A: 37% |
| | | variant, | | | 136,053,786 | | | | AGGAG | | | C: 63% | C: 46% | C: 76% | C: 66% | C: 74% | C: 63% |
| | | 207bp from | | | | | | | GAGAG | | | | | | | | |
| | | exon 9 | | | | | | | GGAG | | | | | | | | |
| Exon | 9 | | 136,053,943 | 136,054,080 | | | | | | | | | | | | | |
| Intron | 9 to 10 | | 136,054,081 | 136,054,715 | | | | | | | | | | | | | |
| Exon | 10 | | 136,054,716 | 136,054,861 | | | | | | | | | | | | | |
| Intron | 10 to 11 | Intronic | 136,054,862 | 136,055,679 | 136,055,587- | rs6860369 | A/G | ggg | CAAATC | + | 0.4 | A: 60% | A: 33% | A: 75% | A: 66% | A: 74% | A: 63% |
| | | variant, | | | 136,055,687 | | | | AGGAG | | | G: 40% | G: 67% | G: 25% | G: 34% | G: 26% | G: 37% |
| | | 43 bp from | | | | | | | GCCCCT | | | | | | | | |
| | | exon 11 | | | | | | | CGT | | | | | | | | |
| Exon | 11 | | 136,055,680 | 136,055,816 | | | | | | | | | | | | | |
| Intron | 11 to 12 | | 136,055,817 | 136,056,664 | | | | | | | | | | | | | |
| Exon | 12 | | 136,056,665 | 136,056,795 | | | | | | | | | | | | | |
| Intron | 12 to 13 | Intronic | 136,056,796 | 136,059,089 | 136,057,458- | rs6871571 | A/G | ttgaat | TGCAGC | + | 0.42 | A: 58% | A: 33% | A: 63% | A: 66% | A: 74% | A: 63% |
| | | variant, | | | 136,057,558 | | | | CTGTGT | | | G: 42% | G: 67% | G: 37% | G: 34% | G: 26% | G: 37% |
| | | 713bp from | | | | | | | TGGGAG | | | | | | | | |
| | | exon 12 | | | | | | | GA | | | | | | | | |
| Exon | 13 | | 136,059,090 | 136,059,214 | | | | | | | | | | | | | |
| Intron | 13 to 14 | Intronic | 136,059,215 | 136,060,833 | 136,059,694- | rs6893691 | A/G | cgg | AATCTC | + | 0.39 | A: 39% | A: 11% | A: 49% | A: 43% | A: 52% | A: 51% |
| | | variant, | | | 136,059,794 | | | | CCTGGC | | | G: 61% | G: 89% | G: 51% | G: 57% | G: 48% | G: 49% |
| | | 530bp from | | | | | | | TGCACC | | | | | | | | |
| | | exon 13 | | | | | | | TG | | | | | | | | |
| | | Intronic | | | 136,059,804- | rs1990199 | G/C | cca | TGCATA | − | 0.39 | C: 61% | C: 89% | C: 51% | C: 57% | C: 48% | C: 49% |
| | | variant, | | | 136,059,904 | | | | TCTTCC | | | G: 39% | G: 11% | G: 49% | G: 43% | G: 52% | G: 51% |
| | | 640bp from | | | | | | | TATGCT | | | | | | | | |
| | | exon 13 | | | | | | | CC | | | | | | | | |
| | | Intronic | | | 136,060,125- | rs6894815 | G/C | ccc | GAGACT | − | 0.42 | C: 58% | C: 76% | C: 50% | C: 57% | C: 48% | C: 49% |
| | | variant, | | | 136,060,225 | | | | GAGACT | | | G: 42% | G: 24% | G: 50% | G: 43% | G: 52% | G: 51% |
| | | 659bp from | | | | | | | GAAGAC | | | | | | | | |
| | | exon 14 | | | | | | | AG | | | | | | | | |
| | | Intronic | | | 136,060,553- | rs10064478 | T/G | cgg | TGCCTG | + | 0.42 | T: 42% | T: 23% | T: 50% | T: 43% | T: 52% | T: 51% |
| | | variant, | | | 136,060,653 | | | | TAATCA | | | G: 58% | G: 77% | G: 50% | G: 57% | G: 48% | G: 49% |
| | | 230bp from | | | | | | | CAGCTA | | | | | | | | |
| | | exon 14 | | | | | | | CT | | | | | | | | |
| Exon | 14 | | 136,060,834 | 136,060,936 | | | | | | | | | | | | | |
| Intron | 14 to 15 | Intronic | 136,060,937 | 136,061,499 | 136,060,930- | rs6880837 | T/C | cca | TCTCTC | − | 0.41 | T: 41% | T: 24% | T: 49% | T: 41% | T: 50% | T: 48% |
| | | variant, | | | 136,061,030 | | | | CACCAA | | | C: 59% | C: 76% | C: 51% | C: 59% | C: 50% | C: 52% |
| | | 44bp from | | | | | | | CTGCCA | | | | | | | | |
| | | exon 14 | | | | | | | CA | | | | | | | | |
| Exon | 15 | | 136,061,500 | 136,061,579 | | | | | | | | | | | | | |
| Intron | 15 to 16 | | 136,061,580 | 136,062,662 | | | | | | | | | | | | | |
| Exon | 16 | | 136,062,663 | 136,062,687 | | | | | | | | | | | | | |
| Intron | 16 to 17 | | 136,062,688 | 136,063,185 | | | | | | | | | | | | | |
| Exon | 17 | | 136,063,186 | 136,063,817 | | | | | | | | | | | | | |

As shown in FIG. 15, the positions of the variants within TGFBI, with most of the SNPs clustered in introns. Thus, multiple TGFBI mutations located in the hotspots in exons 11, 12 and 14 may be targeted simultaneously using this approach. Therefore, a CRISPR Cas 9 system may target more than one patient or one family with a mutation. One CRISPR/Cas9 system designed in this way may be used to treat a range of TGFBI mutations. The CRISPR/Cas9 system may employ an sgRNA adjacent to a PAM site located in the flanking intron that is common to both wild-type and mutant alleles in tandem with a sgRNA adjacent to a PAM site that is specific to the mutant allele (FIG. 16). This would result in NHEJ in the intron of the wild-type allele that should have no functional effect, while in the mutant allele would result in a deletion encompassing the DNA between the two cut sites. This technique is demonstrated in leucocytes isolated from a patient with a suitable SNP profile.

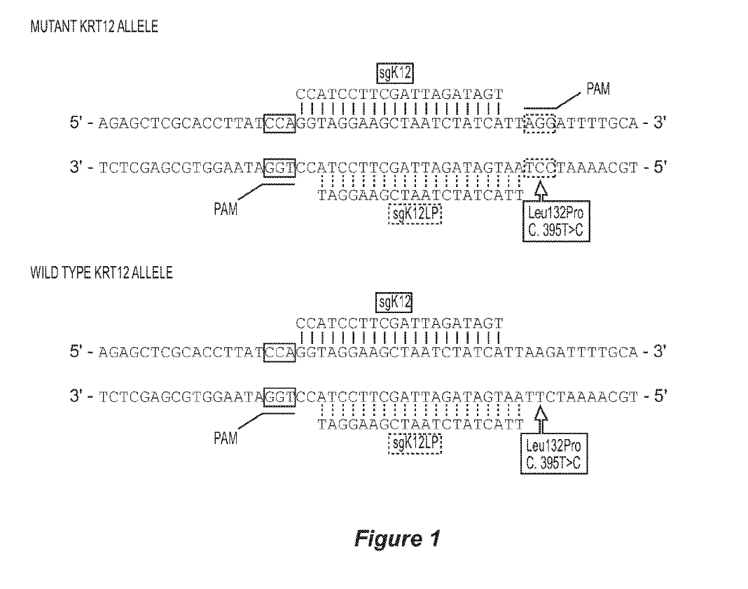

Constructs: Three plasmids expressing Cas9 and an sgRNA were used. The non-targeting plasmid used was pSpCas9(BB)-2A-Puro (PX459) (Broad Institute, MIT; Addgene plasmid 48139; FIG. 7). Following a published protocol (Ran F A, et al., Nat Protoc 2013; 8: 2281-2308), the plasmid containing the sgRNA specific to the K12-L132P allele was designed by annealing and cloning the following 2 primers (Life Technologies, Paisley, UK): 5′-CACCGTAGGAAGCTAATCTATCATT-3′ and 5′-AAACAATGATAGATTAGCTTCCTAC-3′ into pSpCas9(BB)-2A-Puro. This sgRNA corresponds to the 20 nucleotides directly 3′ of the allele-specific PAM found on the K12-L132P allele (FIG. 1, red), hereafter named sgK12LP. A Cas9/sgRNA plasmid to target both wild-type and mutant K12 sequences was constructed (Sigma, Gillingham, UK) and used as a positive control (FIG. 1, green).

Additional K12 expression constructs previously described were used to assess allele specificity and potency. Firefly luciferase plasmids with the full mRNA sequence for either K12-WT or K12-L132P inserted 3′ of the stop codon, hereafter named as K12WT-Luc and KI2LP-Luc, respectively (Liao H, et al. PLoS One 2011; 6: e28582), and expression plasmids for mature haemagglutinin (HA)-tagged K12-WT and K124, l.32P protein (Courtney DvG, et al. Invest Ophthalmol Vis Sci 2014; 55: 3352-3360) with plasmids hereafter known as K12WT-HA and K12LP-HA, respectively, were used. An expression construct for Renilla luciferase (pRL-CMV, Promega, Southampton, UK) was used for the dual-luciferase assay to normalize transfection efficiency.

Dual-luciferase assay: A dual-luciferase assay was used to quantifY potency and allele-specificity of the three test sgRNAs in exogenous constructs, using methods adapted as previously described (Courtney D G, et al. Invest Ophthalmol Vis Sci 2014: 55: 977-985; Allen E H A, et al. Invest Ophthalmol Vis Sci 2013; 54: 494-502; Atkinson S D, et al. J Invest Dermatol 2011; 131: 2079-2086). In short, HEK AD293 cells (Life Technologies) were transfected using Lipofectamine 2000 (Life Technologies) with both K12WT-Luc or K12LP-Luc expression constructs and sgNSC, sgK12 or sgK12LP constructs at a ratio of 1:4. Cells were incubated for 72 h before being lysed and the activities of both Firefly and Renilla luciferase quantified. In all, eight replicates were carried out for each transfection condition.

Western blotting: HA-tagged wild-type (K12WT-HA) and mutant (K12LP-HA) expression constructs (Liao H, et al. PLoS One 2011; 6: e28582.) were transiently co-transfected with each of the sgRNAs at a ratio of 1:4 into HEK AD293 cells in duplicate using Lipofectarnine 2000 (Invitrogen), using similar methods as previously described (Courtney D G, et al. Invest Ophthalmol Vis Sci 2014; 55: 977-985; Allen EHA, et al. Invest Ophthalmol Vis Sci 2013; 54: 494-502). Transfected cells were incubated for 72 h. Expression of HA-tagged K12 and β-actin was analyzed using a rabbit polyclonal antibody to HA (Abeam, Cambridge, UK; ab9110, 1:2000) and a mouse monoclonal antibody to human β-actin (Sigma, 1:15 000) using standard methods (Courtney D G, et al. Invest Ophthalmol Vis Sci 2014; 55: 977-985; Allen E H A, et al. Invest Ophthalmol Vis Sci 2013; 54: 494-502). Membranes were incubated with a secondary horseradish peroxide-conjugated polyclonal swine anti-rabbit antibody (DakoCytomation, Ely, UK) or a horseradish peroxide-conjugated goat anti-mouse antibody (DakoCytomation), respectively. Protein binding was detected by standard chemiluminescence (Life Technologies). Densitometry was performed using Image) (Schneider C A, Rasband W S, Eliceiri K W. Nat Methods 2012; 9: 671-675), to quantify the band intensity of the HA-tagged K12 (n=4). This was normalized to the band intensity of β-actin.

Quantitative real-time PCR: Transfections were carried out in the same manner as described for western blotting; however, both K12WT-HA and K12LP-HA were simultaneously transfected into cells. All transfections were carried out in triplicate. Following transfection, cells were incubated for 48 h and RNA extracted using the RNAeasy Plus kit (Qiagen, Venlo, The Netherlands). Following cDNA conversion of 500 ng of RNA (Life Technologies) quantitative real-time PCR was performed to quantity levels of KRT12 mRNA, A KRT12 assay was used (assay Id 140679; Roche, West Sussex, UK) alongside an HPRT assay (assay ID 102079; Roche) and a GAPDH assay (assay ID 141139; Roche). Each sample was run in triplicate for each assay and relative gene expression was calculated using the ΔΔCT method (Livak K J, Schmittgen T D. Methods 2001; 25: 402-408). KRT12 expression levels were normalized against HPRT and GAPDH, where expression of both reference genes was deemed to be ‘stable’ across treatment groups, using the BestKeeper software tool (Pfafil M W, Tichopad A, Prgornet C, Neuvians T P, Biotechnol Lett 2004; 26: 509-515).

Pyrosequencing: Using the same cDNA samples assessed by quantitative reverse transcriptase-PCR, pyrosequencing was carried out to determine the ratio of remaining K12-L132P mRNA to K12-WT mRNA, exactly as described previously (Courtney D G, et al. Invest Ophthalmol Vis Sci 2014; 55: 3352-3360).

KR172 transgenic mouse: A C57 mouse model was obtained, with a human K12-L132P allele knocked in to replace the endogenous mouse Krt12 coding sequence. This allowed for the in-vivo targeting of KRT12-L132P by the allele-specific sgRNA and Cas9. Female heterozygous mice at 24 weeks old were used, where one copy of the human K12-L132P allele and one copy of murine Krt12 were present. Standard PCR and Sanger dideoxynucleotide sequencing was used to genotype the mice and confirm heterozygosity of the K12-L132P allele. Randomization of animals was not required, as this study investigated the effect of treatment on one cornea, whereas the other cornea of the same animal was used as the negative control. Investigators were not blinded in this study. All experimentation complied with ethical regulations and was approved by the local ethics committee.

In-vivo intrastromal ocular injection: To achieve transient expression of the allele-specific sgRNA and the Cas9, the sgK12LP plasmid was introduced into the corneal stroma of the heterozygous knock-in mice by intrastromal ocular injection, following previously described protocols (Moore J E, McMullen C B T, Mahon G, Adamis A P. DNA Cell Biol 21: 443-451). To assess this delivery method, wild-type mice were first injected with 4 μg of a Cas9-GFP plasmid (pCas9D10A_GFP) (Addgene plasmid 44720). Mice were culled at 24, 48 and 72 h, and corneas fixed in 4% paraformaldehyde and processed using standard histological procedures. Five-micrometer-thick sections were cut, rehydrated and imaged by fluorescent microscopy. Mice were administered with general anesthetic and a local anesthetic to the cornea. A qualified ophthalmologist injected 4 μg of sgK12LP or sgNSC plasmid diluted in a total of 3 μl phosphate-buffered saline into the cornea of the right eye and the left eye, respectively, of four mice. Mice were culled 48 h post treatment.

Sequencing and determination of NHEJ: Once the mice were culled, the eyes were enucleated and the corneas were dissected. gDNA was extracted using a DNA extraction kit (Qiagen) and samples were pooled into two treatment groups: sgK12LP and sgNSC. Samples underwent PCR amplification using the following two primers to amplify the region around the K12-L132P mutation: 5′-ACACCCATCTIGCAGCCTAT-3′ and 5′-AAAATTCCCAAAGCGCCTC-3′. PCR products were gel purified and ligated into the CloneJet cloning vector (Life Technologies) and were used to transform DH5α competent cells (Life Technologies). A total of 13 clones were selected and plasmid DNA prepared using a miniprep kit (Qiagen) following manufacturer's procedures. DNA from the 13 clones was then sequenced (Department of Zoology, University of Oxford) using the sequencing primers provided with the CloneJet vector. The two most likely exonic off-target sites for sgK12LP in the mouse genome, as predicted by the Zhang Lab online tool (crispr.mit.edu) were assessed in the same way, where 10 colonies were selected for analysis for each predicted off target. The predicted off-target sites were 5′-TAAGTAGCTGATCTATCAGIGGG-3′ (Gon4l) and 5′-TGGGAAGCATATCTGTCATTIGG-3′ (Asphd1). Only these two sites were selected, as they were the only two to have a calculated off-target score >0.1.

Statistics: All error bars represent the s.e.m. unless stated otherwise. Significance was calculated using an unpaired t-test, as all samples demonstrated the same distribution. Statistical significance was set at 0.05%. Variance was calculated among groups and deemed to be similar.

Construction of a KRT12-specific sgRNA: An analysis of the sequence changes that result from MECD-causing KRT12 missense mutations revealed that the L132P mutation that causes the severe form of MECD coincidentally results in the generation of a novel PAM site (AAG>AGG). An sgRNA (sgK12LP) complementary to the sequence 20 nucleotides adjacent to the 5′-end of the novel PAM site generated by the KRT12 L132P mutation was designed and assessed for potential off targets using the ‘Optimized CRISPR Design Tool’ provided online by the Zhang lab, MIT 2013, (FIG. 1, red. The sgRNA was calculated as having a score of 66% using this system, where a score >50% is deemed to be of high quality with a limited number of predicted possible off targets.

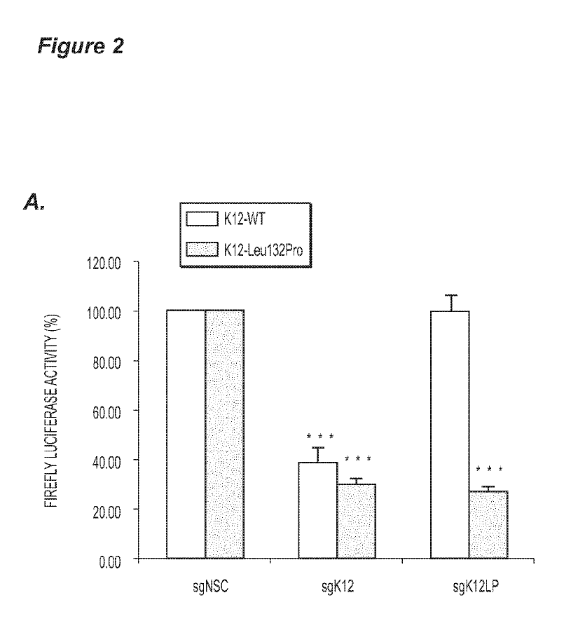

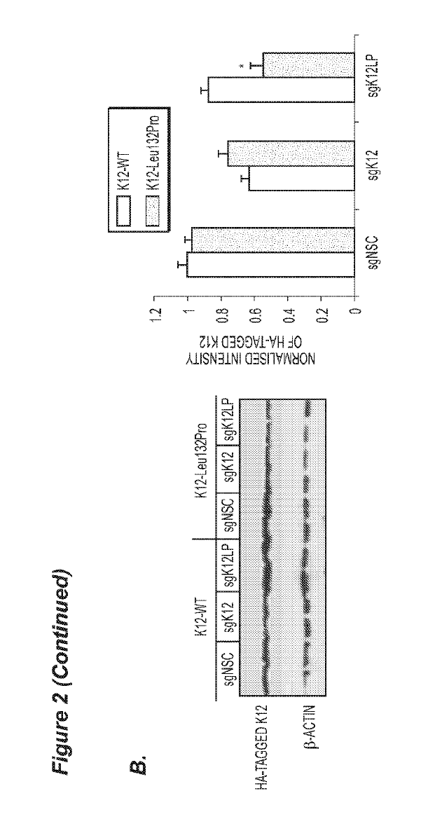

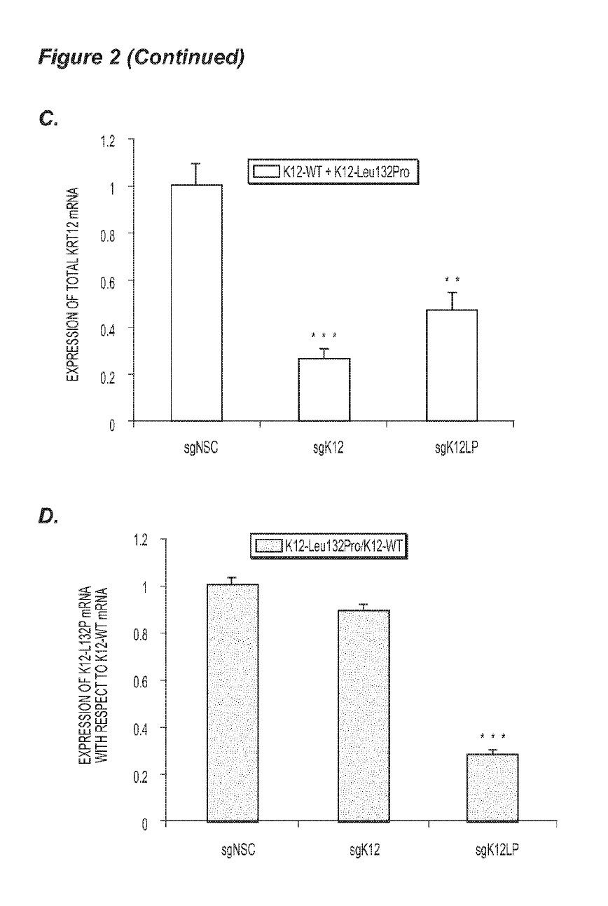

Assessment of sgK12LP allele specificity and potency in vitro: The allele-specificity and potency of sgK12LP was assessed in vitro, in HEK AD293 cells, using exogenous expression constructs for wild-type and mutant K12. Allele specificity was first determined using a dual-luciferase reporter assay (FIG. 2a). Firefly luciferase activity was found to be significantly decreased in cells expressing either K12WT-Luc or K12LP-Luc and treated with sgK12. A potent and allele-specific reduction of firefly luciferase activity was observed in cells treated with sgK12LP. In cells expressing K12LP-Luc, a reduction of 73.4±2.7% (P<0.001) was observed (FIG. 2a). This allele-specific and potent knockdown was also observed by western blotting, in cells expressing either K12WT-HA or K12LP-HA (FIG. 2b; image representative of four blots) and quantification by densitometry revealed a significant reduction of 32% in K12LP-HA protein by sgK12LP in comparison with K12WT-HA protein (P<0.05). In cells treated with sgK12, both wild-type and mutant K12 protein was found to have decreased, whereas in those treated with sgK12LP there appeared to be no effect on expression of the wild-type protein but a significant knockdown of the mutant K12 protein (FIG. 2b).

To support this data, observed at the protein level, quantitative reverse transcriptase-PCR and pyrosequencing were carried out to determine allele specificity and potency at the mRNA level. In cells expressing both wild-type and mutant K12 simultaneously (in a 1:1 expression ratio) and treated with each of the three test Cas9/sgRNA expression constructs (NSC, K12 and K12LP), quantitative reverse transcriptase-PCR was used to determine knockdown of total K12 mRNA (FIG. 2c). A potent reduction of 73.1±4.2% (P<0.001) of total K12 mRNA was observed in sgK12-treated cells, with a lesser reduction of 52.6±7.0% (P<0.01) measured in sgK12LP-treated cells (FIG. 2c). Pyrosequencing was used to determine the intracellular proportion of the remaining mature mRNA species after treatment with these sgRNAs (FIG. 2d). Proportions of mRNA were calculated as ‘percentage of K12-L132P′/percentage of K12-WT’. Cells treated with sgNSC were normalized to 1, assuming a ratio of 1:1 between mutant and wild-type K12 mRNA. In cells tested with sgK12, a K12 mutant mRNA proportion of 0.89±0.03 was observed, but the difference to the NSC control was not significant (P<0.14). In those cells treated with sgK12LP, a K12 mutant mRNA proportion of 0.28±0.02 was detected and was significantly altered in comparison with the sgNSC-treated cells (P<0.001) (FIG. 2d).

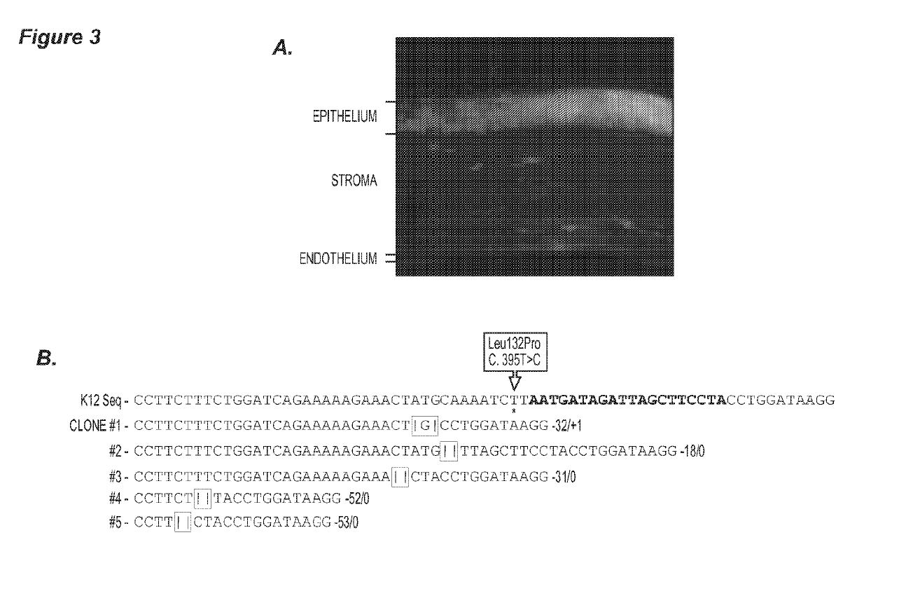

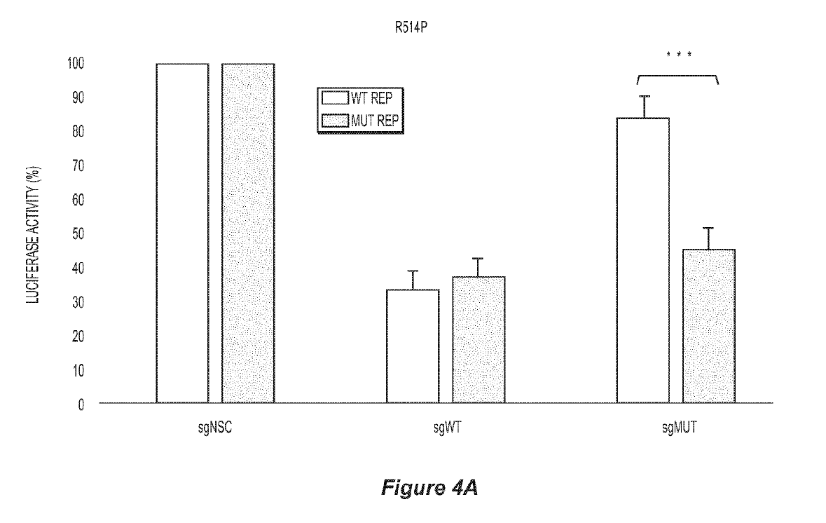

Determination of the efficacy of sgRNA-K12LP in vivo: Intrastromal injection of the Cas9-GFP construct resulted in the presence of green fluorescent protein (GFP) protein in the corneal epithelium at 24 h post injection (FIG. 3a). Transient expression of GFP was found up to 48 h post injection. Following intrastromal injection of either the sgK12LP or sgNSC expression constructs into K12-L132P humanized heterozygous mice and an incubation period of 48 h, mice were euthanized and genomic DNA (gDNA) prepared from the corneas. gDNA from the corneas of four sgK12LP- or sgNSC-treated animals was pooled and PCR amplification of exon 1 of the humanized K12-L132P gene, cloning and sequencing was performed. Of 10 clones established from gDNA of eyes treated with sgNSC, the K12-L132P sequence remained intact in all. Thirteen individual clones from sgK12LP-treated eyes were sequenced; eight were found to contain an unaltered KRT12 L 132P human sequence, whereas five clones demonstrated NHEJ around the predicted cleavage site of the Cas9/sgK12LP complex (FIG. 3b). In one clone (1), an insertion of 1 nucleotide was found, with a deletion of 32 nucleotides. Large deletions of up to 53 nucleotides were observed in vivo (clone 5). Of these 5 clones, 4 contained deletions (clones 1 and 3-5) that are predicted to result in a frameshift that would lead to the occurrence of an early stop codon. The top 2 predicted exonic off-target sites of sgK12LP in mouse were also assessed using this method. Ten clones were sequenced for each target and none were found to have undergone nonspecific cleavage.

TFBI Mutations associated with a PAM site created by mutation in R514P, L518R, L509R and L527R: Single guide RNAs were designed to target each of these mutations and cloned into the sgRNA/Cas9 expression plasmid. In addition, a positive control guide RNA utilizing a naturally-occurring near-by PAM was designed for each mutation. Wild-type and mutant target sequences were cloned into a luciferase reporter plasmid to allow us to monitor the effect of gene editing on expression of WT and MUT expression. Both plasmids were used to transfect AD293 cells and luciferase expression was measured 72 hrs after CRISPR Cas9 treatment using our high throughput reporter gene assay to give a measurement of the amount of MUT and WT DNA present in the cells.

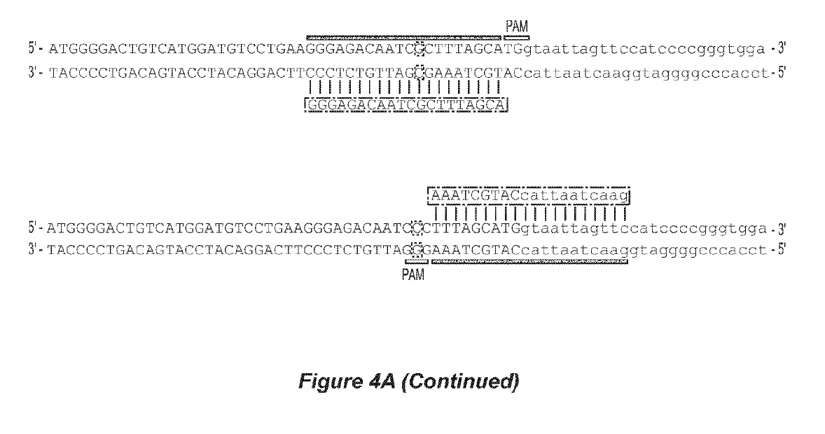

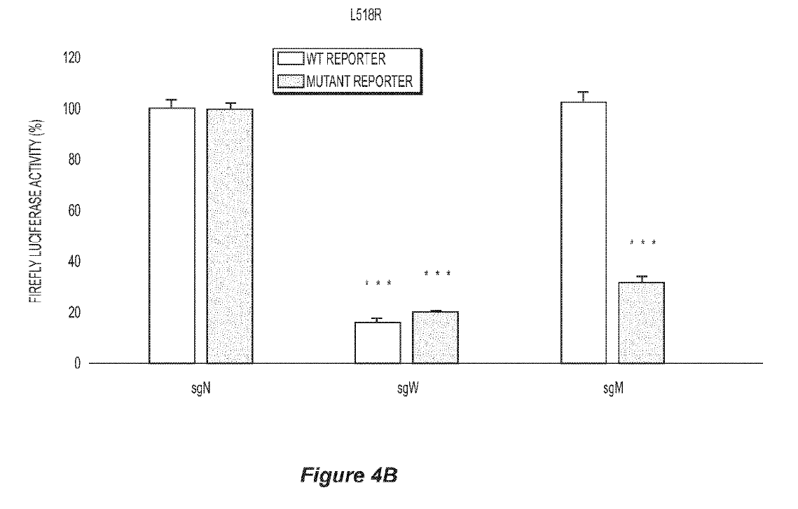

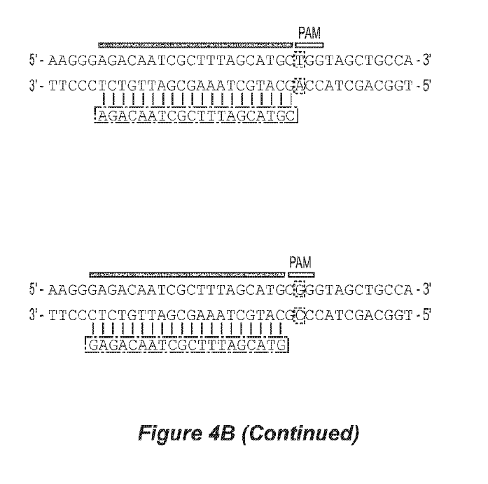

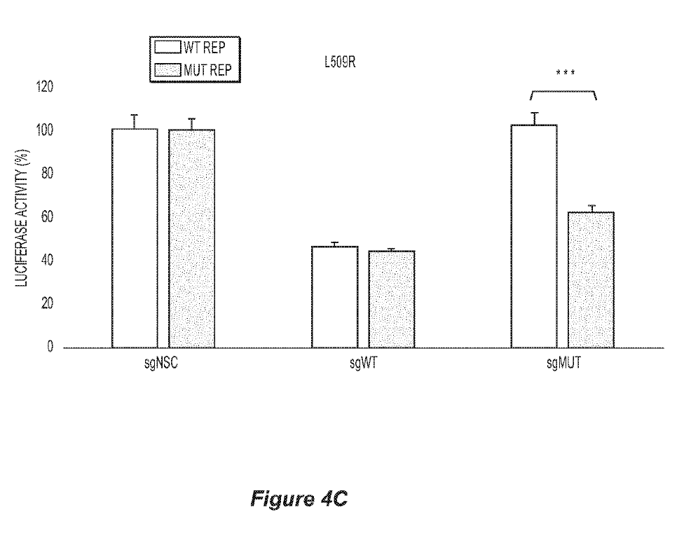

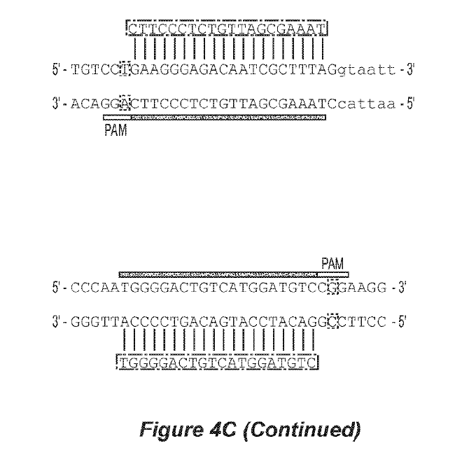

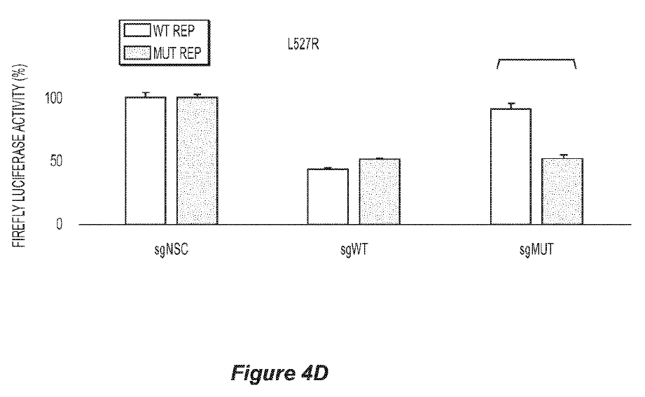

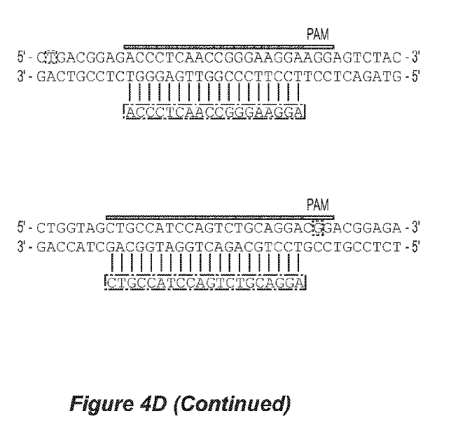

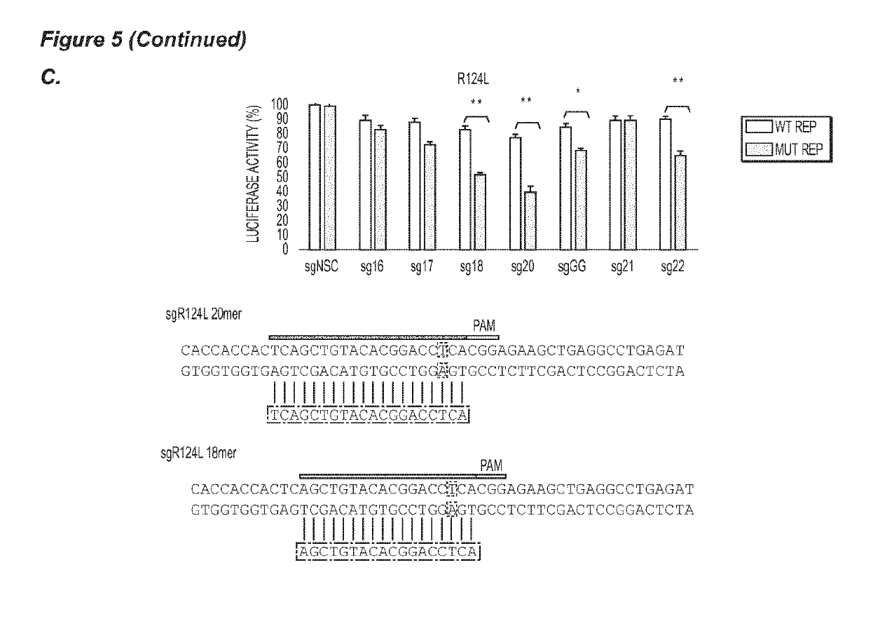

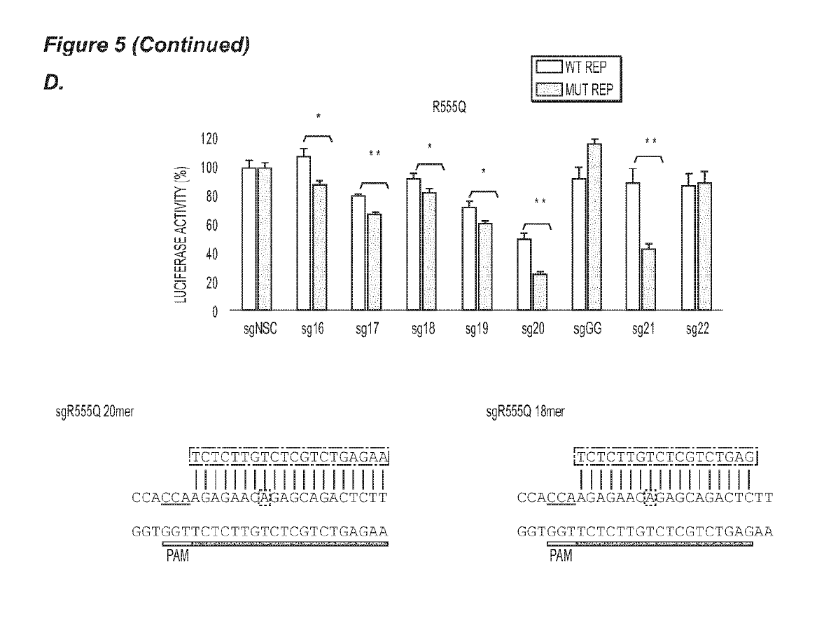

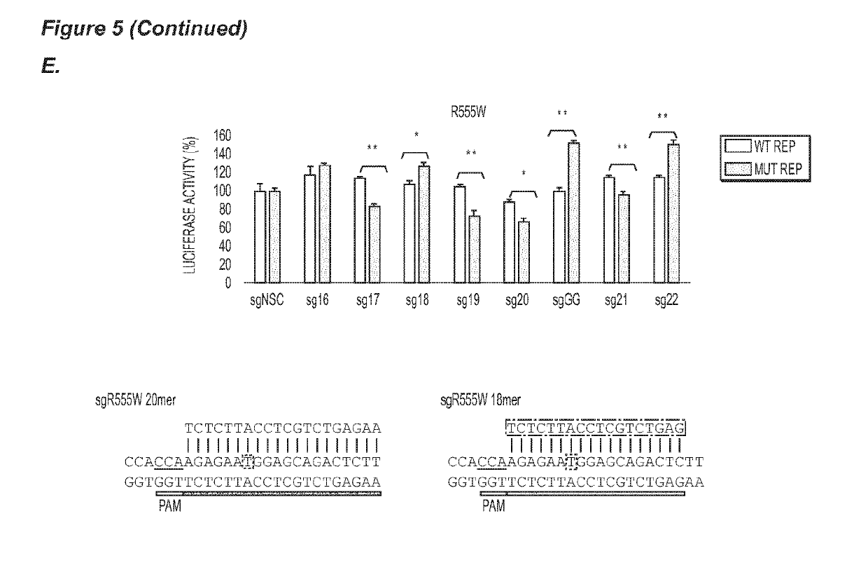

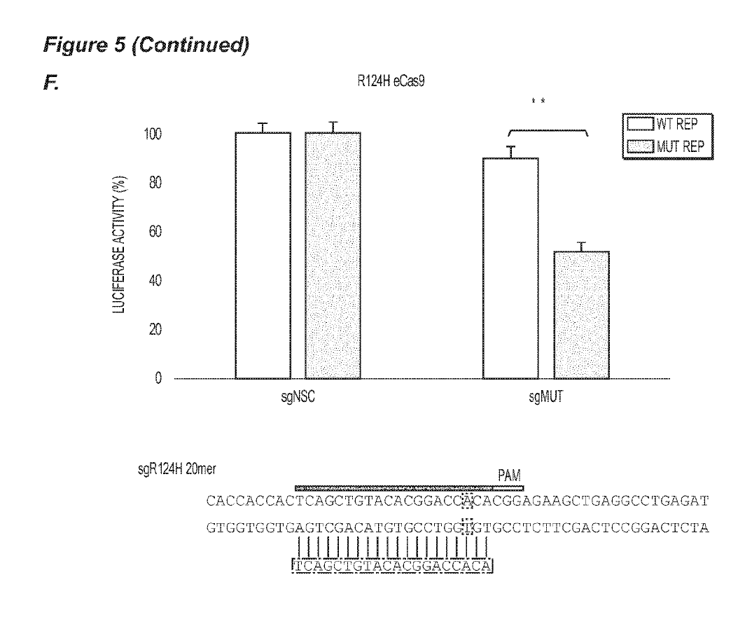

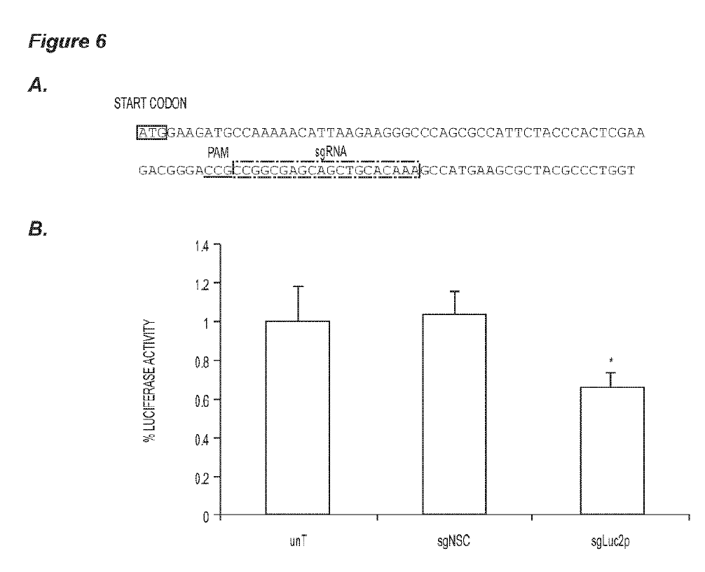

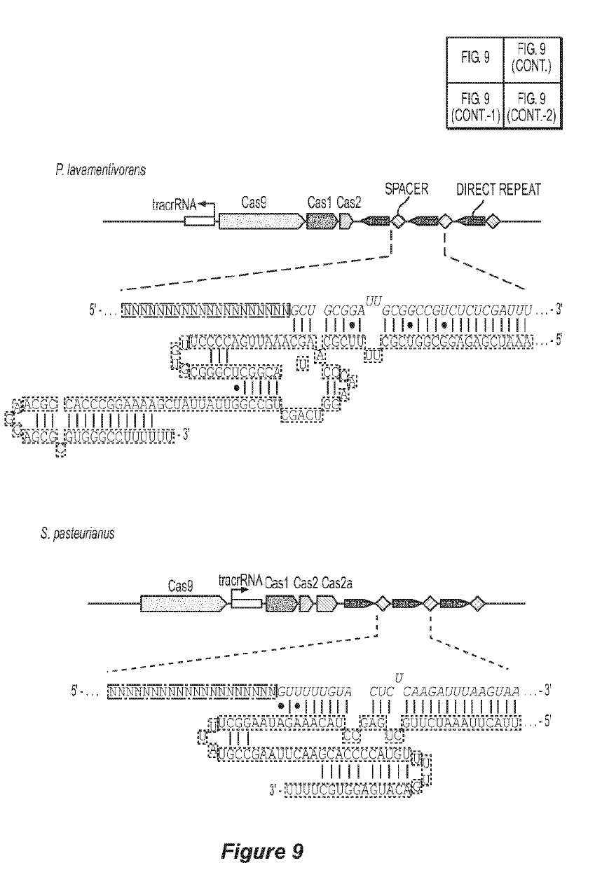

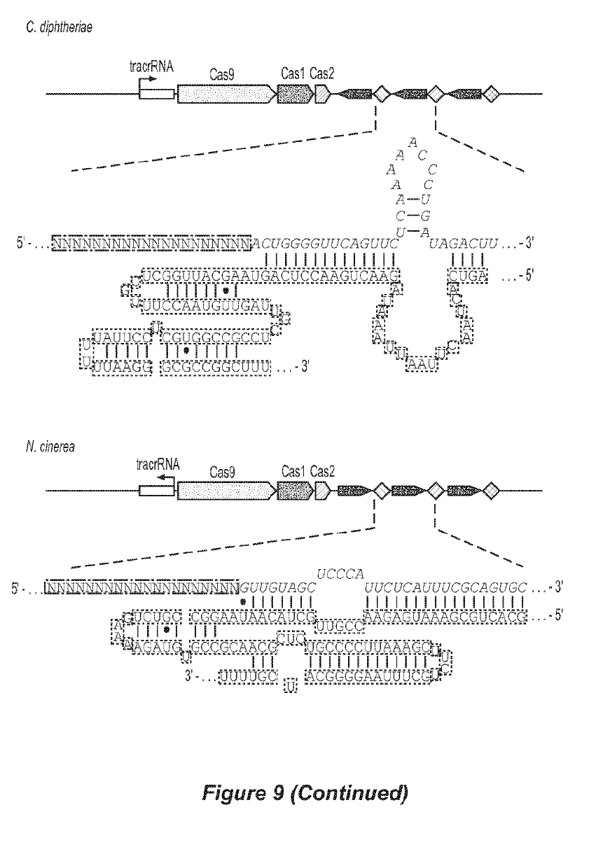

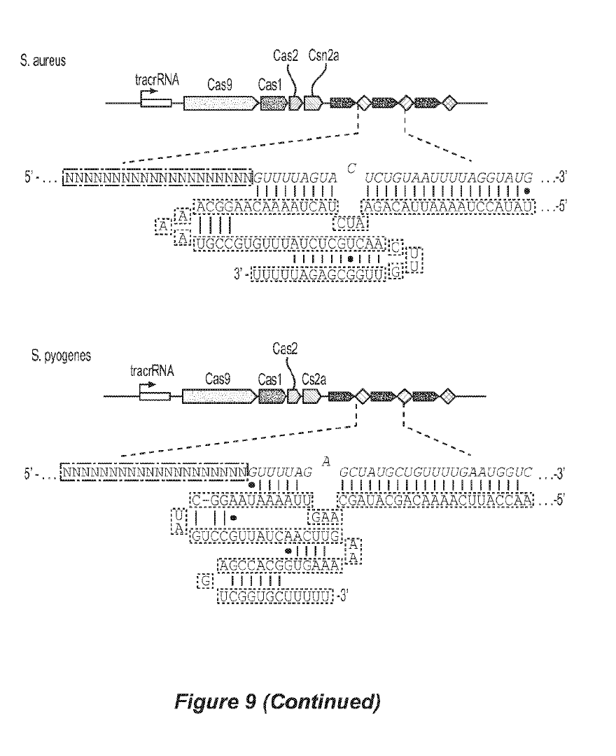

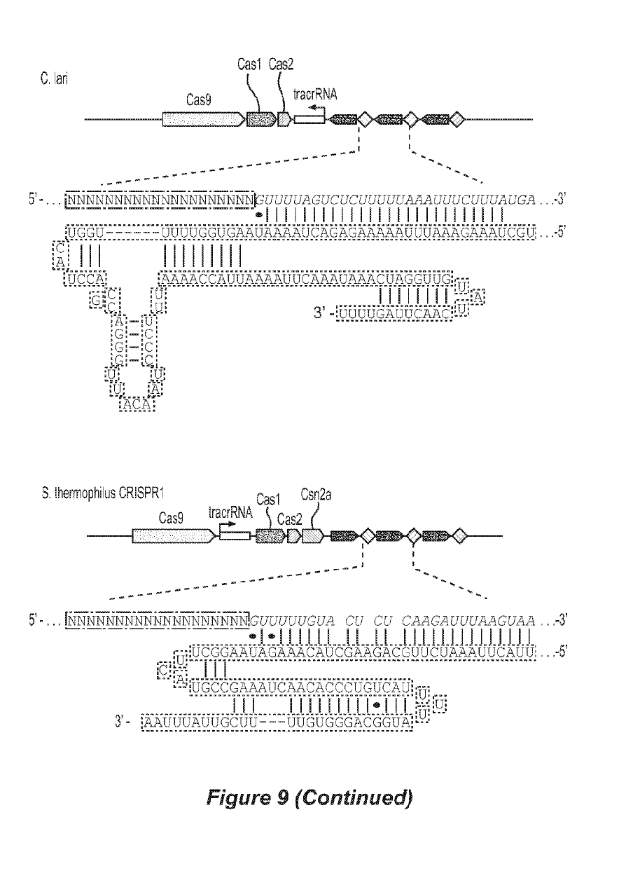

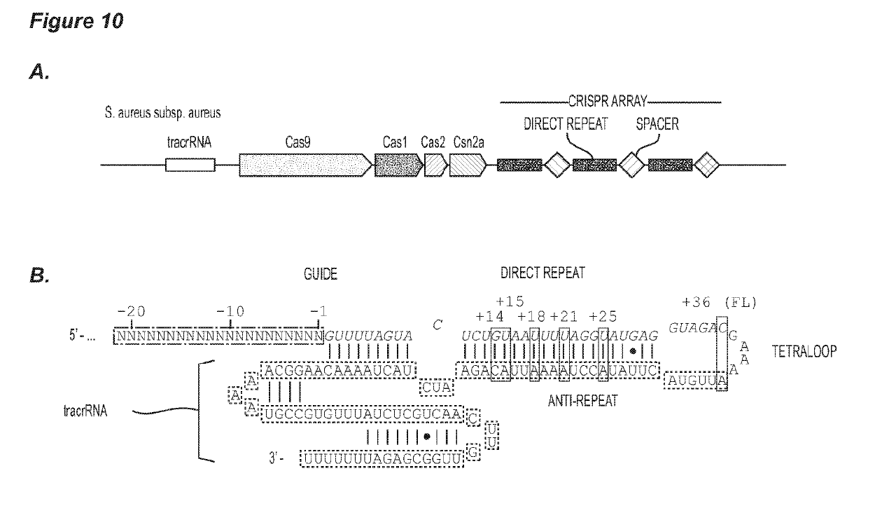

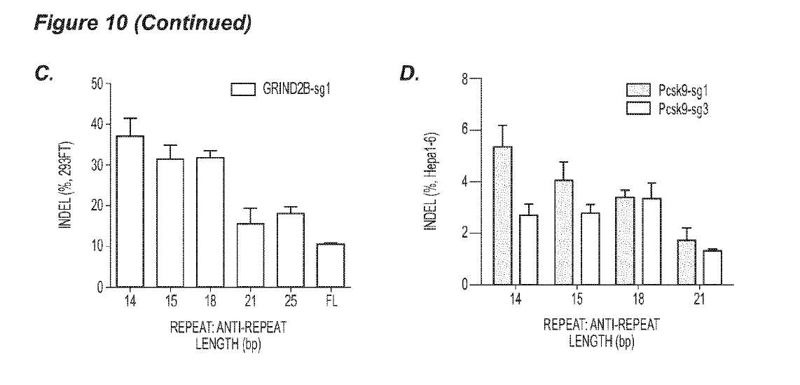

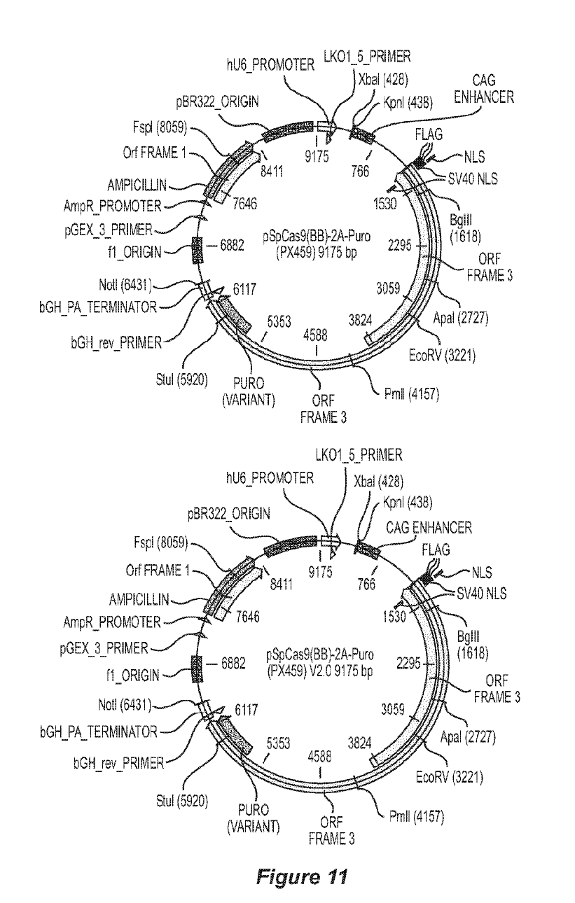

FIG. 4 below shows that for each of these 2 TGFBI mutations (R514P, L518R, L509R and L527R) assessed using the SNP derived PAM approach significant allele-specificity was achieved, with the mutant allele cut by the CRISPR Cas9 system and the WI DNA cut to some degree for some of the guides.

TGFBI Mutations associated with a SNP mutation that lies within a target region adjacent to a PAM site: Single guide RNAs were designed to target these mutations and cloned into the sgRNA/Cas9 expression plasmid. Wild-type and mutant target sequences were cloned into a luciferase reporter plasmid and assessed in our high throughout reporter gene assay. Both plasmids were used to transfect AD293 cells and luciferase expression measured three days afterwards.

Guides ranging in lengths from 16 mer to 22 mer were assessed to determine which length achieves maximal allele-specificity to improve specificity. In addition to the guide length, whether the addition of a double guanine to the 5′ end of the guide would help improve specificity was also assessed. The guide sequences showed different cutting efficiencies based on the guide lengths, and the addition of a double guanine generally did not improve cutting efficiencies (FIG. 5, items A-E).

To improve allele-specificity, a 20 mer guide targeting R124H was cloned into an enhanced Cas9 plasmid. The enhanced Cas9 has been rationally engineered to prevent non-target cutting. A notable reduction in wild type sequence cleavage and an increase in allele specificity (e.g., a difference between the cutting efficiency for a wild type sequence and the cutting efficiency for a mutant sequence) were observed via a dual luciferase assay (FIG. 5, item F).

To confirm DNA cleavage, double-stranded DNA templates were prepared containing either wild type TGFBI sequence or mutant TGFBI sequence. Templates were incubated with synthetic guides and Cas9 protein in vitro at 37° C. for 1 hour. Fragment analysis was conducted on an agaros,-, gel to determine cutting abilities (FIG. 5, item (3).

Additional In Vivo Studies

Live animal imaging: All mice used for live imaging were aged between 12 and 25 weeks old. For imaging, mice were anesthetized using 1.5-2% isoflurane (Abbott Laboratories Ltd., Berkshire, UK) in ˜1.5 I/min flow of oxygen. A mix of luciferin substrate (30 mg/ml D-luciferin potassium salt; Gold Biotechnology, St. Louis, USA) mixed 1:1 w/v with Viscotears gel (Novartis, Camberley, UK) was dropped onto the eye of heterozygous Krt12+/luc2 transgenic mice immediately prior to imaging. A Xenogen IVIS Lumina (Perkin Elmer, Cambridge, UK) was used to quantify luminescence. A region of interest encircling the mouse eye was selected for quantification whose size and shape was kept constant throughout, using protocols as previously described. Fluorescence was also visualized using a Xenogen IVIS Lumina in mice injected with a Cy3-labelled siRNA.

Intrastromal infection: Cas9/sgRNA constructs were delivered to the mouse cornea by intrastromal injection. This was performed by a trained ophthalmic surgeon (J.E.M.), as previously described. To assess the distribution of nucleic acids within the cornea, 2 μl of 150 pmol/μl Cy3-labeled Accell-modified siRNA were injected intrastromally in to the right eyes of WT C57BL/6J mice. To assess the persistence of Cy3-labelled siRNA, animals underwent live imaging on the Xenogen IVIS Lumina system at 0, 6, 24, 48 and 72 hours post-injection (n=3). In addition, mice were sacrificed at 0, 6 and 12 hours after injection (n=3), ocular tissue was removed and frozen at −80° C. Tissue was fixed in OCT and cryosectioned for fluorescence microscopy.

Generation of Cas9/sgRNA expression constructs: A plasmid expressing both Cas9 and an sgRNA, pSpCas9(BB)-2A-Puro (PX459), was obtained as a gift from Professor Feng Zhang (Broad Institute, MIT; Addgene plasmid #48139). An sgRNA targeting luc2 was designed within 61 bp of the start codon, with the aid of the Zhang Lab CRISPR design tool www.crispr.mit.edu). The luc2-specific sgRNA was constructed by first annealing the oligonucleotides 5′ CAC CGT TTG TGC AGC TGC TCG CCG G 3′ and 5′ AAA CCC GGC GAG CAG CTG CAC AAA C 3′, followed by ligation into BbsI-digested pSpCas9(BB)-2A-Puro (PX459); this plasmid as designated as sgLuc2. The original pSpCas9(BB)-2A-Puro plasmid was used as a non-targeting negative control, designated as sgNSC. Activity of the sgLuc2 plasmid was assessed using a similar dual luciferase method to one previously described to evaluate Cas9/sgRNA efficacy. Briefly, a luc2 construct (pGL4.17, Promega) was co-transfected with either sgLuc2 or sgNSC, both Cas9/sgRNA expression constructs, at a molar ratio of 1:4, with a Renilla luciferase expression construct. Cells were incubated for 48 hours post-transgection before luciferase quantification, as described previously.

In vivo assessment of CRISPR/Cas9: The potency of the Cas9/sgLuc2 plasmid was assessed in vivo in the K12-luc2 transgenic mouse using a modified protocol to that used for the assessment of siRNA gene silencing. Both sgLuc2 (right eye) and sgNSC (left eye) were injected intrastromally in a total volume of 4 μl of PBS at a concentration of 500 ng/μl. Live images of mice (n=4) were taken every 24 hours for 7 days, then once every week thereafter for six weeks (42 days) in total. Quantification of luciferase inhibition was determined by calculating the right/left ratio, with values normalized to those at day 0 (as 100%).

In this experiment transgenic mouse models which exclusively expressed Luc2 in the corneal epithelial cells, a CRISPR Cas9 guide was made to target the Luc2 gene as shown below (sgRNA) by way of being able to visually show successful gene editing in the corneal epithelium by viewing Luc2 expression. So in essence this mimics Krt12 expression as it is likewise expressed exclusively in the corneal epithelium. This in vitro dual-luciferase assay demonstrated successful targeting of Luc2 by the sgLuc2P construct, as shown by a significant reduction (* shown to represent p<0.05) in luciferase activity when normalized to untreated cells (data normalized against the untreated control=100%) (FIG. 6). The CRISPR Cas9 sgLuc2 guide was tested in our transgenic mice expressing Luc2 in the cornea. Transgenic mice were made to mimic K12 expression so where there is bright green there is a lot of Krt12 expression, in FIG. 7, blue indicates less Krt12 expression and black means no Krt12 expression at all. The eye on the right was injected with the test sgLuc2 and the eye on the left was injected with the non-targeting non-specific control guide and CRISPR.

As shown in the graph of FIG. 7, the amount of Luc2 expression was measured. After treatment, the corneal luciferase activity of each mouse was quantified using a Xenogen IVIS live animal imager every day for 7 days , then ever 7 days thereafter , for a total of 6 weeks. Luciferase activity for each treatment group expressed as a percentage of control (R/L ratio %).

Confirming Allele-Specific Indels

EBV transformation of ivmphocytes: A sample of 5 ml of whole blood was taken and place in a sterile 50 ml Falcon tube. An equal volume of RPM media containing 20% foetal calf serum was added to the whole blood-mix by gently inverting the tube. 6.26 ml of Ficoll-Paque PLUS (GE Healthcare cat no. 17-1440-02) was placed in a separate sterile 50 ml Falcon tube. 10 ml of blood/media mix was added to the Ficoll-Paque. The tube was spun at 2000 rpm for 20 min at room temperature. The red blood cells formed at the bottom of the tube above which was the Ficoll layer. The lymphocytes formed a layer on top of the Ficoll layer, while the top layer was the medium. A clean sterile Pastette was inserted to draw off the lymphocytes, which were placed in a sterile 15 ml Falcon tube. The lymphocytes were centrifuged and washed. EBV aliquot was thawed and added to resuspended lymphocytes, and the mixture was incubated for 1 hour at 37 degrees C. (infection period). RPMI, 20% FCS media and lmg/int phytohaemagglutinin were added to EBV treated lymphocytes, and the lymphocytes were placed on a 24-well plate.

Electroporation of EBV Transformed Lymphocytes (LCLs): CRISPR constructs (with either CFP or inCherry co-expressed) were added to suspended EBV transfbrmed lymphocytes cells, and the mixture was transferred to an electroporation cuvette. Electroporation was performed, and 500 μl pre-warmed RPMI 1640 media containing 10% FBS was added to the cuvette. The contents of the cuvette was transferred to a 12 well plate containing the remainder of the pre-warmed media, and 6 hours post nucleofection, 1 ml of media was removed and was replaced with fresh media.

Cell sorting of GFP+ and/or mCherry+ Live cells: 24 hours post nucleofection, 1 ml of media was removed and the remaining media containing cells was collected in a 1.5 ml Eppendorf. The cells were centrifuged and resuspended in 200 ul PBS add 50 ul eFlouro 780 viability stain at 1:1000 dilution. After another centrifuge, the cells were resuspended in filter sterile FACS buffer containing 1×HBSS (Ca/Mg++ free), 5 mM EDTA, 25 mM HEPES pH 7.0, 5% FCS/FBS (Heat-Inactivated) and 10 units/mL DNase II. Cells were sorted to isolate live GFP+ and/or mCherry+ cells and were collected in RPMI+20% FBS. Cells were expanded, and DNA was extracted from the cells.

Isolation of single alleles for sequencing: QIAmp DNA Mini Kit (Qiagen) was used to isolate DNA, PCR was used across the region targeted by CRISPR/Cas9. Specific amplification was confirmed by gel electrophoresis, and the PCR product was purified. The PCR product was blunt ended and ligated into pJET1.2/blunt plasmid from the Clonejet Kit (Thermo Scientific). The ligation mixture was transformed into competent DH5α cells. Single colonies were picked, and Sanger Sequencing was performed to confirm edits. The resulting data is shown in FIG. 17.

1. A single guide RNA (sgRNA) designed for CRISPR/Cas9 system for preventing, ameliorating or treating corneal dystrophies.

2. The sgRNA according to claim 1, comprising (i) CRISPR targeting RNA (crRNA) sequence having a nucleotide sequence selected from the group consisting of SEQ ID NO: (10+4n) or SEQ ID NO: (11+4n), in which n is an integer from 0 to 221 and (ii) a trans-activating crRNA (tracrRNA) sequence, wherein the crRNA sequence and tracrRNA sequence do not naturally occur together.

3. The sgRNA according to claim 2, wherein the tracrRNA comprises a nucleotide sequence having at least 85% sequence identity with the nucleotide sequence of SEQ ID NO: 2 or 6.

4. An sgRNA pair designed for CRISPR/Cas9 system, the sgRNA pair comprising
(i) a first sgRNA comprising (a) a first crRNA sequence for a first protospacer adjacent motif (PAM) generating mutation or single-nucleotide polymorphism (SNP) at 3′-end side of a disease-causing mutation or SNP in cis, and (b) a tracrRNA sequence, in which the first crRNA sequence and the tracrRNA sequence do not naturally occur together;
(ii) a second sgRNA comprising (a) a second crRNA guide sequence for a second PAM generating mutation or SNP at 5′-end side of the disease-causing mutation or SNP in cis; (b) a tracrRNA sequence, in which the second crRNA sequence and the tracrRNA sequence do not naturally occur together.

5. The sgRNA pair according to claim 4, wherein the CRISPR/Cas9 system is for preventing, ameliorating or treating corneal dystrophies.

6. The sgRNA pair according to claim 4 or 5, wherein the PAM generating mutations or SNPs are in TGFBI gene.

7. The sgRNA pair according to any one of claims 4-6, wherein the PAM generating mutations or SNPs are in introns of TGFBI gene.

8. The sgRNA pair according to any one of claims 4-7, wherein at least one of the first and second crRNA sequences comprises a nucleotide sequence selected from the group consisting of sequences listed in FIGS. 19-35; and/or at least one of the first and second crRNA sequences comprises a nucleotide sequence selected from the group consisting of sequences listed in Table 2.

9. An engineered Clustered Regularly Interspaced Short Palindromic Repeats (CRISPR)/CRISPR associate protein 9 (Cas9) system comprising (i) at least one vector comprising a nucleotide molecule encoding Cas9 nuclease and the sgRNA of any of claims 1-3 or (ii) at least one vector comprising a nucleotide molecule encoding Cas9 nuclease and the sgRNA pair of any one of claims 4-8, wherein the Cas9 nuclease and said sgRNA pair in the vector do not naturally occur together.

10. The engineered CRISPR/Cas9 system according to claim 9, wherein the Cas9 nuclease is from Streptococcus, Staphylococcus, or variants thereof.

11. The engineered CRISPR/Cas9 system according to any one of claims 9-10, wherein the Cas9 nuclease comprises an amino acid sequence having at least 85% sequence identity with an amino acid sequence selected from the group consisting of SEQ ID NO: 4 or 8.

12. The engineered CRISPR/Cas9 system according to any one of claims 9-11, wherein the nucleotide molecule encoding Cas9 nuclease comprises a nucleotide sequence having at least 85% sequence identity with the nucleotide sequence selected from the group consisting of SEQ ID NO: 3 or 7.

13. The engineered CRISPR/Cas9 system according to any one of claims 9-12, further comprising a repair nucleotide molecule.

14. The engineered CRISPR/Cas9 system according to any one of claims 9-13, further comprising one or more nuclear localization signals (NLSs).

15. The engineered CRISPR/Cas9 system according to any one of claims 9-14, wherein the sgRNA and the Cas9 nuclease are included on the same vector.

16. A method of altering expression of a gene product comprising
introducing the engineered CRISPR″Cas9 system of any one of claims 9-15 into a cell containing and expressing a DNA molecule having a target sequence and encoding the gene product.

17. The method according to claim 16, wherein the engineered CRISPR/Cas9 system comprises
(a) a first regulatory element operably linked to the sgRNA that hybridizes with the target sequence, and
(b) a second regulatory element operably linked to the nucleotide molecule encoding Cas9 nuclease,
wherein the sgRNA targets the target sequence, and the Cas9 nuclease cleaves the DNA molecule.

18. The method according to claim 16 or 17, wherein the cell is a eukaryotic cell.

19. The method according to claim 16 or 17, wherein the cell is a mammalian or human cell.

20. A method of preventing, ameliorating, or treating a disease associated with a mutation or single-nucleotide polymorphism (SNP) in a subject comprising altering expression of the gene product of the subject according to any one of claims 16-19, wherein the DNA molecule comprises a mutant sequence.

21. A method of preventing, ameliorating, or treating corneal dystrophy associated with a gene mutation or single-nucleotide polymorphism (SNP) in a subject, comprising
administering to the subject an engineered CRISPR/Cas9 system comprising at least one vector comprising
(i) a nucleotide molecule encoding Cas9 nuclease, and
(ii) a CRISPR targeting RNA (crRNA) sequence that hybridizes to a nucleotide sequence complementry to a target sequence, the target sequence being adjacent to a 5′-end of a protospacer adjacent motif (PAM), wherein the target sequence or the PAM comprises a mutation or SNP causing the corneal dystrophy,
wherein the nucleotide molecule encoding Cas9 nuclease and the crRNA sequence do not naturally occur together.

22. The method according to claim 21, wherein the PAM comprises the mutation or SNP.

23. The method according to any one of claim 21-22, the crRNA sequence comprises the target sequence, and the crRNA sequence is from 17 to 24 nucleotide long.

24. The method according to any one of claims 21-23, wherein the crRNA sequence consists of the nucleotide sequence selected from the group consisting of SEQ ID NO: (10+4n), in which n is an integer from 0 to 221.

25. The method according to any one of claims 21-24, wherein the PAM and Cas9 nuclease are from Streptococcus or Staphylococcus.

26. The method according to any one of claims 21-25, wherein the PAM consists of NGG or NNGRRT, wherein N is any of A, T, G, and C, and R is A or G.

27. The method according to any one of claims 21-26, wherein the administering comprises introducing the engineered CRISPR/Cas9 system into a cornea of the subject.

28. The method according to any one of claims 21-27, wherein the administering comprises injecting the engineered CRISPR/Cas9 system into a cornea of the subject.

29. The method according to any one of claims 21-28, wherein the administering comprises introducing the engineered CRISPR/Cas9 system into a cell containing and expressing a DNA molecule having the target sequence.

30. The method according to any one of claims 21-29, wherein the corneal dystrophy is selected from the group consisting of Epithelial basement membrane dystrophy (EBMD), Meesmatm corneal dystrophy (MCD), Thiel-Behnke corneal dystrophy (TBCD), Lattice corneal dystrophy (LCD), Granular corneal dystrophy (GCD), and Schnyder conical dystrophy (SCD).

31. The method according to any one of claims 21-30, wherein the SNP is located in a gene selected. from the group consisting of TGFBI, KRT3, KRT12, GSN, and UBIAD1.

32. The method according to any one of claims 21-31, wherein a mutant sequence comprising the gene mutation or SNP encodes a mutant protein selected from the group consisting of
(i) mutant TGFBI proteins comprising Leu509Arg, Arg666Ser Gly623Asp, Arg555Gln, Arg124Cys, Val505Asp, Ile522Asn, Leu569Arg, His572Arg, Arg496Trp, Pro501Thr, Arg514Pro, Phe515Leu, Leu518Pro, Leu518Arg, Leu527Arg, Thr538Pro, Thr538Arg, Val539Asp, Phe540de1, Phe540Ser, Asn544Ser, Ala546Th, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Val624_Val625del, Val624Met, Val625Asp, His626Arg, His626Pro, Val627SerfsX44, Thr629_Asn630AsnValPro, Val631Asp, Arg666Ser, Arg555Trp, Arg124Ser, Asp123delins, Arg124His, Arg124Leu, Leu509Pro, Leu103_Ser104del, Val113Ile, Asp123His, Arg124Leu, and/or Thr125_Glu126del;
(ii) mutant KRT3 proteins with Glu498Val, Arg503Pro, andlor Glu509Lys;
(iii) mutant KRT12 proteins with Met129Thr, Met129Val, Gln130Pro, Leu132Pro, Leu132Va, Leu132His, Asn133Lys, Arg135Gly, Arg135Ile, Ara135Thr, Arg135Ser, Ala137Pro, Leu140Arg, Val143Leu, Val143Leu, Lle391_Leu399dup, Ile 426Val, Ile 426Ser, Tyr429Asp, Tyr429Cys, Arg430Pro, and/or Leu433Arg;
(iv) mutant GSN proteins with Asp214Tyr; and
(v) mutant UBIAD1 proteins with Ala97Thr, Gly98Ser, Asn102Ser, Asp112Asn, Asp112Gly, Asp118Gly, Arg119Gly, Leu121Val, Leu121Phe, Val122Glu, Val122Gly, Ser171Pro, Tyr174Cys, Thr175Ile, Gly177Arg, Lys181Arg, Gly186Arg, Leu188His, Asn232Ser, Asn233His, Asp236Glu, and/or Asp240Asn.

33. The method according to any one of claims 21-32, wherein
(i) a mutant sequence comprising the gene mutation or SNP encodes a mutant TGFBI protein comprising Arg124Cys, and the crRNA sequence comprises SEQ ID NO: 58, 54, 50 or 42;
(ii) a mutant sequence comprising the gene mutation or SNP encodes a mutant TGFBI protein comprising Arg124His, and the crRNA sequence comprises SEQ ID NO: 94, 90, 86, 82, 78, 74 or 70;
(iii) a mutant sequence comprising the gene mutation or SNP encodes a mutant TGFBI protein comprising Arg124Leu, and the crRNA sequence comprises SEQ ID NO: 114, 110, 106 or 98;
(iv) a mutant sequence comprising the gene mutation or SNP encodes a mutant TGFBI protein comprising Arg555Gln; and the crRNA sequence comprises SEQ NO: 178, 174, 170, 166, 162 or 158;
(v) a mutant sequence comprising the gene mutation or SNP encodes a mutant TGFBI protein comprising Arg555Trp, and the crRNA sequence comprises SEQ ID NO: 146, 142, 138, 134, 130 or 126; and/or
(vi) a mutant sequence comprising the gene mutation or SNP encodes a mutant TGFBI protein comprising Leu527Arg, and the crRNA sequence comprises SEQ ID NO: 474, 478, 482 or 486.

34. The method according to any one of claims 21-33, wherein a mutant sequence comprising the gene mutation or SNP encodes a mutant TGFBI protein comprising Arg124His, and the crRNA comprises SEQ ID NO: 86 or 94.

35. The method according to any one of claims 21-34, wherein the corneal dystrophy is associated with the SNP; and the target sequence or the PAM comprises the SNP site causing the corneal distrophy.

36. The method according to any one of claims 21-35, wherein the arget sequence or the PAM comprises a plurality of SNP sites.

37. The method according to any one of claims 21-36, wherein the subject is human.

38. A method of preventing, ameliorating, or treating corneal dystrophy associated with a gene mutation or single-nucleotide polymorphism (SNP) in a subject, comprising
administering to the subject an engineered CRISPR/Cas9 system comprising at least one vector comprising
(i) a nucleotide molecule encoding Cas9 nuclease;
(ii) a first CRISPR targeting RNA (crRNA) sequence that hybridizes to a nucleotide sequence complementary to a first target sequence, the first target sequence being adjacent to the 5′-end of a first protospacer adjacent motif (PAM) at 3′-end side of a disease-causing mutation or SNP in cis, wherein the first target sequence or the first PAM comprises a first ancestral mutation or SNP site,
(iii) a second crRNA sequence that hybridizes to a nucleotide sequence complementary to a second target sequence, the second target sequence being adjacent to the 5′-end of a second PAM at 5′-end side of a disease-causing mutation or SNP in cis, wherein the second target sequence or the second PAM comprises a second ancestral mutation or SNP site,
wherein the at least one vector does not have a nucleotide molecule encoding Cas9 nuclease and a crRNA sequence that naturally occur together.

39. The method according to claim 38, wherein the PAM generating mutations or SNPs are in TGFBI gene.

40. The method according to claim 38 or 39, wherein the PAM generating mutations or SNPs are in introns of TGFBI gene.

41. The method according to any one of claims 38-40, wherein at least one of the first and second crRNA sequences comprises a nucleotide sequence selected from the group consisting of sequences listed in FIGS. 19-35; and/or at least one of the first and second crRNA sequences comprises a nucleotide sequence selected from the group consisting of sequences listed in Table 2.

42. The method according to any one of claims 38-41, wherein the first PAM comprises the first mutation or SNP site and/or the second PAM comprises the second mutation or SNP site.

43. The method according to any one of claims 38-42, wherein
the first crRNA sequence comprises the first target sequence;
the second crRNA sequence comprises the second target sequence;
the first crRNA sequence is from 17 to 24 nucleotide long; and/or
the second crRNA sequence is from 17 to 24 nucleotide long.

44. The method according to any one of claims 38-43, wherein the first and/or second PAMs and the Cas9 nuclease are from Streptococcus or Staphylococcus.

45. The method according to any one of claims 38-44, wherein the first and second PAMs are both from Streptococcus or Staphylococcus.

46. The method according to any one of claims 38-45, wherein the PAM consists of NGG or NNGRRT, wherein N is any of A, T, G, and C, and R is A or G.

47. The method according to any one of claims 38-46, wherein the administering comprises introducing the engineered CRISPR/Cas9 system into a cornea of the subject.

48. The method according to any one of claims 38-47, wherein the administering comprises injecting the engineered CRISPR/Cas9 system into a cornea of the subject.

49. The method according to any one of claims 38-48, wherein the administering comprises introducing the engineered CRISPR/Cas9 system into a cell containing and expressing a DNA molecule having the target sequence.

50. The method according to any one of claims 38-49, wherein the corneal dystrophy is selected from the group consisting of Epithelial basement membrane dystrophy (EBMD), Meesmann corneal dystrophy (MCD), Thiel-Behnke corneal dystrophy (TBCD), Lattice corneal dystrophy (LCD), Granular corneal dystrophy (GCD), and Schnyder conical dystrophy (SCD).

51. The method according to any one of claims 38-50, wherein a mutant sequence copmrises the disease-causing mutation or SNP encodes a mutant protein selected from the group consisting of mutant TGFBI proteins comprising Leu509Arg, Arg666Ser, Gly623Asp, Arg555Gln, Arg124Cys, Val505Asp, Ile522Asn, Leu569Arg, His572Arg, Arg496Trp, Pro501Thr, Arg514Pro, Phe515Leu, Leu518Pro, Leu518Arg, Leu527Arg, Thr538Pro, Thr538Arg, Val539Asp, Phe540del, Phe540Ser, Asn544Ser, Ala546Thr, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Val624_Val625del, Val624Met, Val625Asp, His626Arg, His626Pro, Val627SerfsX44, Thr629_Asn630insAsnValPro, Val631Asp, Arg666Ser, Arg555Trp, Arg124Ser, Asp 123delins, Arg124His, Arg124Leu, Leu509Pro, Leu103_Ser104del, Val113Ile, Asp 123His, Arg124Leu, and/or Thr125_Glu126del.

52. The method according to any one of claims 38-51, wherein
the conical dystrophy associated with the SNP;
the first target sequence or the first PAM comprises the first ancestral SNP site; and/or
the second target sequence or the second PAM comprises the second ancestral SNP site.

53. The method according to any one of claims 38-52, wherein a mutant sequence comprising the disease-causing mutation or SNP encodes a mutant TGFBI protein comprising Arg124His.

54. The method according to any one of claims 38-53, wherein the target sequence or the PAM comprises a plurality of mutation or SNP sites.

55. The method according to any one of claims 38-54, wherein the subject is human.

56. A method of treating corneal dystrophy in a subject in need thereof, comprising:
(a) obtaining a plurality of stem cells comprising a nucleic acid mutation in a conical dystrophy target nucleic acid from the subject;
(b) manipulating the nucleic acid mutation in one or more stem cells of the plurality of stem cells to correct the nucleic acid mutation, thereby forming one or more manipulated stem cells;
(c) isolating the one or more manipulated stem cells; and
(d) transplanting the one or more manipulated stem cells into the subject,
wherein manipulating the nucleic acid mutation in the one or more stem cells of the plurality of stem cells includes performing any of the methods of claims 16-55.
